(19) United States
(12) Patent Application Publication
(10) Pub. No.: US 2007/0185088 A1
(43) Pub. Date: Aug. 9, 2007

(54) M3 muscarinic acetylchoine receptor antagonists

(76) Inventors: Jakob Busch-Petersen, King of Prussia, PA (US); Dramane I. Laine, King of Prussia, PA (US); Michael R. Palovich, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/598,888

(22) PCT Filed: Mar. 17, 2004
(86) PCT No.: PCT/US04/08032

§ 371 Date: Sep. 14, 2006

(51) Int. Cl. A61K 31/55; C07D 417/02; C07D 223/16; C07D 403/02

(52) U.S. Cl. 514/217.01; 540/594

(57) ABSTRACT

Muscarinic Acetylcholine receptor antagonists and methods of using them are provided.

### FIELD OF THE INVENTION

This invention relates to novel thiazole aniline compounds, pharmaceutical compositions, processes for their preparation, and use thereof in treating M₃ muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs, and these receptors can mediate both inhibitory and excitatory actions. For example, in smooth muscle found in the airways, bladder and gastrointestinal tract, M₃ mAChRs mediate contractile responses, (1989. The Muscarinic Receptors. The Humana Press, Inc., Clifton, N.J.).

Muscarinic acetylcholine receptor dysfunction has been noted in a variety of different pathophysiological states. For instance, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation. This mAChR dysfunction results in airway hyperreactivity mediated by increased stimulation of M₃ mAChRs. Similarly, inflammation of the gastrointestinal tract in inflammatory bowel disease (IBD) results in M₃ mAChR-mediated hypermotility (Oprins, J. C. J., H P. Meijer, and J. A. Groot. 2000. Tumor Necrosis Factor-{alpha} Potentiates Ion Secretion Induced by Muscarinic Receptor Activation in the Human Intestinal Epithelial Cell Line HT29cl.19A. Ann NY Acad Sci 915:102-106). Incontinence due to bladder hypercontractility has also been demonstrated to be mediated through increased stimulation of M₃ mAChRs. Thus the identification of subtype-selective mAChR antagonists may be useful as therapeutics in these mAChR-mediated diseases.

Despite the large body of evidence supporting the use of anti-muscarinic receptor therapy for treatment of a variety of disease states, relatively few anti-muscarinic compounds are in use in the clinic. Thus, there remains a need for novel compounds that are capable of causing blockade at M₃ mAChRs. Conditions associated with an increase in stimulation of M₃ mAChRs, such as asthma, COPD, IBD and urinary incontinence would benefit by compounds that are inhibitors of mAChR binding.

### SUMMARY OF THE INVENTION

This invention provides for a method of treating a muscarinic acetylcholine receptor (mAChR) mediated disease, wherein acetylcholine binds to an M₃ mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent.

Compounds of Formula (I) useful in the present invention are represented by the structure: 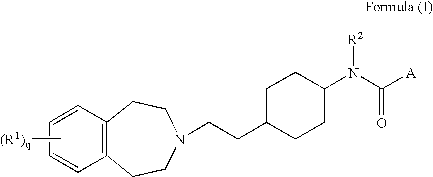
wherein:

R¹ is selected from:the group consisting of a hydrogen or halogen atom; a hydroxy, cyano, nitro, trifluoromethyl, trifluoromethoxy, trifluoromethanesulfonyloxy, pentafluoroethyl, C₁₋₄alkyl, C₁₋₄alkoxy, arylC₁₋₄alkoxy, C₁₋₄alkylthio, C₁₋₄alkoxyC₁₋₄alkyl, C₃₋₆cycloalkylC₁₋₄alkoxy, C₁₋₄alkoxycarbonyl, C₁₋₄alkylsulfonyl, C₁₋₄alkylsulfonyloxy, C₁₋₄alkylsulfonylC₁₋₄alkyl, arylsulfonyl, arylsulfonyloxy, arylsulfonylC₁₋₄alkyl, C₁₋₄alkylsulfonamido, C₁₋₄alkylamido, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl or a arylcarboxamidoC₁₋₄alkyl group; a group R³OCO(CH₂)ₚ, R³CON(R⁴)(CH₂)ₚ, R³R⁴NCO(CH₂)ₚ and R³R⁴NSO₂(CH₂)ₚ, where each of R³ and R⁴ is, independently, selected from a group consisting of a hydrogen atom and a C₁₋₄alkyl group or R³R⁴ forms part of a C₃₋₆azacyloalkane or C₃₋₆(2-oxo)azacycloalkane ring and p represents zero or an integer from 1 to 4; or a group Ar³-Z, wherein Ar³ represents an optionally substituted phenyl ring or an optionally substituted 5- or 6-membered aromatic heterocyclic ring and Z represents a bond, O, S, or CH₂;

R² is selected from the group consisting of a hydrogen atom or a C₁₋₄alkyl group;

q is 1 or 2;

A is selected from the group consisting of a group of the formula (a), (b) (c) and (d): 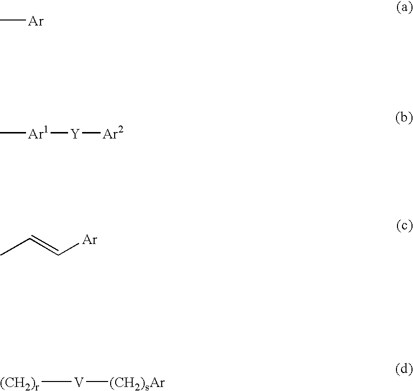
wherein

Ar is selected from a group consisting of an optionally substituted phenyl ring or an optionally substituted 5- or 6-membered aromatic heterocyclic ring; or an optionally substituted bicyclic ring system;

Ar¹ and Ar² are, independently, selected from a group consisting of an optionally substituted phenyl ring or an optionally substituted 5- or 6-membered aromatic heterocyclic ring; and

Y is selected from a group consisting of a bond, —NHCO—, —CONH—, —CH₂—, or —(CH₂)ₘY¹(CH₂)ₙ—, wherein Y¹ represents O, S, SO₂, or CO and m and n each represent zero or 1 such that the sum of m+n is zero or 1; providing that when A represents a group of formula (a), any substituent present in Ar ortho to the carboxamide moiety is necessarily a hydrogen or a methoxy group;

r and s independently represent an integer from zero to 3 such that the sum of r and s is equal to an integer from 1 to 4; and

V represents a bond, O or S;

and salts thereof.

In the compounds of formula (I) above an alkyl group or moiety may be straight or branched. Alkyl groups which may be employed include methyl, ethyl, n-propyl, n-butyl, n-pentyl, n-hexyl and any branched isomers thereof such as isopropyl, t-butyl, sec-butyl, and the like.

When R¹ represents an arylC₁₋₄alkoxy, arylsulfonyl, arylsulfonyloxy, arylsulfonylC₁₋₄alkyl, arylsulfonamido, arylcarboxamido, arylsulfonamidoC₁₋₄alkyl, arylcarboxamidoC₁₋₄alkyl, aroyl, aroylC₁₋₄alkyl, or arylC₁₋₄alkanoyl group, the aryl moiety may be selected from an optionally substituted phenyl ring or an optionally substituted 5 or 6-membered heterocyclic ring. In the group R¹ an aryl moiety may be optionally substituted by one or more substituents selected from hydrogen, halogen, amino, cyano, C₁₋₄alkyl, C₁₋₄alkylamino, C₁₋₄dialkylamino, C₁₋₄alkylamido, C₁₋₄alkanoyl, or R⁵R⁶NCO where each of R⁵ and R⁶ independently represents a hydrogen atom or C₁₋₄alkyl group.

A halogen atom present in the compounds of formula (I) may be fluorine, chlorine, bromine or iodine.

When q is 2, the substituents R¹ may be the same or different.

An optionally substituted 5- or 6-membered heterocyclic aromatic ring, as defined for any of the groups Ar, Ar¹, Ar² or Ar³ may contain from 1 to 4 heteroatoms selected from O, N or S. When the ring contains 2-4 heteroatoms, one is preferably selected from O, N and S and the remaining heteroatoms are preferably N. Examples of 5 and 6-membered heterocyclic groups include furyl, thienyl, pyrrolyl, oxazolyl, thiazolyl, imidazolyl, oxadiazolyl, thiadiazolyl, pyridyl, triazolyl, triazinyl, pyridazyl, pyrimidinyl, pyrazolyl, isothiazolyl, and isoxazolyl.

Examples of bicyclic, for example bicyclic aromatic or heteroaromatic, ring systems for Ar include naphthyl, indazolyl, indolyl, benzofuranyl, benzothienyl, benzothiazolyl, benzimidazolyl, benzoxazolyl, benzisoxazolyl, benzisothiazolyl, quinolinyl, quinoxolinyl, quinazolinyl, cinnolinyl, isoquinolinyl, pyrazolo[1,5-a]pyrimidyl, pyrrolo[3,2-b]pyridyl, pyrrolo[3,2-c]pyridyl, thieno[3,2-b]thiophenyl, 1,2-dihydro-2-oxo-quinolinyl, 3,4-dihydro-3-oxo-2H-benzoxazinyl, 1,2-dihydro-2-oxo-3H-indolyl.

The rings Ar, Ar¹, or Ar² may each independently be optionally substituted by one or more substituents selected from: a hydrogen or halogen atom, or a hydroxy, oxo, cyano, nitro, trifluoromethyl, C₁₋₄alkyl, C₁₋₄alkoxy, C₁₋₄alkylenedioxy, C₁₋₄alkanoyl, C₁₋₄alkylsulfonyl, C₁₋₄alkylsulfinyl, C₁₋₄alkylthio, R⁷SO₂N(R⁸)—, R⁷R⁸NSO₂—, R⁷R⁸N—, R⁷R⁸NCO—, or R⁷CON(R⁸)— group wherein each of R⁷ and R⁸ independently represents a hydrogen atom or a C₁₋₄ alkyl group, or R⁷R⁸ together form a C₃₋₆ alkylene chain.

Alternatively, Ar and Ar² may be optionally substituted by one or more 5- or 6-membered heterocyclic rings, as defined above, optionally substituted by a C₁₋₂ alkyl or R⁷R⁸N— group; wherein R⁷ and R⁸ are as defined above.

In the rings Ar and Ar² substituents positioned ortho to one another may be linked to form a 5- or 6-membered ring.

It will be appreciated that for use in medicine the salts of formula (I) should be physiologically acceptable. Suitable physiologically acceptable salts will be apparent to those skilled in the art and include for example acid addition salts formed with inorganic acids eg. hydrochloric, hydrobromic, sulfuric, nitric or phosphoric acid; and organic acids eg. succinic, maleic, acetic, fumaric, citric, tartaric, benzoic, p-toluenesulfonic, methanesulfonic or naphthalenesulfonic acid. Other non-physiologically acceptable salts eg. oxalates, may be used, for example in the isolation of compounds of formula (I) and are included within the scope of this invention. Also included within the scope of the invention are solvates and hydrates of compounds of formula (I).

Certain of the compounds of formula (I) may form acid addition salts with one or more equivalents of the acid. The present invention includes within its scope all possible stoichiometric and non-stoichiometric forms.

The compounds of formula (I) can exist in the form of cis- and trans-isomers with respect to the configuration at the cyclohexyl ring. When A represents a group (c) the compounds may also exist as geometric isomers around the double bond. The present invention includes within its scope all such isomers, including mixtures. Preferably the compounds of the invention are in the trans configuration with respect to the cyclohexyl ring. For compounds of formula (I) where A represents a group (c), trans geometry of the double bond is preferred.

In compounds of formula (I), it is preferred that R¹ represents a substituent selected from: a halogen atom, methyl, cyano, acetyl, trifluoromethyl, pentafluoroethyl, methylsulphonyl, methylsulphonyloxy or trifluoromethoxy group. Alternatively, it is preferred that R¹ represents a group Ar³Z, where Z is a bond and Ar³ is a 5- or 6-membered ring heterocycle, optionally substituted by a methyl group, containing at least one N and one O atom. Preferably q is 1. R² is preferably a hydrogen atom.

When the group A is a group of formula (a), preferred examples of Ar include optionally substituted phenyl, indolyl, pyrazolo[1,5-a]pyrimidyl, cinnolinyl, quinolinyl, benzo[b]furanyl or pyrrolopyridyl.

When the group A is a group of formula (b), preferred examples of Ar¹ include optionally substituted phenyl, Y is preferably a bond, and preferred examples of Ar² include optionally substituted phenyl, pyridyl, pyrimidinyl, isoxazolyl, oxazolyl or oxadiazolyl.

When the group A is a group of formula (c), preferred examples of Ar include optionally substituted phenyl.

It is also preferred that the rings Ar, Ar¹, or Ar² are each independently optionally substituted by one or more substituents selected from: a hydrogen or halogen atom, cyano, methoxy, trifluoromethyl, methylenedioxy, acetyl, acetylamino, methylsulfonyl, methylsulfonyloxy, methylaminosulfonyl, methylsulfonylamino, or methylaminocarbonyl group.

Certain of the substituted heteroaromatic ring systems included in compounds of formula (I) may exist in one or more tautomeric forms. The present invention includes within its scope all such tautomeric forms, including mixtures.

Particular compounds according to the invention include those specifically exemplified and named hereinafter:
trans-3-(2-(1-(4-(4-Quinolinyl)carboxamido)cyclohexyl)ethyl)-2,3,4,5-tetrahydro-1H-3-benzazepine;
trans-(E)-3-(2-(1-(4-(3-(3-Methylsulfonyl)phenylpropenoyl)amino)cyclohexyl)ethyl)-2,3,4,5-tetrahydro-1H-3-benzazepine;
trans-(E)-3-(2-(1-(4-(3-(4-Fluoro)phenylpropenoyl)amino)cyclohexyl)ethyl)-2,3,4,5-tetrahydro-1H-3-benzazepine;
trans-3-(2-(1-(4-(2-Indolyl)carboxamido)cyclohexyl)ethyl)-2,3,4,5-tetrahydro-1H-3-benzazepine;
trans-3-(2-(1-(4-(3-(3-Pyridyl)phenyl)carboxamido)cyclohexyl)ethyl)-2,3,4,5-tetrahydro-1H-3-benzazepine;
trans-3-(2-(1-(4-Phenylacetamido)cyclohexyl)ethyl)-2,3,4,5-tetrahydro-1H-3-benzazepine;
trans-3-(2-(1-(4-(3-Indolyl)acetamido)cyclohexyl)ethyl)-2,3,4,5-tetrahydro-1H-3-benzazepine;
trans-3-(2-(1-(4-(4-Quinolinyl)acetamido)cyclohexyl)ethyl)-2,3,4,5-tetrahydro-1H-3-benzazepine;
trans-(E)-3-(2-(1-(4-(3-(4-Fluoro)phenylpropenoyl)amino)cyclohexyl)ethyl)-6-methoxy-2,3,4,5-tetrahydro-1H-3-benzazepine;
trans-6-Methoxy-3-(2-(1-(4-(4-quinolinyl)carboxamido)cyclohexyl)ethyl)-2,3,4,5-tetrahydro-1H-3-benzazepine;
trans-6-Methoxy-3-(2-(1-(4-(3-pyrrolo[2,3-b]pyridyl)carboxamido)cyclohexyl)ethyl)-2,3,4,5-tetrahydro-1H-3-benzazepine;
trans-(E)-7-Cyano-3-(2-(1-(4-(3-(4-fluoro)phenylpropenoyl)amino)cyclohexyl)ethyl)-2,3,4,5-tetrahydro-1H-3-benzazepine;
trans-7-Cyano-3-(2-(1-(4-(3-(3-(5-methyl)-1,2,4-oxadiazolyl)benzoyl)amino)cyclohexyl)ethyl)-2,3,4,5-tetrahydro-1H-3-benzazepine;
trans-(E)-7-Cyano-3-(2-(1-(4-(3-(3-acetylamino)phenylpropenoyl)amino)cyclohexyl)ethyl)-2,3,4,5-tetrahydro-1H-3-benzazepine;
trans-7-Cyano-3-(2-(1-(4-(6-(3,4-dihydro-3-oxo)-2H-benzoxazinyl)carboxamido)cyclohexyl)ethyl)-2,3,4,5-tetrahydro-1H-benzazepine;
trans-(E)-7-Cyano-3-(2-(1-(4-(3-(6-(1,2-dihydro-2-oxo)quinolinyl)propenoyl)amino)cyclohexyl)ethyl)-2,3,4,5-tetrahydro-1H-3-benzazepine;
trans-(E)-7-Cyano-3-(2-(1-(4-(3-(2-fluoro-4-acetylamino)phenylpropenoyl)amino)cyclohexyl)ethyl)-2,3,4,5-tetrahydro-1H-3-benzazepine;
trans-(E)-7-Cyano-3-(2-(1-(4-(3-(8-(1,2-dihydro-2-oxo)quinolinyl)propenoyl)amino)cyclohexyl)ethyl)-2,3,4,5-tetrahydro-1H-3-benzazepine;
trans-3-(2-(1-(4-(3-(3-(5-Methyl)-1,2,4-oxadiazolyl)benzoyl)amino)cyclohexyl)ethyl)-7-methylsulphonyl-2,3,4,5-tetrahydro-1H-3-benzazepine;
trans-7-(5-(3-Methyl)-1,2,4-oxadiazolyl)-3-(2-(1-(4-(3-pyrrolo[2,3-b]pyridyl)carboxamido)cyclohexyl)ethyl)-2,3,4,5-tetrahydro-1H-3-benzazepine;
trans-(E)-7-(3-(5-Methyl)-1,2,4-oxadiazolyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(5-(3-Methyl)isoxazolyl)-3-(2-(1-(4-(3-(4-fluoro)phenylpropenoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(4-fluoro)phenylacetamido)cyclohexyl)ethyl)-2,3,4,5-tetrahydro-1H-3-benzazepine;
trans-7-Cyano-3-(2-(1-(4-(2,5-difluoro)phenylacetamido)cyclohexyl)ethyl)-2,3,4,5-tetrahydro-1H-3-benzazepine;
trans-(E)-7-Cyano-3-(2-(1-(4-(3-(2,4-difluoro)phenylpropenoyl)amino)cyclohexyl)ethyl)-2,3,4,5-tetrahydro-1H-3-benzazepine;
trans-(E)-7-Cyano-3-(2-(1-(4-(3-(2,5-difluoro)phenylpropenoyl)amino)cyclohexyl)ethyl)-2,3,4,5-tetrahydro-1H-3-benzazepine;
trans-7-Cyano-3-(2-(1-(4-(3-phenylpropanoyl)amino)cyclohexyl)ethyl)-2,3,4,5-tetrahydro-1H-3-benzazepine;
trans-(E)-7-Cyano-3-(2-(1-(4-(3-(4-methoxy)phenylpropenoyl)amino)cyclohexyl)ethyl)-2,3,4,5-tetrahydro-1H-3-benzazepine;
trans-(E)-7-Cyano-3-(2-(1-(4-(3-(2-acetyl)phenylpropenoyl)amino)cyclohexyl)ethyl)-2,3,4,5-tetrahydro-1H-3-benzazepine;
trans-(E)-7-Cyano-3-(2-(1-(4-(3-(4-acetyl)phenylpropenoyl)amino)cyclohexyl)ethyl)-2,3,4,5-tetrahydro-1H-3-benzazepine;
trans-(E)-7-Cyano-3-(2-(1-(4-(3-(2-cyano)phenylpropenoyl)amino)cyclohexyl)ethyl)-2,3,4,5-tetrahydro-1H-3-benzazepine;
trans-(E)-7-Cyano-3-(2-(1-(4-(3-(3-cyano)phenylpropenoyl)amino)cyclohexyl)ethyl)-2,3,4,5-tetrahydro-1H-3-benzazepine;
trans-7-Cyano-3-(2-(1-(4-(3-(5-(3-methyl)isoxazolyl)benzoyl)amino)cyclohexyl)ethyl)-2,3,4,5-tetrahydro-1H-3-benzazepine;
trans-(Z)-7-Cyano-3-(2-(1-(4-(3-phenylpropenoyl)amino)cyclohexyl)ethyl)-2,3,4,5-tetrahydro-1H-3-benzazepine;
trans-(E)-7-Cyano-3-(2-(1-(4-(3-(2-pyridyl)propenoyl)amino)cyclohexyl)ethyl)-2,3,4,5-tetrahydro-1H-3-benzazepine;
trans-(E)-7-Cyano-3-(2-(1-(4-(3-(1-(4-fluoro)naphthyl)propenoyl)amino)cyclohexyl)ethyl)-2,3,4,5-tetrahydro-1H-3-benzazepine;
trans-(E)-7-Cyano-3-(2-(1-(4-(3-(6-benzodioxanyl)propenoyl)amino)cyclohexyl)ethyl)-2,3,4,5-tetrahydro-1H-3-benzazepine;
trans-(E)-7-Cyano-3-(2-(1-(4-(3-(3-(5-fluoro)indolyl)propenoyl)amino)cyclohexyl)ethyl)-2,3,4,5-tetrahydro-1H-3-benzazepine;
trans-(E)-7-Cyano-3-(2-(1-(4-(3-(3-(1-methyl)benzimidazolyl)propenoyl)amino)cyclohexyl)ethyl)-2,3,4,5-tetrahydro-1H-3-benzazepine;
trans-(E)-7-Cyano-3-(2-(1-(4-(3-(7-benzofuranyl)propenoyl)amino)cyclohexyl)ethyl)-2,3,4,5-tetrahydro-1H-3-benzazepine;
trans-(E)-7-Cyano-3-(2-(1-(4-(3-(5-(3-methyl)indolyl)propenoyl)amino)cyclohexyl)ethyl)-2,3,4,5-tetrahydro-1H-3-benzazepine;
trans-(E)-7-Cyano-3-(2-(1-(4-(3-(6-(2,3-dihydro-2-oxo)indolyl)propenoyl)amino)cyclohexyl)ethyl)-2,3,4,5-tetrahydro-1H-3-benzazepine;
trans-7-Cyano-3-(2-(1-(2-benzofuranylacetamido)cyclohexyl)ethyl)-2,3,4,5-tetrahydro-1H-3-benzazepine;
trans-(E)-7-Cyano-3-(2-(1-(4-(3-(4-(2-methyl)indolyl)propenoyl)amino)cyclohexyl)ethyl)-2,3,4,5-tetrahydro-1H-3-benzazepine;
trans-(E)-7-Cyano-3-(2-(1-(4-(3-(5-benzimidazolyl)propenoyl)amino)cyclohexyl)ethyl)-2,3,4,5-tetrahydro-1H-3-benzazepine;
trans-(E)-7-Cyano-3-(2-(1-(4-(3-phenylpropenoyl)amino)cyclohexyl)ethyl)-2,3,4,5-tetrahydro-1H-3-benzazepine;
trans-(E)-7-Cyano-3-(2-(1-(4-(3-(2,3-methylenedioxy)phenylpropenoyl)amino)cyclohexyl)ethyl)-2,3,4,5-tetrahydro-1H-3-benzazepine;
trans-(E)-7-Cyano-3-(2-(1-(4-(3-(3-(1-(2-oxo)pyrrolidinyl))phenylpropenoyl)amino)cyclohexyl)ethyl)-2,3,4,5-tetrahydro-1H-3-benzazepine;
trans-7-Cyano-3-(2-(1-(4-(2-indolylacetamido)cyclohexyl)ethyl)-2,3,4,5-tetrahydro-1H-3-benzazepine;
trans-7-Cyano-3-(2-(1-(4-(2-benzothiophenylacetamido)cyclohexyl)ethyl)-2,3,4,5-tetrahydro-1H-3-benzazepine;
trans-(E)-7-Cyano-3-(2-(1-(4-(3-(2-(3-bromo)thiophenyl)propenoyl)amino)cyclohexyl)ethyl)-2,3,4,5-tetrahydro-1H-3-benzazepine;
trans-7-Cyano-3-(2-(1-(4-(3-(2-pyridyl)benzoyl)amino)cyclohexyl)ethyl)-2,3,4,5-tetrahydro-1H-3-benzazepine;
trans-7-Cyano-3-(2-(1-(4-(3-(5-pyrimidinyl)benzoyl)amino)cyclohexyl)ethyl)-2,3,4,5-tetrahydro-1H-3-benzazepine;
trans-7-Cyano-3-(2-(1-(4-(3-(4-cyanophenyl)benzoyl)amino)cyclohexyl)ethyl)-2,3,4,5-tetrahydro-1H-3-benzazepine;
trans-(E)-7-Cyano-3-(2-(1-(4-(3-(2-thiophenyl)propenoyl)amino)cyclohexyl)ethyl)-2,3,4,5-tetrahydro-1H-3-benzazepine;
trans-(E)-7-Cyano-3-(2-(1-(4-(3-(2-furanyl)propenoyl)amino)cyclohexyl)ethyl)-2,3,4,5-tetrahydro-1H-3-benzazepine;
trans-(E)-7-Cyano-3-(2-(1-(4-(3-(3-thiophenyl)propenoyl)amino)cyclohexyl)ethyl)-2,3,4,5-tetrahydro-1H-3-benzazepine;
trans-(E)-7-Cyano-3-(2-(1-(4-(3-(3-furanyl)propenoyl)amino)cyclohexyl)ethyl)-2,3,4,5-tetrahydro-1H-3-benzazepine;
trans-(E)-7-Cyano-3-(2-(1-(4-(3-(4-quinolinyl)propenoyl)amino)cyclohexyl)ethyl)-2,3,4,5-tetrahydro-1H-3-benzazepine;
trans-(E)-7-Cyano-3-(2-(1-(4-(3-(5-pyrimidinyl)propenoyl)amino)cyclohexyl)ethyl)-2,3,4,5-tetrahydro-1H-3-benzazepine;
trans-7-Cyano-3-(2-(1-(4-(2,4-difluoro)phenylacetamido)cyclohexyl)ethyl)-2,3,4,5-tetrahydro-1H-3-benzazepine;
trans-7-Cyano-3-(2-(1-(4-(6-(2-amino)benzothiazolyl)acetamido)cyclohexyl)ethyl)-2,3,4,5-tetrahydro-1H-3-benzazepine;
trans-7-Cyano-3-(2-(1-(4-(6-(2-methyl)benzothiazolyl)acetamido)cyclohexyl)ethyl)-2,3,4,5-tetrahydro-1H-3-benzazepine;
trans-(E)-7-Cyano-3-(2-(1-(4-(3-(4-methylaminocarbonyl)phenylpropenoyl)amino)cyclohexyl)ethyl)-2,3,4,5-tetrahydro-1H-3-benzazepine;
trans-7-Cyano-3-(2-(1-(4-(5-(2-amino)benzoxazolyl)acetamido)cyclohexyl)ethyl)-2,3,4,5-tetrahydro-1H-3-benzazepine;
trans-7-Cyano-3-(2-(1-(4-(3-(1-pyrazolyl)benzoyl)amino)cyclohexyl)ethyl)-2,3,4,5-tetrahydro-1H-3-benzazepine;
trans-7-Cyano-3-(2-(1-(4-(2-thiophenyl)acetamido)cyclohexyl)ethyl)-2,3,4,5-tetrahydro-1H-3-benzazepine;
trans-7-Cyano-3-(2-(1-(4-(3-benzothiophenyl)acetamido)cyclohexyl)ethyl)-2,3,4,5-tetrahydro-1H-3-benzazepine;
trans-7-Cyano-3-(2-(1-(4-(3-(2-(5-methyl)-1,3,4-oxadiazolyl)benzoyl)amino)cyclohexyl)ethyl)-2,3,4,5-tetrahydro-1H-3-benzazepine;
trans-(E)-7-Cyano-3-(2-(1-(4-(3-(2-naphthyl)propenoyl)amino)cyclohexyl)ethyl)-2,3,4,5-tetrahydro-1H-3-benzazepine;
trans-(E)-7-Cyano-3-(2-(1-(4-(3-(5-(3-acetyl)indolyl)propenoyl)amino)cyclohexyl)ethyl)-2,3,4,5-tetrahydro-1H-3-benzazepine;
trans-7-Cyano-3-(2-(1-(4-(3-(5-(3-methyl)-1,2,4-oxadiazolyl)benzoyl)amino)cyclohexyl)ethyl)-2,3,4,5-tetrahydro-1H-3-benzazepine;
trans-7-Cyano-3-(2-(1-(4-(5-(2-methyl)benzimidazolyl)acetamido)cyclohexyl)ethyl)-2,3,4,5-tetrahydro-1H-3-benzazepine;
trans-(E)-7-Cyano-3-(2-(1-(4-(3-(3-(2-acetyl)furanyl)propenoyl)amino)cyclohexyl)ethyl)-2,3,4,5-tetrahydro-1H-3-benzazepine;
trans-7-Cyano-3-(2-(1-(4-(6-(2-amino)benzoxazolyl)acetamido)cyclohexyl)ethyl)-2,3,4,5-tetrahydro-1H-3-benzazepine;
trans-7-(5-(3-Methyl)-1,2,4-oxadiazolyl)-3-(2-(1-(4-(2-benzothiophenyl)acetamido)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(3-thienyl)propenoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(5-quinolinyl)carboxamido)cyclohexyl)ethyl)-2,3,4,5-tetrahydro-1H-3-benzazepine;
trans-7-(5-(3-Methyl)-1,2,4-oxadiazolyl)-3-(2-(1-(4-(3-(3-(5-Methyl)-1,2,4-oxadiazolyl)benzoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(8-(1,4-dihydro-4-oxo)quinolinyl)carboxamido)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(3-fluoro)phenylpropenoyl)amino)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(3-acetamido-2-fluoro)phenylpropenoyl)amino)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(3-acetyl)phenylpropenoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(3-fluoro)phenylacetamido)cyclohexyl)ethyl)-2,3,4,5-tetrahydro-1H-3-benzazepine;
trans-7-(5-(3-Methyl)-1,2,4-oxadiazolyl)-3-(2-(1-(4-(2,4-difluoro)phenylacetamido)cyclohexyl)ethyl)-2,3,4,5-tetrahydro-1H-3-benzazepine;
trans-7-(5-(3-Methyl)-1,2,4-oxadiazolyl)-3-(2-(1-(4-(2-naphthyl)acetamido)cyclohexyl)ethyl)-2,3,4,5-tetrahydro-1H-3-benzazepine;
trans-7-(5-(3-Methyl)-1,2,4-oxadiazolyl)-3-(2-(1-(4-(7-(3,4-dihydro-3-oxo)-2H-benzoxazinyl)carboxamido)cyclohexyl)ethyl)-2,3,4,5-tetrahydro-1H-3-benzazepine;
trans-7-(5-(3-Methyl)-1,2,4-oxadiazolyl)-3-(2-(1-(4-(2-fluoro)phenylacetamido)cyclohexyl)ethyl)-2,3,4,5-tetrahydro-1H-3-benzazepine;
trans-3-(2-(1-(4-(5-(2-Methyl)quinolinyl)carboxamide)cyclohexyl)ethyl)-7-methanesulphonyloxy-2,3,4,5-tetrahydro-1H-3-benzazepine;
trans-7-(5-(3-Methyl)-1,2,4-oxadiazolyl)-3-(2-(1-(4-(2-indolyl)carboxamido)cyclohexyl)ethyl)-2,3,4,5-tetrahydro-1H-3-benzazepine;
trans-7-(3-(5-Methyl)-1,2,4-oxadiazolyl)-3-(2-(1-(4-(2-benzothiophenyl)acetamido)cyclohexyl)ethyl)-2,3,4,5-tetrahydro-1H-3-benzazepine;
trans-(E)-7-(3-(5-Methyl)-1,2,4-oxadiazolyl)-3-(2-(1-(4-(3-(3-thienyl)propenoyl)amino)cyclohexyl)ethyl)-2,3,4,5-tetrahydro-1H-3-benzazepine;
trans-7-(3-(5-Methyl)-1,2,4-oxadiazolyl)-3-(2-(1-(4-(5-quinolinyl)carboxamido)cyclohexyl)ethyl)-2,3,4,5-tetrahydro-1H-3-benzazepine;
trans-7-(3-(5-Methyl)-1,2,4-oxadiazolyl)-3-(2-(1-(4-(3-pyrrolo[2,3-b]pyridyl)carboxamido)cyclohexyl)ethyl)-2,3,4,5-tetrahydro-1H-3-benzazepine;
trans-7-(3-(5-Methyl)-1,2,4-oxadiazolyl)-3-(2-(1-(4-(8-(1,4-dihydro-4-oxo)quinolinyl)carboxamido)cyclohexyl)ethyl)-2,3,4,5-tetrahydro-1H-3-benzazepine;
trans-7-(3-(5-Methyl)-1,2,4-oxadiazolyl)-3-(2-(1-(4-(3-(3-(5-Methyl)-1,2,4-oxadiazolyl)benzoyl)amino)cyclohexyl)ethyl)-2,3,4,5-tetrahydro-1H-3-benzazepine;
trans-(E)-7-(3-(5-Methyl)-1,2,4-oxadiazolyl)-3-(2-(1-(4-(3-(3-fluoro)phenylpropenoyl)amino)cyclohexyl)ethyl)-2,3,4,5-tetrahydro-1H-3-benzazepine;
trans-(E)-7-(3-(5-Methyl)-1,2,4-oxadiazolyl)-3-(2-(1-(4-(3-(2-fluoro)phenylpropenoyl)amino)cyclohexyl)ethyl)-2,3,4,5-tetrahydro-1H-3-benzazepine;
trans-7-(5-(3-Methyl)isoxazolyl)-3-(2-(1-(4-(2-fluoro)phenylacetamido)cyclohexyl)ethyl)-2,3,4,5-tetrahydro-1H-3-benzazepine;
trans-7-(3-(5-Methyl)isoxazolyl)-3-(2-(1-(4-(4-fluoro)phenylacetamido)cyclohexyl)ethyl)-2,3,4,5-tetrahydro-1H-3-benzazepine;
trans-(E)-7-(2-(5-Methyl)oxazolyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(3-(5-Methyl)isoxazolyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(5-Pyrimidyl)-3-(2-(1-(4-(3-(4-fluoro)phenylpropenoyl)amino)cyclohexyl)ethyl)-2,3,4,5-tetrahydro-1H-3-benzazepine;
trans-7-(5-Pyrimidinyl)-3-(2-(1-(4-(3-(3-(5-Methyl)-1,2,4-oxadiazolyl)benzoyl)amino)cyclohexyl)ethyl)-2,3,4,5-tetrahydro-1H-3-benzazepine;
trans-7-(3-(5-Methyl)isoxazolyl)-3-(2-(1-(4-(5-(2-methyl)quinolinyl)carboxamido)cyclohexyl)ethyl)-2,3,4,5-tetrahydro-1H-3-benzazepine; and
trans-3-(2-(1-(4-(3-(2-(5-Methyl)oxazolyl)benzoyl)amino)cyclohexyl)ethyl)-7-methylsulphonyloxy-2,3,4,5-tetrahydro-1H-3-benzazepine.
   Preferred Compounds include:
trans-7-Cyano-3-(2-(1-(4-(3-pyrrolo[2,3-b]pyridyl)carboxamido)cyclohexyl)ethyl)-2,3,4,5-tetrahydro-1H-3-benzazepine;
trans-7-Cyano-3-(2-(1-(2-naphthylacetamido)cyclohexyl)ethyl)-2,3,4,5-tetrahydro-1H-3-benzazepine;
trans-(E)-7-Cyano-3-(2-(1-(4-(3-(2-fluoro)phenylpropenoyl)amino)cyclohexyl)ethyl)-2,3,4,5-tetrahydro-1H-3-benzazepine;
trans-(E)-7-Cyano-3-(2-(1-(4-(3-(3-fluoro)phenylpropenoyl)amino)cyclohexyl)ethyl)-2,3,4,5-tetrahydro-1H-3-benzazepine;
trans-7-Cyano-3-(2-(1-(4-(8-(1,4-dihydro-4-oxo)quinolinyl)carboxamido)cyclohexyl)ethyl)-2,3,4,5-tetrahydro-1H-3-benzazepine;
trans-7-Cyano-3-(2-(1-(4-(2-naphthyl)carboxamido)cyclohexyl)ethyl)-2,3,4,5-tetrahydro-1H-3-benzazepine;
trans-7-Cyano-3-(2-(1-(4-(2-naphthyl)carboxamido)cyclohexyl)ethyl)-2,3,4,5-tetrahydro-1H-3-benzazepine;
trans-(E)-7-Cyano-3-(2-(1-(4-(3-(2-methoxy)phenylpropenoyl)amino)cyclohexyl)ethyl)-2,3,4,5-tetrahydro-1H-3-benzazepine;
trans-(E)-7-Cyano-3-(2-(1-(4-(3-(3-methoxy)phenylpropenoyl)amino)cyclohexyl)ethyl)-2,3,4,5-tetrahydro-1H-3-benzazepine;
trans-7-Cyano-3-(2-(1-(4-(7-(1,2-dihydro-2-oxo)quinolinyl)carboxamido)cyclohexyl)ethyl)-2,3,4,5-tetrahydro-1H-3-benzazepine;
trans-7-Cyano-3-(2-(1-(4-(3-(3-(5-ethyl)-1,2,4-oxadiazolyl)benzoyl)amino)cyclohexyl)ethyl)-2,3,4,5-tetrahydro-1H-3-benzazepine;
trans-7-Cyano-3-(2-(1-(4-(1-naphthyl)acetamido)cyclohexyl)ethyl)-2,3,4,5-tetrahydro-1H-3-benzazepine;
trans-7-Cyano-3-(2-(1-(4-(6-(2,3-dihydro-2-oxo)indolyl)acetamido)cyclohexyl)ethyl)-2,3,4,5-tetrahydro-1H-3-benzazepine;
trans-7-Cyano-3-(2-(1-(4-(5-(2,3-dihydro-2-oxo)indolyl)acetamido)cyclohexyl)ethyl)-2,3,4,5-tetrahydro-1H-3-benzazepine;
trans-7-Cyano-3-(2-(1-(4-(6-(1,2-dihydro-2-oxo)quinolinyl)acetamido)cyclohexyl)ethyl)-2,3,4,5-tetrahydro-1H-3-benzazepine;
trans-7-Cyano-3-(2-(1-(4-(7-(1,2-dihydro-2-oxo)quinolinyl)acetamido)cyclohexyl)ethyl)-2,3,4,5-tetrahydro-1H-3-benzazepine;
trans-7-Cyano-3-(2-(1-(4-(6-quinoxalinyl)acetamido)cyclohexyl)ethyl)-2,3,4,5-tetrahydro-1H-3-benzazepine;
trans-7-Cyano-3-(2-(1-(4-(6-(3,4-dihydro-2-oxo)-2H-benzoxazinyl)acetamido)cyclohexyl)ethyl)-2,3,4,5-tetrahydro-1H-3-benzazepine;
trans-(E)-7-Cyano-3-(2-(1-(4-(3-(2-fluoro-5-acetamido)phenylpropenoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(5-(2-methyl)quinolinyl)carboxamido)cyclohexyl)ethyl)-2,3,4,5-tetrahydro-1H-3-benzazepine;
trans-3-(2-(1-(4-(3-(2-(4-Methyl)oxazolyl)benzoyl)amino)cyclohexyl)ethyl)-7-methylsulphonyl-2,3,4,5-tetrahydro-1H-3-benzazepine;
trans-3-(2-(1-(4-(3-trifluoromethylbenzoyl)amino)cyclohexyl)ethyl)-7-methylsulphonyl-2,3,4,5-tetrahydro-1H-3-benzazepine;
trans-7-(5-Pyrimidinyl)-3-(2-(1-(4-(5-(2-methyl)quinolinyl)carboxamido)cyclohexyl)ethyl)-2,3,4,5-tetrahydro-1H-3-benzazepine;
trans-(E)-7-(2-Pyridyl)-3-(2-(1-(4-(3-(2-cyano)phenylpropenoyl)amino)cyclohexyl)ethyl)-2,3,4,5-tetrahydro-1H-3-benzazepine;
trans-(E)-7-(2-Pyridyl)-3-(2-(1-(4-(3-(3-cyano)phenylpropenoyl)amino)cyclohexyl)ethyl)-2,3,4,5-tetrahydro-1H-3-benzazepine; and
trans-(E)-7-(2-Pyridyl)-3-(2-(1-(4-(3-(4-cyano)phenylpropenoyl)amino)cyclohexyl)ethyl)-2,3,4,5-tetrahydro-1H-3-benzazepine.
   Most preferred compounds include:
trans-(E)-7-Cyano-3-(2-(1-(4-(5-quinolinyl)carboxamido)cyclohexyl)ethyl)-2,3,4,5-tetrahydro-1H-benzazepine;
trans-7-Cyano-3-(2-(1-(4-(5-(8-fluoro)quinolinyl)carboxamido)cyclohexyl)ethyl)-2,3,4,5-tetrahydro-1H-3-benzazepine;
trans-3-(2-(1-(4-(5-(2-Methyl)quinolinyl)carboxamido)cyclohexyl)ethyl)-7-methylsulphonyl-2,3,4,5-tetrahydro-1H-3-benzazepine;
trans-3-(2-(1-(4-(5-(2-Methyl)quinolinyl)carboxamide)cyclohexyl)ethyl)-7-methanesulphonyloxy-2,3,4,5-tetrahydro-1H-3-benzazepine;
trans-(E)-3-(2-(1-(4-(3-(4-fluoro)phenylpropenoyl)amino)cyclohexyl)ethyl)-7-methylsulphonyl-2,3,4,5-tetrahydro-1H-3-benzazepine;
trans-3-(2-(1-(4-(5-(8-Chloro-2-methyl)quinolinyl)carboxamide)cyclohexyl)ethyl)-7-methanesulphonyl-2,3,4,5-tetrahydro-1H-3-benzazepine;
trans-(E)-7-(2-Pyridyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(2-Pyrimidyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(1-Pyrrolidinylcarbonyl)-3-(2-(1-(4-(3-(4-fluoro)phenylpropenoyl)amino)cyclohexyl)ethyl)-2,3,4,5-tetrahydro-1H-3-benzazepine;
trans-7-(1-Pyrrolidinylcarbonyl)-3-(2-(1-(4-(3-pyrrolo[2,3-b]pyridyl)carboxamido)cyclohexyl)ethyl)-2,3,4,5-tetrahydro-1H-3-benzazepine;
trans-3-(2-(1-(4-(5-(8-Fluoro-2-methyl)quinolinyl)carboxamido)cyclohexyl)ethyl)-7-methylsulphonyl-2,3,4,5-tetrahydro-1H-3-benzazepine;
trans-3-(2-(1-(4-(5-(8-Fluoro-2-methyl)quinolinyl)carboxamido)cyclohexyl)ethyl)-7-methylsulphonyloxy-2,3,4,5-tetrahydro-1H-3-benzazepine.

These compounds may be in the form of their free base or physiologically acceptable salts thereof, particularly the monohydrochloride or monomesylate salts. The present invention also provides a process for preparing compounds of formula (I) which process comprises:

(a) reacting a compound of formula (II): 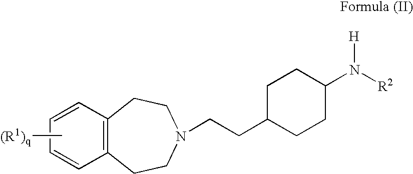
wherein R¹, R² and q are as hereinbefore defined, with a compound of formula (III):
A-COX Formula (III)
wherein A is as hereinbefore defined and X is a halogen atom or the residue of an activated ester;

(b) to prepare a compound of formula (I) by reacting a compound of formula (II) with a compound A-Br, or A-I, or A-OSO₂CF₃ in the presence of carbon monoxide and a catalyst such as trans-bis-triphenylphosphinepalladium(II)bromide;

(c) to prepare a compound of formula (I) wherein R¹ is Ar³-Z and Z is a bond, reacting a compound of formula (IV): 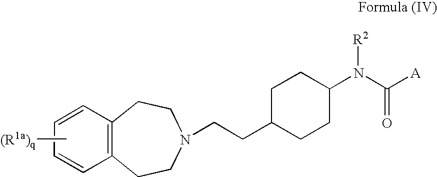
wherein R² and A are as hereinbefore defined and one R^{1a} represents a group W wherein W is a halogen atom or a trifluoromethylsulfonyloxy group, or W is a group M selected from a boron derivative e.g. a boronic acid function B(OH)₂ or a metal function such as trialkylstannyl e.g. SnBu₃, zinc halide or magnesium halide, and when q is 2 the other R^{1a} is R¹; with a compound Ar³—W¹, wherein W¹ is a halogen atom or a trifluoromethylsulfonyloxy group when W is a group M or W¹ is a group M when W is a halogen atom or a trifluoromethylsulfonyloxy group;

(d) to prepare a compound of formula (I) wherein R¹ is Ar³-Z and Z is O or S, reacting a compound of formula (V): 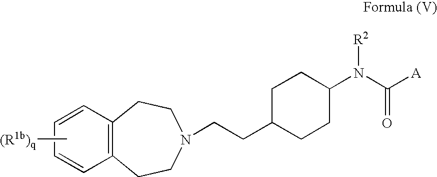
wherein R² and A are as hereinbefore defined and one R^{1b} represents a group ZH and when q is 2 the other R^{1b} represents R¹; with a reagent serving to introduce the group Ar³;

(e) to prepare a compound of formula (I) where Y is a bond, reaction of a compound of formula (VI): 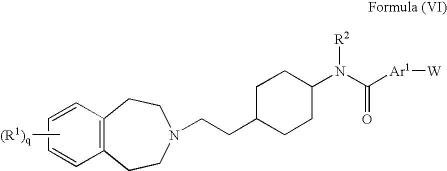
wherein R¹, R², Ar¹, W and q are as hereinbefore defined, with a compound Ar²—W¹, wherein W¹ is a halogen atom or a trifluoromethylsulfonyloxy group when W is a group M, or W¹ is a group M when W is a halogen atom or a trifluoromethylsulfonyloxy group.

(f) interconversion of one compound of formula (I) to a different compound of formula (I) e.g. (i) alkylation of a compound (I) wherein R² represents hydrogen, (ii) conversion of one R¹ from alkoxy (e.g. methoxy) to hydroxy, or (iii) conversion of R¹ from hydroxy to sulfonyloxy, eg alkylsulfonyloxy or trifluoromethanesulfonyloxy; (iv) conversion of a compound wherein Y represents S to a compound wherein Y is SO₂ or (v) conversion of Y from CO to CH₂;

(g) separation of cis- and trans-isomers of compounds of formula (I) by conventional methods, e.g. chromatography or crystallisation; and optionally thereafter forming a salt of formula (I).

Process (a) may be effected using conventional methods for the formation of an amide bond. When X is the residue of an activated ester this may be formed with e.g. a carbodiimide such as 1-ethyl-3-(3-dimethylaminopropyl)carbodiimide. The reaction may be carried out in a solvent such as dichloromethane.

Reaction of a compound of formula (IV) with Ar³W¹, according to process (c) or a compound of formula (VI) with Ar²—W¹ according to process (e) may be effected in the presence of a transition metal eg palladium catalyst such as bis-triphenylphosphinepalladium dichloride or tetrakis-triphenylphosphinepalladium (0). When M represents a boronic acid function such as B(OH)₂ the reaction may be carried out under basic conditions, for example using aqueous sodium carbonate in a suitable solvent such as dioxane. When M is trialkylstannyl the reaction may be carried out in an inert solvent, such as xylene or dioxane optionally in the presence of LiCl. When M is a zinc or magnesium halide the reaction may be effected in an aprotic solvent such as tetrahydrofuran. The substituent W is preferably a halogen atom such as bromine, or a sulfonyloxy group such as trifluoromethylsulfonyloxy; and W¹ is preferably a group M, such as trialkylstannyl or B(OH)₂.

In process (d) the reagent serving to introduce the group Ar³ is preferably a compound of formula Ar³-Hal, wherein Hal is a halogen atom. The reaction may be effected in the presence of a base, such as potassium carbonate, in a solvent such as dimethylformamide.

Interconversion reactions according to process (f) may be effected using methods well known in the art.

Compounds of formula (II) may be prepared by conversion of a compound of formula (VII), wherein R¹ and q are as hereinbefore defined, 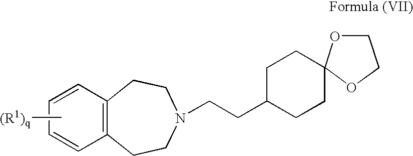
into a corresponding ketone, followed by reductive amination. This may be effected by methods well known in the art for (i) conversion of a ketal to a ketone in the presence of aqueous acid; followed by (ii) reductive amination of the ketone with R²NH₂ or ammonium acetate in the presence of a reducing agent. Suitable reducing agents which may be employed include sodium borohydride, cyanoborohydride or triacetoxyborohydride under acidic conditions, or catalytic hydrogenation. The reaction may conveniently be effected in a solvent such as methanol, ethanol or dichloroethane.

A compound of formula (VII) may itself be prepared by reacting a compound of formula (VIII): 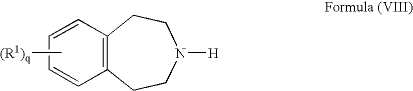
wherein R¹ and q are as hereinbefore defined;

with a compound of formula (IX): 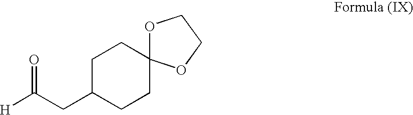
in the presence of a reducing agent. Suitable reducing agents which may be employed include sodium borohydride, cyanoborohydride or triacetoxyborohydride under acidic conditions, or catalytic hydrogenation. The reaction may conveniently be effected in a solvent such as ethanol or dichloroethane.

The individual cis- and trans-isomers of a compound of formula (II) may be prepared starting from cis- or trans-4-amino-cyclohexaneacetic acid (T. P. Johnson, et al., J. Med. Chem., 1997, (20), 279-290) followed by functional group interchange and/or protection using methods well known in the art, to give the individual cis- or trans-isomers of a compound of formula (X): 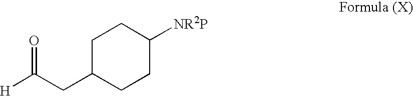
wherein R² is as hereinbefore defined, and P is a protecting group, for example trifluoroacetyl or tert-butoxycarbonyl. Subsequent reaction of a compound of formula (X) with a compound of formula (VIII) in the presence of a reducing agent as described above followed by deprotection using standard methodology gives the individual isomers of a compound of formula (II) wherein R² is as hereinbefore defined.

Compounds of formula (III) are known or may be prepared using standard procedures.

Compounds of formula (IV), (V) or (VI) may be prepared by processes analogous to (a), (b), (c) and (d) described above. Compounds Ar²W¹, Ar³W¹ and Ar³Hal are commercially available or may be prepared by standard methods. Compounds of formula (VIII), where for example R¹ is a halogen, methoxy, acetyl, cyano, carboxylic acid or carboxamide group are known in the literature or may be prepared by known methods. The compound of formula (IX) is likewise known in the literature.

Conversion of a compound of formula (VIII) where R¹ is a cyano or acetyl group to a compound of formula (VIII) where R¹ is a group Ar³Z, where Ar is an oxadiazole or an isoxazole ring and Z is a bond, may be carried out by (i) conversion to a compound of formula (XI), where R¹ and q are as hereinbefore defined, using standard methods; (ii) conversion of R¹ from cyano to oxadiazolyl using known methods, or conversion of acetyl to isoxazolyl using known methods; (iii) deprotection of a compound of formula (XI) to a compound of formula (VIII) using standard methods.

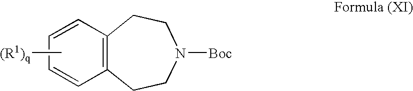

The invention is further illustrated by the following non-limiting examples:

### DESCRIPTION 1

### 2,3,4,5-Tetrahydro-1H-3-benzazepine

1,2-Phenylenediacetonitrile (7.5 g, 48 mmol) dissolved in ethanol (150 ml) was added to Raney Ni (2 g) which had been previously washed with ethanol (3×20 ml). The mixture was then hydrogenated at 50° C. at 50 psi pressure with shaking for 24 h. The reaction mixture was then cooled to room temperature and filtered through a pad of kieselguhr and washed through with ethanol (100 ml). The filtrate was evaporated in vacuo to give a brown oil which was chromatographed on silica gel (100 g), eluting with 2-10% methanol in CH₂Cl₂ to give the title compound as a brown oil (2.45 g, 35%).

Mass spectrum (API⁺) Found: 148 (MH⁺). C₁₀H₁₃N requires 147.

### DESCRIPTION 2

### trans-3-(2-(1-(4-(N-tert-Butoxycarbonyl)amino)cyclohexyl)ethyl)-2,3,4,5-tetrahydro-1H-3-benzazepine

Sodium triacetoxyborohydride (4.3 g, 20.4 mmol) was added to a mixture of 2,3,4,5-tetrahydro-1H-3-benzazepine (2.0 g, 13.6 mmol), and trans-2-(1-(4-(N-tert-butoxycarbonyl)amino)cyclohexyl)acetaldehyde in 1,2-dichloroethane (200 ml), and the mixture stirred at room temperature for 0.5 h. The reaction mixture was diluted with CH₂Cl₂ (100 ml) and washed with saturated aqueous K₂CO₃ (200 ml), followed by brine (100 ml). The organic layer was separated and dried over Na₂SO₄, then evaporated in vacuo to give an off-white solid which was chromatographed on silica gel eluting with ethyl acetate to give the title compound as an off-white solid (3.13 g, 62%).

Mass spectrum (API⁺): Found 373. C₂₃H₃₆N₂O₂ requires 372.

The following compound was prepared in a similar manner to Description 2

### (a) trans-3-(2-(1-(4-N-tert-Butoxycarbonyl)amino)cyclohexyl)ethyl-6-methoxy-2,3,4,5-tetrahydro-1H-3-benzazepine

Mass spectrum (API⁺): Found 403 (MH⁺). C₂₄H₃₈N₂O₃ requires 402.

### b) trans-3-(2-(1-(4-N-tert-Butyloxycarbonyl)amino)cyclohexyl)ethyl-7-cyano-2,3,4,5-tetrahydro-1H-3-benzazepine

Mass spectrum (API⁺) Found 398 (MH⁺). C₂₄H₃₅N₃O₂ requires 397.

¹H NMR (CDCl₃) δ: 0.97-1.13 (4H, m), 1.22 (1H, m), 1.36-1.47 (11H, m), 1.71-1.79 (2H, m), 1.95-2.04 (2H, m), 2.48 (2H, m), 2.61 (4H, m), 2.90-3.00 (4H, m), 3.37 (1H, m), 4.35 (1H, m), 7.17 (1H, d, J=5 Hz), 7.36 (1H, s), 7.52 (1H, d, J=5 Hz).

### DESCRIPTION 3

### trans-3-(2-(1-(4-Amino)cyclohexyl)ethyl)-2,3,4,5-tetrahydro-1H-3-benzazepine

A mixture of trans-2-(2-(1-(4-(N-tert-butoxcarbonyl)amino)cyclohexyl)ethyl)-2,3,4,5-tetrahydro-1H-3-benzazepine (3.1 g, 8.3 mmol) and trifluoroacetic acid (5 ml) in CH₂Cl₂ (50 ml) was stirred at room temperature for 1 h, then at 40° C. for 1 h. The reaction mixture was then diluted with CH₂Cl₂ (100 ml) and washed with saturated aqueous K₂CO₃ (2×100 ml). The organic layer was dried over Na₂SO₄ and evaporated in vacuo to give the title compound as a brown oil (2.14 g, 95%).

Mass spectrum (API⁺): Found 273 (MH⁺). C₁₈H₂₈N₂ requires 272.

### The Following Compound was Prepared in a Similar Manner to Description 3

### (a) trans-3-(2-(1-(4-Amino)cyclohexyl)ethyl)-6-methoxy-2,3,4,5-tetrahydro-1H-3-benzazepine

Mass spectrum (API⁺): Found 303 (MH⁺). C₁₉H₃₀N₂O requires 302.

### b) trans-3-(2-(1-(4-Amino)cyclohexyl)ethyl)-7-cyano-2,3,4,5-tetrahydro-1H-3-benzazepine

Mass spectrum (API⁺): Found 298 (MH⁺). C₁₉H₂₇N₃ requires 297.

¹H NMR (CDCl₃) δ: 0.92-1.18 (6H, m), 1.21 (1H, m), 1.41 (2H, m), 1.75 (2H, m), 1.85 (2H, m), 2.49 (2H, m), 2.60 (5H, m), 2.95 (4H, m), 7.16 (1H, d, J=5 Hz), 7.36 (1H, s), 7.40 (1H, d, J=5 Hz).

### DESCRIPTION 4

### trans-2-(1-(4-(N-tert-Butyloxycarbonyl)amino)cyclohexyl)acetic acid, methyl ester

A mixture of trans-(4-amino)cyclohexylactic acid hydrogen sulfate (T. P. Johnston et al; J. Med Chem., 1977, 20 (2), 279-290), (27.0 g, 106 mmol), conc. H₂SO₄ (3 ml), and methanol (300 ml) was stirred at reflux for 5 h. Resulting solution was filtered and the filtrate evaporated in vacuo to give a brown oil (36 g). A mixture of this material, triethylamine (36 ml; 26.1 g, 259 mmol), dichloromethane (600 ml) and di-t-butyl dicarbonate (25.5 g, 117 mmol) was stirred at 20° C. for 18 h. Resulting solution was partitioned between saturated aqueous NaHCO₃ (500 ml) and dichloromethane (3×200 ml), and the combined extracts were dried (Na₂SO₄) and evaporated in vacuo to give the title compound (24.6 g, 86%) as a colourless solid.

¹H NMR (CDCl₃) δ: 1.08 (4H, m), 1.43 (9H, s), 1.76 (3H, m), 2.00 (2H, m), 2.20 (2H, d, J=7 Hz), 3.37 (1H, m), 3.66 (3H, s), 4.39 (1H, br s).

### DESCRIPTION 5

### trans-2-(1-(4-(N-tert-Butyloxycarbonyl)amino)cyclohexyl)acetaldehyde

To a stirred solution of trans-2-(1-(4-(N-tert-butyloxycarbonyl)amino)cyclohexyl)acetic acid, methyl ester (46.0 g, 170 mmol) in dry toluene (920 ml) at −78° C. under argon was added a solution of di-isobutylaluminium hydride (1M; 285 ml; 285 mmol), dropwise over 0.5 h. Resulting solution was stirred for a further 0.3 h and quenched with a mixture of methanol (28 ml) in toluene (50 ml) and then poured into saturated aqueous potassium sodium tartrate (1.2 L). The resultant mixture was extracted with ether (4×1 L). The combined organic extracts were dried (Na₂SO₄) and evaporated in vacuo to give a waxy solid which was purified using silica gel, eluting with 10-50% ethyl acetate/hexane to give the title compound (21.77 g, 53%) as a colourless solid.

¹H NMR (CDCl₃) δ: 1.12 (4H, m), 1.44 (9H, s), 1.78 (3H, m), 2.00 (2H, m), 2.33 (2H, dd, J=7, 2 Hz), 3.37 (1H, m), 4.40 (1H, m), 9.75 (1H, m).

### DESCRIPTION 6

### 7-Hydroxy-2,3,4,5-tetrahydro-1H-3-benzazepine, hydrobromide

7-Methoxy-2,3,4,5-tetrahydro-1H-3-benzazepine (10 g) in 48% aqueous hydrobromic acid (350 ml) was allowed to stir at 100° C. for 4 h. The mixture was cooled to 20° C. then evaporated to dryness in vacuo to give the title compound (14.5 g) as a brown solid.

Mass spectrum (API⁺): Found 164 (MH⁺). C₁₀H₁₃NO requires 163.

¹H NMR (DMSO) δ: 2.80-3.25 (8H, m), 4.42 (2H, br s), 6.50-6.70 (2H, m), 6.98 (1H, d, J=8 Hz), 8.86 (1H, br s).

### DESCRIPTION 7

### 3-(tert-Butyloxycarbonyl)-7-hydroxy-2,3,4,5-tetrahydro-1H-3-benzazepine

To a solution of 7-hydroxy-2,3,4,5-tetrahydro-1H-3-benzazepine, hydrobromide (14.5 g) in tetrahydrofuran (100 ml) and water (70 ml), was added triethylamine (8 g), followed by a solution of di-tert-butyl dicarbonate (14 g) in THF (20 ml). The resulting mixture was allowed to stir at 20° C. for 16 h, partitioned between ethyl acetate (200 ml) and water (200 ml). The aqueous layer was washed with ethyl acetate (100 ml). The combined organic extracts were washed with saturated aqueous sodium bicarbonate (100 ml), dried (Na₂SO₄) and evaporated to dryness in vacuo. The resulting oil was purified by silica gel chromatography. Elution with ethyl acetate in hexane (10%-30%) gave the title compound (8 g).

Mass spectrum (API⁺): Found 164 (MH⁺-Boc). C₁₅H₂₁NO₃ requires 263.

¹H NMR (CDCl₃) δ: 1.48 (9H, s), 2.75-2.87 (4H, m), 3.40-3.60 (4H, m), 4.95 (1H, s), 6.50-6.62 (2H, m), 6.96 (1H, d, J=8 Hz).

### DESCRIPTION 8

### 3-(tert-Butyloxycarbonyl)-7-trifluoromethylsulfonyloxy-2,3,4,5-tetrahydro-1H-3-benzazepine

To a stirred mixture of 3-(tert-butyloxycarbonyl)-7-hydroxy-2,3,4,5-tetrahydro-1H-3-benzazepine (7 g) and triethylamine (5.4 ml) in dry dichloromethane under argon at −20° C., was added, dropwise, trifluoromethanesulfonic anhydride (5 ml). The resulting mixture was allowed to warm slowly to 20° C. over 16 h, then was poured into saturated aqueous sodium bicarbonate (200 ml) and extracted with dichloromethane (2×150 ml). The combined organic extracts were washed with brine (150 ml), dried (Na₂SO₄) and evaporated in vacuo to give an amber oil. Silica gel chromatography, eluting with ethyl acetate in hexane (10%-30%) gave the title compound (7 g) as an amber oil.

Mass spectrum (API⁺): Found 396 (MH⁺). C₁₆H₂₀F₃NO₅S requires 395.

¹H NMR (CDCl₃) δ: 1.48 (9H, s), 2.85-2.95 (4H, m), 3.5-3.65 (4H, m), 7.00-7.05 (2H, m), 7.15-7.27 (1H, m).

### DESCRIPTION 9

### 3-(tert-Butyloxycarbonyl)-7-cyano-2,3,4,5-tetrahydro-1H-3-benzazepine

A mixture of 3-(tert-butoxycarbonyl)-7-trifluoromethylsulfonyloxy-2,3,4,5-tetrahydro-1H-3-benzazepine (4.78 g, 12.1 mmol), zinc cyanide (1.42 g, 15.6 mmol) and tetrakis-triphenylphosphine palladium (0) (1.4 g, 1.2 mmol, 10 mol %), in dry dimethylformamide (50 ml) was stirred at 100° C. for 3 h under argon. After cooling to room temperature the reaction mixture was diluted with ethyl acetate (120 ml) and filtered. The filtrate was washed with saturated aqueous sodium bicarbonate (100 ml), then water (2×50 ml), then brine (50 ml). The organic layer was dried over sodium sulfate and evaporated in vacuo to give brown oil, which was purified by chromatography on silica gel with 20-100% ethyl acetate-hexane elution to give the title compound (0.765 g, 23%) as a brown oil.

Mass spectrum (API⁺): Found 173 (MH⁺-Boc). C₁₆H₂₀N₂O₂ requires 272.

¹H NMR (CDCl₃) δ: 1.47 (9H, s), 2.93 (4H, m), 3.56 (4H, m), 7.21 (1H, d, J=8 Hz), 7.42 (2H, m).

### DESCRIPTION 10

### 7-Cyano-2,3,4,5-tetrahydro-1H-3-benzazepine

A mixture of 3-(tert-butoxycarbonyl)-7-cyano-2,3,4,5-tetrahydro-1H-3-benzazepine (765 mg, 2.81 mmol) and trifluoroacetic acid (2 ml), in dichloromethane (20 ml) was stirred at 40° C. for 1 h. The reaction mixture was evaporated to dryness in vacuo and partitioned between ethyl acetate (50 ml) and water (50 ml). The aqueous layer was basified using potassium carbonate and re-extracted with ethyl acetate (2×30 ml). The combined basic organic extracts were dried over sodium sulfate and evaporated in vacuo to give the title compound as a colourless oil (212 mg, 44%).

Mass spectrum (API⁺): Found 173 (MH⁺). C₁₁H₁₂N₂ requires 172.

¹H NMR (CDCl₃) δ: 2.04 (1H, br s), 2.95 (8H, m), 7.18 (1H, d, J=8 Hz), 7.38 (2H, m).

### DESCRIPTION 11

### 3-(3-Bromophenyl)-5-methyl-1,2,4-oxadiazole

Potassium tert-butoxide (7.33 g, 65.4 mmol) was added over 5 minutes to ice chilled, stirred methanol under argon. After a further 5 min hydroxylamine hydrochloride (4.9 g, 70.43 mmol) was added in one portion and the resultant mixture stirred at room temperature for 1 h. A solution of 3-bromobenzonitrile (7.93 g, 43.6 mmol) in methanol (120 ml) was added in one portion and the mixture heated at reflux for 4 h, cooled filtered, and the filtrate evaporated in vacuo. The residue was refluxed in acetic anhydride (60 ml) for 3 h, cooled to room temperature and poured into ice-water (300 ml). The precipitate was filtered, washed with water, dried in vacuo and chromatographed on silica eluting with 0-10% ethyl acetate-hexane gradient. Fractions containing desired product were pooled and evaporated in vacuo and the residue recrystallised from hexane to afford the title compound as colourless crystals (5.2 g, 50%).

Mass spectrum: (API⁺) Found: 239 (MH⁺). C₉H₇⁷⁹BrN₂O requires 238

¹H NMR (CDCl₃) δ: 2.66 (3H, s), 7.36 (1H, t, J=8 Hz), 7.63 (1H, m), 8.05 (1H, m), 8.23 (1H, m).

### DESCRIPTION 12

### 3-(5-Methyl-1,2,4-oxadiazol-3-yl)-benzoic acid

A mixture of 3-(3-bromophenyl)-5-methyl-1,2,4-oxadiazole (2.68 g, 11.3 mmol), tributylamine (3.05 ml, 12.5 mmol) and trans-dibromobis(triphenylphosphine)palladium (II) (0.13 g, 0.16 mmol) in methanol (5 ml) was carbonylated at 30 psi and 100° C. for 18 h. The mixture was cooled to room temperature, diluted with ethyl acetate (100 ml) and washed sequentially with saturated sodium hydrogen carbonate (2×300 ml), brine (100 ml), 0.5 N hydrochloric acid (200 ml), brine (100 ml), then dried Na₂SO₄) and evaporated in vacuo to afford a yellow oil (2.49 g). A 2 g sample of this oil was dissolved in aqueous methanol (5:3, 80 ml), sodium hydroxide (0.36 g) added and the mixture stirred at room temperature for 20 h. The mixture was evaporated in vacuo and the residue partitioned between ethyl acetate (100 ml) and water (100 ml). The aqueous layer was acidified with 2N HCl and the resultant precipitate filtered, washed with water and dried in vacuo to afford the title compound as a colourless solid (0.78 g, 42%).

Mass spectrum: (API⁺) Found: 205 (MH⁺). C₁₀H₈N₂O₃ requires 204.

¹H NMR (CDCl₃) δ: 2.70 (3H, s), 7.71 (1H, m), 8.14 (1H, dd, J=7,1 Hz), 8.23 (1H, dd, J=7, 1 Hz), 8.54 (1H, m), 13.35 (1H, br s).

### DESCRIPTION 13

### 3-(1-Pyrazolyl)-benzoic acid

A mixture of 3-hydrazinobenzoic acid (1.52 g, 0.01 mmol) and malondialdehydebis(dimethylacetal) (2.39 ml; 0.01 mol) in ethanol (10 ml) and water (15 ml) was heated at reflux for 2 h. The resulting solution was cooled and evaporated to afford the title product (1.8 g,; 96%) as a yellow solid.

¹H NMR (DMSO-d₆) δ: 6.60 (1H, t, J=2 Hz), 7.65 (1H, t, J=8 Hz), 7.81 (1H, d, J=1.5 Hz), 7.89 (1H, dd, J=8 and 1.5 Hz), 8.12 (1H, dd, J=8 and 1.5 Hz), 8.4 (1H, d, J=2 Hz), 8.64 (1H, d, J=2 Hz).

Mass spectrum (API⁺): Found 189 (MH⁺). C₁₀H₈N₂O₂ requires 188.

### DESCRIPTION 14

### 3-(tert-Butoxycarbonyl)-7-(3-(5-methyl)-1,2,4-oxadiazolyl)-2,3,4,5-tetrahydro-1H-3-benzazepine

To a suspension of sodium methoxide (0.6 g, 11 mmol) in anhydrous methanol (12 ml) under argon, was added hydroxylamine hydrochloride (0.76 g, 11 mmol), followed by 3-(tert-butyloxycarbonyl)-7-cyano-2,3,4,5-tetrahydro-1H-3-benzazepine (1.5 g, 5.5 mmol). The mixture was stirred under reflux for 16 h, then allowed to cool to room temperature. The methanol was evaporated in vacuo and the resulting residue partitioned between dichloromethane (100 ml) and water (100 ml). The aqueous layer was washed with more CH₂Cl₂ (100 ml). The combined organic extracts were dried and evaporated in vacuo to give a solid (1.8 g), which was mixed with acetic anhydride (15 ml) and heated at 120° C. for 2 h. Excess acetic anhydride was evaporated in vacuo and the resulting oily residue partitioned between CH₂Cl₂ (250 ml) and saturated sodium bicarbonate solution (250 ml). The organic layer was washed with more bicarbonate solution (200 ml), dried, and evaporated to give an oil. Gravity silica gel chromatography eluting with ethyl acetate in hexane gave the title compound (3.2 g, 73%) as a colourless oil.

¹H NMR (CDCl₃) δ: 1.49 (9H, s), 2.65 (3H, s), 2.96 (4H, m), 3.58 (4H, m), 7.22 (1H, d, J=8 Hz), 7.80 (2H, m).

### DESCRIPTION 15

### 7-(3-(5-Methyl)-1,2,4-oxadiazolyl)-2,3,4,5-tetrahydro-1H-3-benzazepine

A solution of 3-(tert-butoxycarbonyl)-7-(3-(5-methyl)-1,2,4-oxadiazolyl)-2,3,4,5-tetrahydro-1H-3-benzazepine (1.2 g, 3.6 mmol) in CH₂Cl₂ (15 ml) and trifluoroacetic acid (15 ml) was heater under reflux for 2 h. Solvent was evaporated in vacuo and the residue partitioned between diethyl ether (50 ml) and water (50 ml). The aqueous layer was saturated with potassium carbonate then extracted with CH₂Cl₂ (2×100 ml). The combined organic extracts were dried and evaporated in vacuo to give the title compound (0.74 g, 88%) as an oil.

Mass spectrum (API⁺): Found 230 (MH⁺). C₁₃H₁₅N₃O requires 229.

¹H NMR (CDCl₃) δ: 1.80 (1H, br s), 2.65 (3H, s), 2.90-3.00 (8H, m), 7.20 (1H, d, J=8 Hz), 7.75-7.85 (2H, m).

### DESCRIPTION 16

### 7-(3-(tert-Butoxycarbonyl)-2,3,4,5-tetrahydro-1H-3-benzazepinyl)carboxamide

To a solution of 3-tert-butyloxycarbonyl)-7-cyano-2,3,4,5-tetrahydro-1H-3-benzazepine (5.44 g, 20 mmol) cooled in ice bath, was added potassium carbonate (0.4 g) in water (1 ml), followed by dropwise addition of 30% w/w hydrogen peroxide (2.4 ml). The resulting mixture was stirred at 5° C. for 5 min, then the ice-bath was removed. After another 5 min, water (100 ml) was added. The solid precipitate was collected by filtration and dried to give the title compound (4.35 g, 75%) as a colourless solid.

¹H NMR (CDCl₃) δ: 1.48 (9H, s), 2.96 (4H, m), 3.56 (4H, m), 5.60-6.30 (2H, br d), 7.19 (1H, d, J=8 Hz), 7.50-7.80 (2H, m).

### DESCRIPTION 17

### 3-(tert-Butoxycarbonyl)-7-(5-(3-methyl)-1,2,4-oxadiazolyl)-2,3,4,5-tetrahydro-1H-3-benzazepine

A mixture of 7-(3-tert-butoxycarbonyl)-2,3,4,5-tetrahydro-1H-3-benzazepinyl)carboxamide (4.29 g, 14.8 mmol) and N,N-dimethyl acetamide dimethyl acetal (6 ml, 41 mmol) was heated at 125° C. under argon. Methanol was removed from the reaction by means of a distillation condenser over 2 h. The reaction mixture was further evaporated in vacuo to give a thick brown oily residue. To this residue was added, in order, dioxan (10 ml), 5M sodium hydroxide (4 ml), hydroxylamine hydrochloride (1.4 g, 20 mmol) and 70% aqueous acetic acid (20 ml). The combined mixture was allowed to stir at room temperature for 15 min and then at 90° C. for 1 h. The mixture was treated with water (100 ml) and extracted with CH₂Cl₂ (2×150 ml). Combined organic extracts were washed with saturated sodium bicarbonate (100 ml), dried and evaporated in vacuo to give an oil. Gravity silica gel chromatography, eluting with ethyl acetate in hexane, gave the title compound (3.9 g, 80%) as a colourless solid.

¹H NMR (CDCl₃) δ: 1.49 (9H, s), 2.47 (3H, s), 2.98 (4H, m), 3.60 (4H, m), 7.27 (1H, d, J=8 Hz), 7.80-7.90 (2H, m).

### DESCRIPTION 18

### 7-(5-(3-Methyl)-1,2,4-oxadiazolyl)-2,3,4,5-tetrahydro-1H-3-benzazepine

A solution of 3-(tert-butoxycarbonyl)-7-(5-(3-methyl)-1,2,4-oxadiazolyl)-2,3,4,5-tetrahydro-1H-3-benzazepine (3.8 g, 11.6 mmol) in CH₂Cl₂ (50 ml) and trifluoroacetic acid (12 ml) was heated under reflux for 2 h. Solvent was evaporated in vacuo and the residue partitioned between diethyl ether (200 ml) and water (200 ml). The aqueous layer was saturated with potassium carbonate then extracted with CH₂Cl₂ (3×200 ml). The combined organic extracts were dried and evaporated in vacuo to give the title compound (2.4 g, 91%) as a colourless solid.

Mass spectrum (API⁺): Found 230 (MH⁺). C₁₃H₁₅N₃O requires 229.

¹H NMR (CDCl₃) δ: 1.86 (1H, br s), 2.47 (3H, s), 3.00 (8H, m), 7.25 (1H, d, J=8 Hz), 7.80-7.90 (2H, m).

### DESCRIPTION 19

### trans-3-(2-(1-(4-N-tert-Butyloxycarbonyl)amino)cyclohexyl)ethyl-7-(3-(5-methyl)-1,2,4-oxadiazolyl)-2,3,4,5-tetrahydro-1H-3-benzazepine

Prepared from 7-(3-(5-methyl)-1,2,4-oxadiazolyl)-2,3,4,5-tetrahydro-1H-3-benzazepine in a manner similar to Description 2, in 96% yield.

Mass spectrum (API⁺): Found 455 (MH⁺). C₂₆H₃₈N₄O₃ requires 454.

¹H NMR (CDCl₃) δ: 0.90-1.10 (4H, m), 1.15-1.25 (1H, m), 1.38-1.47 (11H, m), 1.73-1.85 (2H, m), 1.93-2.05 (2H, m), 2.40-2.55 (2H, m), 2.56-2.70 (7H, m), 2.90-3.05 (4H, m), 3.35 (1H, br s), 4.35 (1H, br s), 7.19 (1H, d, J=8 Hz), 7.75-7.85 (2H, m).

### DESCRIPTION 20

### trans-3-(2-(1-(4-N-tert-Butyoxycarbonyl)amino)cyclohexyl)ethyl-7-(5-(3-methyl)-1,2,4-oxadiazolyl)-2,3,4,5-tetrahydro-1H-3-benzazepine

Prepared from 7-(5-(3-methyl)-1,2,4-oxadiazolyl)-2,3,4,5-tetrahydro-1H-3-benzazepine in a manner similar to Description 2, in 94% yield.

Mass spectrum (API⁺): Found 455 (MH⁺). C₂₆H₃₈N₄O₃ requires 454.

¹H NMR (CDCl₃) δ: 0.95-1.10 (4H, m), 1.23 (1H, br s), 1.40-1.50 (11H, m), 1.70-1.85 (2H, m), 1.95-2.10 (2H, m), 2.46 (3H, s), 2.46-2.52 (2H, m), 2.60-2.70 (4H, m), 2.90-3.60 (4H, m), 3.35 (1H, m), 4.35 (1H, m), 7.23 (1H, d, J=8 Hz), 7.80-7.90 (2H, m).

### DESCRIPTION 21

### trans-3-(2-(1-(4-Amino)cyclohexyl)ethyl)-7-(3-(5-methyl)-1,2,4-oxadiazolyl)-2,3,4,5-tetrahydro-1H-3-benzazepine

Prepared from trans-3-(2-(1-(4-N-tert-butyloxycarbonyl)amino)cyclohexyl)ethyl-7-(3-(5-methyl)-1,2,4-oxadiazolyl)-2,3,4,5-tetrahydro-1H-3-benzazepine in a manner similar to Description 3, in 100% yield.

Mass spectrum (API⁺): found 355 (MH⁺). C₂₁H₃₀N₄O requires 354.

¹H NMR (CDCl₃) δ: 0.90-1.10 (4H, m), 1.40 (2H, br s), 1.12-1.25 (1H, m), 1.40-1.50 (2H, m), 1.70-1.80 (2H, m), 1.80-1.90 (2H, m), 2.40-2.50 (2H, m), 2.55-2.70 (8H, m), 2.90-3.00 (4H, m), 7.19 (1H, d, J=8 Hz), 7.75-7.85 (2H, m).

### DESCRIPTION 22

### trans-3-(2-(1-(4-Amino)cyclohexyl)ethyl)-7-(5-(3-methyl)-1,2,4-oxadiazolyl)-2,3,4,5-tetrahydro-1H-3-benzazepine

Prepared from trans-3-(2-(1-(4-N-tert-butyloxycarbonyl)amino)cyclohexyl)ethyl-7-(5-(3-methyl)-1,2,4-oxadiazolyl)-2,3,4,5-tetrahydro-1H-3-benzazepine in a manner similar to Description 3, in 100% yield.

Mass spectrum (API⁺): Found 355 (MH⁺). C₂₁H₃₀N₄O requires 354.

¹H NMR (CDCl₃) δ: 0.90-1.30 (5H, m), 1.37-1.50 (2H, m), 1.64 (2H, br s), 1.70-1.95 (4H, m), 2.46 (3H, s), 2.46-2.70 (7H, m), 2.90-3.10 (4H, m), 7.24 (1H, d, J=8 Hz), 7.80-7.90 (2H, m).

### DESCRIPTION 23

### 3-Acetyl-2,3,4,5-tetrahydro-1H-3-benzazepine

A solution of acetic anhydride (6.37 g, 0.062 mol) in dichloromethane (50 ml) was added dropwise to a stirred solution of 2,3,4,5-tetrahydro-1H-3-benzazepine (8.35 g, 0.057 mol) and triethylamine (8.7 ml) in dichloromethane (50 ml) at 0° C. under argon. After stirring at room temperature for 18 h, water (80 ml) was added and the organic layer separated. The organic layer was washed with 0.5 M hydrochloric acid (50 ml), saturated sodium bicarbonate solution (50 ml), water (50 ml) and then dried (Na₂SO₄). Evaporation of the solvent in vacuo gave the title compound (10.24 g, 95%) as a yellow oil which solidified on standing.

¹H NMR (CDCl₃) δ: 2.18 (3H, s), 2.85-3.00 (4H, m), 3.55-3.60 (2H, m), 3.72-3.80 (2H, m), 7.10-7.20 (4H, m).

Mass Spectrum AP⁺: Found 190 (MH⁺). C₁₂H₁₅NO requires 189.

### DESCRIPTION 24

### 3-Acetyl-7-chlorosulphonyl-2,3,4,5-tetrahydro-1H-3-benzazepine

A solution of 3-acetyl-2,3,4,5-tetrahydro-1H-3-benzazepine (4.0 g, 0.021 mol) in dichloromethane (25 ml) was added dropwise to a stirred solution of chlorosulphonic acid in dichloromethane (25 ml) at −70° C. under argon. After warming to room temperature, the reaction was stirred for 18 h before being quenched in ice/water (200 ml). The resulting mixture was extracted with ethyl acetate (3×100 ml), dried (Na₂SO₄) and the solvent evaporated in vacuo to give the title compound (2.74 g, 45%) as a pale yellow solid.

¹H NMR: δ (CDCl₃): 2.21 (3H, s), 3.0-3.10 (4H, m), 3.60-3.70 (2H, m), 3.74-3.80 (2H, m), 7.35-7.40 (1H, m), 7.80-7.85 (2H, m).

Mass spectrum AP⁺: Found 288 & 290 (MH⁺). C₁₂H₁₄NSO₂Cl requires 287 & 289.

### DESCRIPTION 25

### 3-Acetyl-7-methylsulphonyl-2,3,4,5-tetrahydro-1H-3-benzazepine

To a stirred solution of sodium sulphite (1.60 g, 12.6 mmol) and sodium hydrogen carbonate (1.14 g, 13.56 mmol) in water (25 ml) was added 7-3-acetyl-7-chlorosulfonyl-2,3,4,5-tetrahydro-1H-3-benzazepine (2.6 g, 9.04 mmol) in tetrahydrofuran (10 ml). The reaction mixture was then heated at 75° C. for 2 h, cooled to 30° C. and methyl iodide (2.8 ml, 45.20 mmol) added. After stirring at 50° C. for 24 h, the reaction mixture was cooled to room temperature and partitioned between water (50 ml) and ethyl acetate (100 ml). The aqueous layer was then separated and further extracted with ethyl acetate (2×80 ml). The combined organics were then dried (Na₂SO₄) and the solvent removed in vacuo to give the title compound (1.77 g, 73%) as a pale yellow solid.

¹H NMR (CDCl₃) 2.20 (3H, s), 2.99-3.05 (4H, m), 3.06 (3H, s), 3.61-3.64 (2H, m), 3.73-3.77 (2H, m), 7.32-7.37 (1H, m), 7.7-7.75 (2H, m).

Mass Spectrum AP⁺: Found 268 (MH⁺). C₁₃H₁₇NSO₃ requires 267.

### DESCRIPTION 26

### 7-Methylsulphonyl-2,3,4,5-tetrahydro-1H-3-benzazepine

A solution of 3-acetyl-7-methylsulphonyl-2,3,4,5-tetrahydro-1H-3-benzazepine (1.75 g, 6.55 mmol) in 5 M hydrochloric acid was heated at reflux for 18 h. The reaction mixture was then cooled to room temperature, basified to pH=12 with potassium carbonate and the solvent evaporated in vacuo. The solid residue was then extracted with ethyl acetate (5×60 ml) and the combined organics dried (Na₂SO₄). The solvent was then evaporated in vacuo to give the title compound (450 mg, 32%) as a pale yellow oil.

¹H NMR (CDCl₃) 1.88 (1H, br s), 2.95-3.13 (8H, m), 3.04 (3H, s), 7.25-7.30 (1H, d), 7.65-7.72 (2H, m).

Mass Spectrum AP⁺: Found 226 (MH⁺). C₁₁H₁₅NSO₂ requires 225.

### DESCRIPTION 27

### trans-3-(2-(1-(4-(N-tert-Butyloxycarbonyl)amino)cyclohexyl)ethyl-7-methylsulphonyl-2,3,4,5-tetrahydro-1H-3-benzazepine

A solution of 7-methylsulphonyl-2,3,4,5-tetrahydro-1H-3-benzazepine (1.0 g, 4.67 mmol) and trans-(1-(4-N-tert-butyloxycarbonyl)amino)cyclohexylacetaldehyde (0.8 g, 3.34 mmol) in dichloroethane (20 ml) was stirred at room temperature for 5 min before sodium triacetoxyborohydride (0.95 g, 4.49 mmol) was added in a single portion. After stirring at room temperature for 48 h, the reaction mixture was partitioned between water (50 ml) and dichloromethane (100 ml). The aqueous layer was separate, re-extracted with dichloromethane (2×50 ml) and the combined organic layers dried (Na₂SO₄). The solvent was then removed in vacuo to give a pale yellow solid which was purified by column chromatography (silica gel; ethyl acetate:methanol; 9:1) to give the title compound (1.35 g, 90%) as a colourless solid.

¹H NMR (CDCl₃): 0.99-1.14 (4H, m), 1.23-1.29 (1H, m), 1.41-1.46 (2H, m), 1.46 (9H, s), 1.73-1.79 (2H, m), 2.00-2.06 (2H, m), 2.50 (2H, t, J=7.6 Hz), 2.62-2.65 (4H, m), 2.99-3.02 (4H, m), 3.05 (3H, s), 3.38 (1H, br s), 4.38 (1H, br s), 7.27-7.30 (1H, d), 7.67-7.74 (2H, m).

Mass spectrum: AP⁺ Found: 351 ([M-BOC]H⁺). C₂₄H₃₈N₂SO₄ requires 450.

### DESCRIPTION 28

### trans-3-(2-(1-(4-Amino)cyclohexyl)ethyl)-7-methylsulphonyl-2,3,4,5-tetrahydro-1H-3-benzazepine

A solution of trans-3-(2-(1-(4-N-tert-butyloxycarbonyl)amino)cyclohexyl)ethyl-7-methylsulphonyl-2,3,4,5-tetrahydro-1H-3-benzazepine (1.3 g, 2.89 mmol) in dichloromethane (24 ml) and trifluoroacetic acid (6 ml) were stirred at room temperature for 2 h. The reaction mixture was then concentrated in vacuo and the residue partitioned between water (60 ml) and ethyl acetate (20 ml). The aqueous layer was separated, extracted with ethyl acetate (30 ml) and then basified to pH=14 with 40% sodium hydroxide. The oily suspension was then extracted with ethyl acetate (3×60 ml) and the combined organic layers dried (Na₂SO₄). The solvent was evaporated in vacuo to give the title compound (1.01 g, 100%) as an off-white solid.

¹H NMR (CDCl₃) δ: 0.90-1.12 (4H, m), 1.15-1.22 (1H, m), 1.35-1.40 (2H, m), 1.72-1.78 (2H, m), 1.82-1.90 (2H, m), 2.45-2.52 (2H, m), 2.55-2.62 (5H, m), 2.98-3.02 (4H, m), 3.04 (3H, s), 7.27 (1H, d, J=7.8 Hz), 7.56 (1H, s), 7.68 (1H, d).

Mass spectrum: AP⁺ 351 (MH⁺): C₁₉H₃₀N₂SO₂ requires 350.

### DESCRIPTION 29

### 3-(tert-Butyloxycarbonyl)-7-(5-(3-methyl)isoxazolyl)-2,3,4,5-tetrahydro-1H-3-benzazepine

A mixture of 7-acetyl-3-(tert-butyloxycarbonyl)-2,3,4,5-tetrahydro-1H-3-benzazepine (6.18 g, 21.4 mmol) and dimethylacetamide dimethylacetal (8 ml) was stirred at 125° C. Methanol by-product was removed by means of a Dean-Stark apparatus. After 8 h, excess dimethyl acetamide dimethyl acetal was evaporated in vacuo to give a thick oily residue. Absolute ethanol (20 ml) and hydroxylamine hydrochloride (2.53 g, 36.4 mmol) were added and the resulting mixture was heated under reflux for 2 h. The ethanol was removed in vacuo and the crude product residue was purified by silica gel chromatography eluting with 10-100% ethyl acetate in hexane to give the title compound as a colourless oil (6.1 g, 87%).

Mass spectrum (API⁺): Found 351 (MNa⁺). C₁₉H₂₄N₂O₃ requires 328.

¹H NMR (CDCl₃) δ: 1.49 (9H, s), 2.35 (3H, s), 2.90-3.00 (4H, m), 3.50-3.65 (4H, m), 6.31 (1H, s), 7.21 (1H, d, J=8 Hz), 7.50-7.53 (2H, m).

### DESCRIPTION 30

### 7-(5-(3-Methyl)isoxazolyl)-2,3,4,5-tetrahydro-1H-3-benzazepine

A solution of 3-(tert-butyloxycarbonyl)-7-(5-(3-methyl)isoxazolyl)-2,3,4,5-tetrahydro-1H-3-benzazepine (5.1 g, 15.6 mmol) in CH₂Cl₂ (30 ml) and trifluoroacetic acid (10 ml) was heated under reflux for 2 h. Solvent was evaporated in vacuo and the residue partitioned between diethyl ether (150 ml) and water (150 ml). The aqueous phase was saturated with potassium carbonate then extracted with CH₂Cl₂ (2×200 ml). The combined organic extracts were dried and evaporated in vacuo to give the title compound (3.15 g, 88%).

Mass spectrum (API⁺): Found 229 (MH⁺). C₁₄H₁₆N₂O requires 228.

¹H NMR (CDCl₃) δ: 1.80 (1H, br s), 2.34 (3H, s), 2.90-3.10 (8H, m), 6.30 (1H, s), 7.17 (1H, d, J=8 Hz), 7.40-7.55 (2H, m).

### DESCRIPTION 31

### trans-3-(2-(1-(4-N-tert-Butyloxycarbonyl)amino)cyclohexyl)ethyl-7-(5-(3-methyl)isoxazolyl)-2,3,4,5-tetrahydro-1H-3-benzazepine

Prepared from 7-(5-(3-methyl)isoxazolyl)-2,3,4,5-tetrahydro-1H-3-benzazepine in a manner similar to Description 2, in 92% yield.

Mass spectrum (API⁺): Found 454 (MH⁺). C₂₇H₃₉N₃O₃ requires 453.

¹H NMR (CDCl₃) δ: 1.00-1.10 (4H, m), 1.15-1.25 (1H, m), 1.44 (9H, s), 1.55-1.70 (2H, m), 1.70-1.85 (2H, m), 1.95-2.05 (2H, m), 2.34 (3H, s), 2.45-2.55 (2H, m), 2.55-2.70 (4H, m), 2.90-3.00 (4H, m), 3.35 (1H, m), 4.30-4.40 (1H, m), 6.30 (1H, s), 7.16 (1H, d, J=8 Hz), 7.45-7.55 (2H, m).

### DESCRIPTION 32

### trans-3-(2-(1-(4-Amino)cyclohexyl)ethyl-7-(5-(3-methyl)isoxazolyl)-2,3,4,5-tetrahydro-1H-3-benzazepine

Prepared from trans-3-(2-(1-(4-N-tert-butyloxycarbonyl)amino)cyclohexyl)ethyl-7-(5-(3-methyl)isoxazolyl)-2,3,4,5-tetrahydro-1H-3-benzazepine in a manner similar to Description 3 in 99% yield.

¹H NMR (CDCl₃) δ: 0.90-1.10 (4H, m), 1.15-1.25 (1H, m), 1.35-1.50 (4H, m), 1.70-1.80 (2H, m), 1.80-1.90 (2H, m), 2.34 (3H, s), 2.42-2.52 (2H, m), 2.55-2.72 (5H, m), 2.90-3.00 (4H, m), 6.30 (1H, s), 7.16 (1H, d, J=8 Hz), 7.45-7.55 (2H, m).

### DESCRIPTION 33

### 3-(tert-Butyloxycarbonyl)-7-methanesulphonyloxy-2,3,4,5-tetrahydro-1H-3-benzazepine

A solution of 3-(tert-butyloxycarbonyl)-7-hydroxy-2,3,4,5-tetrahydro-1H-3-benzazepine (3.0 g, 0.011 mol), methanesulphonylchloride (1.44 g, 0.013 mol), triethylamine (1.27 g, 0.013 mol) and dichloromethane (50 ml) was stirred at room temperature for 18 h. The reaction mixture was then partitioned between dichloromethane (50 ml) and a saturated solution of sodium hydrogen carbonate (50 ml). The organic layer was separated, washed with water (50 ml) and then dried (Na₂SO₄). The solvent was then evaporated in vacuo to give the title compound (3.85 g, 99%) as a pale yellow oil.

¹H NMR (CDCl₃) δ: 1.48 (9H, s), 2.86-2.92 (4H, m), 3.13 (3H, s), 3.53-3.56 (4H, m), 7.00-7.03 (2H, m), 7.13-7.16 (1H, m).

Mass spectrum (AP⁺): Found 242 [M-BOC]H⁺. C₁₆H₂₃NSO₅ requires 341.

### DESCRIPTION 34

### 7-Methanesulphonyloxy-2,3,4,5-tetrahydro-1H-3-benzazepine

A solution of 3-(tert-butyloxycarbonyl)-7-methanesulphonyloxy-2,3,4,5-tetrahydro-1H-3-benzazepine (3.8 g, 0.011 mol), trifluoroacetic acid (3.76 g, 0.033 mol) and dichloromethane (50 ml) was heated at 50° C. for 5 h. The solvents were then evaporated in vacuo and the residue partitioned between water (200 ml) and ethyl acetate (150 ml). The aqueous layer was removed and washed with ethyl acetate (100 ml) and then basified to pH 14 with 40% sodium hydroxide. The suspension was then extracted with ethyl acetate (3×150 ml) and the combined organic layers dried (Na₂SO₄). The solvents were evaporated in vacuo to give the title compound (2.15 g, 80%) as a colourless oil.

¹H NMR (CDCl₃) δ: 2.88-3.00 (8H, m), 3.13 (3H, s), 6.99-7.03 (2H, m), 7.12 (1H, d).

Mass spectrum (AP⁺): Found 242 (MH)⁺. C₁₁H₁₅NSO₃ requires 241.

### DESCRIPTION 35

### trans-3-(2-(1-(4-N-tert-Butyloxycarbonyl)amino)cyclohexyl)ethyl-7-methanesulphonyloxy-2,3,4,5-tetrahydro-1H-3-benzazepine

A mixture of 7-(methanesulphonyloxy)-2,3,4,5-tetrahydro-1H-3-benzazepine (2.0 g, 8.3 mmol) and trans-2-(1-(4-N-tert-butyloxycarbonyl)amino)cyclohexyl acetaldehyde (1.37 g, 5.7 mmol) in dichloroethane (30 ml) was stirred at room temperature for 5 min, before sodium triacetoxyborohydride (1.69 g, 7.98 mmol) was added in a single portion. After stirring at room temperature for 48 h, a saturated solution of sodium hydrogen carbonate (50 ml) was added and the two layers separated. The aqueous layer was extracted with dichloromethane (3×60 ml) and the combined organic layers were dried (Na₂SO₄). The solvent was then evaporated in vacuo and the residue purified by column chromatography (silica gel, ethyl acetate) to give the title compound (2.54 g, 95%) as a white solid.

¹H NMR (CDCl₃) δ: 0.9-1.25 (7H, m), 1.44 (9H, s), 1.70-1.80 (2H, m), 1.90-2.05 (2H, m), 2.42-2.50 (2H, m), 2.55-2.65 (4H, m), 2.88-2.95 (4H, m), 3.12 (3H, s), 3.36 (1H, br s), 4.34 (1H, br s), 6.98-7.02 (2H, m), 7.08-7.12 (1H, d).

Mass spectrum (AP⁺): Found 467 [MH⁺]. C₂₄H₃₈N₂SO₅ requires 466.

### DESCRIPTION 36

### trans-3-(2-(1-(4-amino)cyclohexyl)ethyl)-7-methanesulphonyloxy-2,3,4,5-tetrahydro-1H-3-benzazepine

A solution of trans-3-(2-(1-(4-N-tert-butyloxycarbonyl)amino)cyclohexyl)ethyl-7-methanesulphonyloxy-2,3,4,5-tetrahydro-1H-3-benzazepine, trifluoroacetic acid (8 ml) and dichloromethane (32 ml), were stirred at room temperature for 2 h, under argon. The solvents were then evaporated in vacuo and the residue partitioned between water (150 ml) and ethyl acetate (60 ml). The aqueous layer was removed and washed with ethyl acetate (50 ml). The aqueous layer was then basified to pH 14 with 40% sodium hydroxide. The suspension was then extracted with ethyl acetate (3×80 ml) and the combined organic layers dried (Na₂SO₄). The solvents were evaporated in vacuo to give the title compound (1.78 g, 93%) as an oil which crystallised on standing.

¹H NMR (CDCl₃) δ: 0.95-1.45 (7H, m), 1.70-1.80 (2H, m), 1.80-1.90 (2H, m), 2.49 (2H, t, J=7.8 Hz), 2.55-2.65 (5H, m), 2.88-2.95 (4H, m), 3.12 (3H, s), 6.99-7.02 (2H, m), 7.11 (1H, d, J=8 Hz).

Mass Spectrum (AP⁺): Found 367 (MH⁺). C₁₉H₃₀N₂SO₃ requires 366.

### EXAMPLES

The Compounds of Examples tabulated below (Tables 1-3) were all prepared using the following general method:

A mixture of the appropriate trans-2-(2-(1-(4-amino)cyclohexyl)ethyl)-2,3,4,5-tetrahydro-1H-3-benzazepine (0.35 mmol), the appropriate acid (0.35 mmol), 1-ethyl-3-(3-dimethylaminopropyl)carbodiimide hydrochloride (0.35 mmol), 1-hydroxybenzotriazole (catalytic amount) and dichloromethane (5 ml) was shaken for 16 h. Saturated sodium bicarbonate (4 ml) was then added and the mixture shaken for 0.25 h. Chromatography of the organic layer on silica with 50-100% ethyl acetate in hexane and 0-10% methanol in ethyl acetate gradient elution gave the title compounds.

TABLE 1

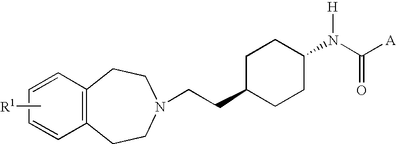

| | | | Characterising Data |
|---|---|---|---|
| Example | R¹ | A | Mass Spectrum(API⁺); ¹H NMR(CDCl₃) |
| 1 | H | 4-Quinolinyl | Found: 428(MH⁺). C₂₈H₃₃N₃O requires |
| | | | 427. |
| | | | δ: 1.06-1.37(5H, m), 1.47(2H, m), 1.78 |
| | | | (2H, m), 2.19(2H, m), 2.51(2H, m), 2.64 |
| | | | (4H, m), 2.93(4H, m), 4.03(1H, m), 5.90 |
| | | | (1H, d, J=8 Hz), 7.10(4H, m), 7.40(1H, d, |
| | | | J=4 Hz), 7.60(1H, m), 7.75(1H, m), 8.15 |
| | | | (2H, m), 8.91(1H, d, J=4 Hz). |
| 2 | H | trans- | Found: 481(MH⁺). C₂₈H₃₆N₂O₃S |
| | | CH═CHC₆H₄(3- | requires 480. |
| | | SO₂Me) | δ: 1.01-1.33(5H, m), 1.45(2H, m), 1.81 |
| | | | (2H, m), 2.02(2H, m), 2.50(2H, m), 2.63 |
| | | | (4H, m), 2.93(4H, m), 3.07(3H, s), 3.84 |
| | | | (1H, m), 5.64(1H, d, J=8 Hz), 6.49(1H, d, |
| | | | J=16 Hz), 7.09(4H, m), 7.64(3H, m), 7.89 |
| | | | (1H, d, J=8 Hz), 8.09(1H, s). |
| 3 | H | trans- | Found: 421(MH⁺). C₂₇H₃₃FN₂O requires |
| | | CH═CHC₆H₄(4-F) | 420. |
| | | | δ: 1.00-1.33(5H, m), 1.43(2H, m), 1.79 |
| | | | (2H, m), 2.04(2H, m), 2.49(2H, m), 2.62 |
| | | | (4H, m), 2.93(4H, m), 3.86(1H, m), 5.51 |
| | | | (1H, d, J=8 Hz), 6.26(1H, d, J=16 Hz), |
| | | | 7.11(6H, m), 7.47(2H, m), 7.56(1H, d, |
| | | | J=16 Hz). |
| 4 | H | 2-Indolyl | Found: 416(MH⁺). C₂₇H₃₃N₃O requires |
| | | | 415. |
| | | | δ: 1.05-1.37(5H, m), 1.46(2H, m), 1.82 |
| | | | (2H, m), 2.12(2H, m), 2.52(2H, m), 2.63 |
| | | | (4H, m), 2.92(4H, m), 3.96(1H, m), 5.98 |
| | | | (1H, d, J=8 Hz), 6.79(1H, m), 7.14(5H, |
| | | | m), 7.26(1H, m), 7.41(1H, m), 7.64(1H, d, |
| | | | J=8 Hz), 9.35(1H, br s). |
| 5 | H | —C₆H₄(3-(3- | Found: 454(MH⁺). C₃₀H₃₅N₃O requires |
| | | pyridyl)) | 453. |
| | | | δ: 1.08-1.37(5H, m), 1.46(2H, m), 1.84 |
| | | | (2H, m), 2.12(2H, m), 2.51(2H, m), 2.63 |
| | | | (4H, m), 2.92(4H, m), 3.95(1H, m), 6.02 |
| | | | (1H, d, J=8 Hz), 7.11(4H, m), 7.38(1H, |
| | | | m), 7.54(1H, t, J=8 Hz), 7.72(2H, m), |
| | | | 7.91(1H, m), 7.98(1H, s), 8.62(1H, m), |
| | | | 8.86(1H, d, J=2 Hz). |
| 6 | H | —CH₂Ph | Found 391(MH⁺). C₂₆H₃₄N₂O requires |
| | | | 390. |
| | | | δ: 0.87-1.14(5H, m), 1.40(2H, m), 1.68 |
| | | | (2H, m), 1.89(2H, m), 2.45(2H, m), 2.49 |
| | | | (4H, m), 2.89(4H, m), 3.54(2H, s), 3.68 |
| | | | (1H, m), 5.14(1H, m), 7.10(4H, m), 7.30 |
| | | | (5H, m). |
| 7 | H | —CH₂(3-indolyl) | Found: 430(MH⁺). C₂₈H₃₅N₃O requires |
| | | | 429. |
| | | | δ: 0.77-1.13(5H, m), 1.36(2H, m), 1.64 |
| | | | (2H, m), 1.84(2H, m), 2.42(2H, m), 2.57 |
| | | | (4H, m), 2.98(4H, m), 3.70(3H, m), 5.46 |
| | | | (1H, d, J=8 Hz), 7.12(7H, m), 7.39(1H, d, |
| | | | J=8 Hz), 7.53(1H, d, J=8 Hz), 8.24(1H, |
| | | | br s). |
| 8 | H | —CH₂(4-quinolyl) | Found: 442(MH⁺) C₂₉H₃₅N₃O requires |
| | | | 441. |
| | | | δ: 0.80-1.15(5H, m), 1.35-1.45(2H, m), |
| | | | 1.65-1.75(2H, m), 1.80-1.90(2H, m), |
| | | | 2.45-2.52(2H, m), 2.60-2.70(4H, m), |
| | | | 2.85-3.00(4H, m), 3.70(1H, m), 3.99(2H, |
| | | | s), 5.13(1H, d, J=8 Hz), 7.00-7.16(4H, |
| | | | m), 7.33(1H, d, J=3 Hz), 7.60(1H, m), |
| | | | 7.75(1H, m), 7.98(1H, d, J=8 Hz), 8.14 |
| | | | (1H, d, J=8 Hz), 8.88(1H, d, J=3 Hz). |
| 9 | 6-OMe | trans- | Found: 451(MH⁺), C₂₈H₃₅FN₂O₂ |
| | | CH═CHC₆H₄(4- | requires 450. |
| | | F) | δ: 1.10-1.30(5H, m), 1.40-1.50(2H, m), |
| | | | 1.75-1.85(2H, m), 2.00-2.10(2H, m), |
| | | | 2.40-2.52(2H, m), 2.55-2.70(4H, m), |
| | | | 2.85-3.10(4H, m), 3.80(3H, s), 3.86(1H, |
| | | | m), 5.37(1H, d, J=8 Hz), 6.26(1H, d, |
| | | | J=15 Hz), 6.74(2H, m), 7.06(3H, m), |
| | | | 7.47(2H, dd, J=9 Hz, 6 Hz), 7.54(1H, d, |
| | | | J=15 Hz). |
| 10 | 6-OMe | 4-Quinolyl | Found: 458(MH⁺), C₂₉H₃₅N₃O₂ requires |
| | | | 457 |
| | | | δ: 1.05-1.35(5H, m), 1.55-1.69(2H, m), |
| | | | 1.70-1.80(2H, m), 2.10-2.24(2H, m), |
| | | | 2.90-3.35(10H, m), 3.80(3H, s), 4.00 |
| | | | (1H, m), 5.85(1H, d, J=8 Hz), 6.75(2H, |
| | | | m), 7.13(1H, t, J=8 Hz), 7.41(1H, d, |
| | | | J=4 Hz), 7.60(1H, m), 7.78(1H, m), |
| | | | 8.16(2H, m), 8.94(1H, d, J=4 Hz). |
| 11 | 6-OMe | 3-(pyrrolo[2,3- | Found: 447(MH⁺), C₂₇H₃₄N₄O₂ requires |
| | | b]pyridyl) | 446. |
| | | | δ: 1.30-1.35(5H, m), 1.45-1.55(2H, m), |
| | | | 1.75-1.80(2H, m), 2.10-2.20(2H, m), |
| | | | 2.55-2.85(6H, m), 2.90-3.20(4H, m), |
| | | | 3.80(3H, s), 3.96(1H, m), 5.65(1H, d, |
| | | | J=8 Hz), 6.74(2H, m), 7.10(1H, t, |
| | | | J=8 Hz), 7.20(1H, m), 7.80(1H, s), |
| | | | 8.35(2H, m), 9.45(1H, br s). |
| 30 | 7-CN | —CH₂Ph(2,5-diF) | Found: 452(MH⁺); C₂₇H₃₁F₂N₃O |
| | | | requires 451. |
| | | | δ: 0.80-1.00(4H, m), 1.10(1H, m), 1.40-1.50 |
| | | | (2H, m), 1.55-1.65(2H, m), 1.80-1.90 |
| | | | (2H, m), 2.70-2.80(2H, m), 2.90-3.20 |
| | | | (8H, m), 3.48(2H, s), 3.60(1H, m), |
| | | | 5.28(1H, d, J=8 Hz), 6.90-7.05(3H, m), |
| | | | 7.21(1H, d, J=8 Hz), 7.40(1H, s), 7.45 |
| | | | (1H, d, J=6 Hz). |
| 31 | 7-CN | —CH₂(2-naphthyl) | Found: 466(MH⁺); C₃₁H₃₅N₃O requires |
| | | | 465. |
| | | | δ: 0.80-1.20(5H, m), 1.30-1.40(2H, m), |
| | | | 1.65-1.75(2H, m), 1.85-1.90(2H, m), |
| | | | 2.35-2.50(2H, m), 2.50-2.60(4H, m), |
| | | | 2.85-3.00(4H, m), 3.60-3.85(3H, m), |
| | | | 5.16(1H, d, J=9 hz), 7.10(1H, d, J=9 Hz), |
| | | | 7.30-7.40(3H, m), 7.40-7.60(2H, |
| | | | m), 7.70(1H, s), 7.80-7.90(3H, m). |
| 32 | 7-CN | trans- | Found: 464(MH⁺); C₂₈H₃₁N₃OF₂ |
| | | CH═CHC₆H₃(2,4- | requires 463. |
| | | diF) | δ: 1.02-1.30(5H, m), 1.40-1.48(2H, m), |
| | | | 1.78-1.82(2H, m), 2.03-2.06(2H, m), |
| | | | 2.50(2H, t, J=8 Hz), 2.62-2.66(4H, m), |
| | | | 2.93-3.00(4H, m), 3.79-3.91(1H, m), |
| | | | 5.72(1H, d, J=8 Hz), 6.43(1H, d, J=16 Hz), |
| | | | 6.75-6.95(2H, m), 7.18(1H, d, J=8 Hz), |
| | | | 7.38-7.47(3H, m), 7.59(1H, d, |
| | | | J=16 Hz). |
| 33 | 7-CN | trans- | Found: 464(MH⁺); C₂₈H₃₁N₃OF₂ |
| | | CH═CHC₆H₃(2,5- | requires 463. |
| | | diF) | (CD₃OD) δ: 1.06-1.40(5H, m), 1.60-1.74 |
| | | | (2H, m), 1.80-1.85(2H, m), 1.92-1.97 |
| | | | (2H, m), 2.90-3.40(8H, m), 3.64-3.79 |
| | | | (3H, m), 6.64(1H, d, J=16 Hz), 7.09-7.18 |
| | | | (2H, m), 7.29-7.39(2H, m), 7.52-7.58 |
| | | | (3H, m). |
| 34 | 7-CN | trans- | Found: 446(MH⁺); C₂₈H₃₂N₃OF requires |
| | | CH═CH₆H₄(3- | 445. |
| | | F) | δ: 1.03-1.35(5H, m), 1.40-1.48(2H, m), |
| | | | 1.75-2.10(4H, m), 2.51(2H, t, J=8 Hz), |
| | | | 2.61-2.65(4H, m), 2.93-2.99(4H, m), |
| | | | 3.72-3.88(1H, m), 6.36(1H, d, J=18 Hz), |
| | | | 7.04(1H, m), 7.17-7.45(7H, m), 7.55(1H, |
| | | | d, J=16 Hz) |
| 35 | | —CH₂CH₂C₆H₅ | Found: 430(MH⁺); C₂₈H₃₅N₃O requires |
| | | | 429. |
| | | | δ: 0.90-1.25(5H, m), 1.30-1.45(2H, m), |
| | | | 1.65-1.95(4H, m), 2.35-2.50(4H, m), |
| | | | 2.55-2.65(4H, m), 2.90-3.04(6H, m), |
| | | | 3.66-3.69(1H,m ), 5.09(1H, d, J=8 Hz), |
| | | | 7.15-7.35(6H, m), 7.37-7.42(2H, m). |
| 36 | 7-CN | trans- | Found: 446(MH⁺), C₂₈H₃₂FN₃O requires |
| | | CH═CHC₆H₄(2- | 445. |
| | | F) | δ: 1.00-1.30(5H, m), 1.40-1.50(2H, m), |
| | | | 1.75-1.85(2H, m), 2.00-2.10(2H, m), |
| | | | 2.45-2.50(2H, m), 2.60-2.70(4H, m), |
| | | | 2.85-3.00(4H, m), 3.80-3.95(1H, m), |
| | | | 5.43(1H, d, J=8 Hz), 6.47(1H, d, J=16 Hz), |
| | | | 7.00-7.20(3H, m), 7.26-7.35(1H, |
| | | | m), 7.30-7.55(3H, m), 7.66(1H, d, J=16 Hz). |
| 37 | 7-CN | 8-(1,4-dihydro-4- | Found: 469(MH⁺); C₂₉H₃₂N₄O₂ requires |
| | | oxo)-quinolinyl | 468. |
| | | | δ: 1.05-1.35(5H, m), 1.40-1.50(2H, m), |
| | | | 1.80-1.90(2H, m), 2.10-2.20(2H, m), |
| | | | 2.45-2.55(2H, m), 2.60-2.70(4H, m), |
| | | | 2.90-3.00(4H, m), 3.95(1H, m), 6.30-6.40 |
| | | | (2H, m), 7.18(1H, d, J=8 Hz), 7.32 |
| | | | (1H, t, J=8 Hz), 7.37(1H, s), 7.41(1H, d, |
| | | | J=8 Hz), 7.67(1H, t, J=5 Hz), 7.80(1H, d, |
| | | | J=7 Hz), 8.52(1H, d, J=8 Hz), 12.50(1H, |
| | | | br s). |
| 38 | 7-CN | 2-naphthyl | Found: 452(MH⁺); C₃₀H₃₃N₃O requires |
| | | | 451. |
| | | | δ: 1.09-1.26(5H, m), 1.42-1.50(2H, m), |
| | | | 1.80-1.87(2H, m), 2.10-2.25(2H, m), |
| | | | 2.40-2.54(2H, m), 2.61-2.70(4H, m), |
| | | | 2.92-2.99(4H, m), 3.95-4.00(1H, m), |
| | | | 5.81(1H, d, J=8 Hz), 7.18(1h, d, J=8 Hz), |
| | | | 7.30-7.59(6H, m), 7.84-7.95(2H, |
| | | | m), 8.20-8.29(1H, m). |
| 39 | 7-CN | trans- | Found: 458(MH⁺); C₂₉H₃₅N₃O₂ requires: |
| | | CH═CHC₆H₄(2- | 457 |
| | | OMe) | (DMSO-d₆) δ: 0.99-1.07(2H, m), 1.15-1.28 |
| | | | (3H, m), 1.35-1.40(2H, m), 1.78-1.88 |
| | | | (4H, m), 2.46(2H, t, J=7 Hz), 2.58 |
| | | | (4H, m, obscurred by DMSO), 2.90-2.96 |
| | | | (4H, m), 3.58-2.64(1H, m), 3.87(3H, s), |
| | | | 6.64(1H, d, J=16 Hz), 6.99(1H, t, J=7 Hz), |
| | | | 7.09(1H, d, J=8 Hz), 7.34-7.39(2H, |
| | | | m), 7.49-7.51(1H, m), 7.58-7.66(3H, m), |
| | | | 7.93-7.96(1H, m). |
| 40 | 7-CN | trans- | Found: 458(MH⁺); C₂₉H₃₅N₃O₂ requires |
| | | CH═CHC₆H₄(3- | 457. |
| | | OMe) | (DMSO-d₆) δ: 0.80-0.98(2H, m), 1.00-1.20 |
| | | | (3H, m), 1.20-1.35(2H, m), 1.78-1.82 |
| | | | (2H, m), 1.88-1.92(2H, m), 2.49(2H, |
| | | | t, J=8 Hz), 2.51-2.60(4H, m, obscurred |
| | | | by DMSO), 2.90-3.00(4H, m), 3.62(1H, |
| | | | m), 3.83(3H, s), 6.65(1H, d, J=16 Hz), |
| | | | 7.00(1H, m), 7.15-7.17(2H, m), 7.35-7.43 |
| | | | (3H, m), 7.61-7.63(2H, m), 7.99(1H, |
| | | | d, J=8 Hz). |
| 41 | 7-CN | trans- | Found: 458(MH⁺); C₂₉H₃₅N₃O₂ requires: |
| | | CH═CHC₆H₄(4- | 457. |
| | | OMe) | (DMSO-d₆) δ: 1.00-1.10(2H, m), 1.15-1.27 |
| | | | (3H, m), 1.38-1.41(2H, m), 1.77-1.80 |
| | | | (2H, m), 1.85-1.87(2H, m), 2.47(2H, |
| | | | t, J=7 Hz), 2.53-2.57(4H, m, obscurred |
| | | | by DMSO), 2.91-2.97(4H, m), 3.58-3.65 |
| | | | (1H, m), 3.81(3H, s), 6.48(1H, d, J=16 Hz), |
| | | | 7.00(2H, m), 7.35-7.40(2H, m), 7.51 |
| | | | (2H, m), 7.58-7.52(2H, m), 7.88(1H, d, |
| | | | J=8 Hz). |
| 42 | 7-CN | trans- | Found: 470(MH⁺); C₃₀H₃₅N₃O₂ requires |
| | | CH═CHC₆H₄(2- | 469. |
| | | COMe) | (DMSO-d₆) δ: 0.90-1.40(7H, m), 1.60-2.90 |
| | | | (4H, m), 2.46(2H, t, J=8 Hz), 2.46-2.54 |
| | | | (4H, m, obscurred by DMSO), 2.52 |
| | | | (3H, s), 2.80-3.00(4H, m), 3.60(1H, m), |
| | | | 6.47(1H, d, J=16 Hz), 7.35(2H, d, J=8 Hz), |
| | | | 7.40-7.63(4H, m), 7.75(1H, d, J=16 Hz), |
| | | | 7.89(1H, d, J=8 Hz), 8.04(1H, d, |
| | | | J=8 Hz). |
| 43 | 7-CN | trans- | Found: 470(MH⁺); C₃₀H₃₅N₃O₂ requires: |
| | | CH═CHC₆H₄(4- | 469. |
| | | COMe) | (DMSO-d₆): 0.90-1.40(7H, m), 1.73-1.88 |
| | | | (4H, m), 2.43(2H, t, J=8 Hz), 2.47-2.54 |
| | | | (4H, m, obscurred by DMSO), 2.59(3H, s), |
| | | | 2.80-2.93(4H, m), 3.50-3.70(1H, m), |
| | | | 6.73(1H, d, J=16 Hz), 7.32(1H, d, J=7hz), |
| | | | 7.45(1H, d, J=16 Hz), 7.55-7.59 |
| | | | (2H, m), 7.68(2H, d, J=8 Hz), 7.98(2H, |
| | | | m), 8.08(1H, d, J=8 Hz). |
| 44 | 7-CN | trans- | Found: 453(MH⁺); C₂₉H₃₂N₄O requires |
| | | CH═CHC₆H₄H4(2- | 452. |
| | | CN) | (DMSO-d₆) δ: 1.02-1.09(2H, m), 1.10-1.35 |
| | | | (3H, m), 1.36-1.42(2H, m), 1.78-1.81 |
| | | | (2H, m), 1.88-1.90(2H, m), 2.47(2H, |
| | | | t, J=7 Hz), 2.50-2.59(4H, m, obscurred |
| | | | by DMSO), 2.92-2.97(4H, m), 3.62-3.58 |
| | | | (1H, m), 6.85(1H, d, J=16 Hz), 7.36(1H, |
| | | | d, J=8 Hz), 7.58-7.67(4H, m), 7.79-7.89 |
| | | | (2H, m), 7.92-7.95(1H, m), 8.22-8.25 |
| | | | 1H, m). |
| 45 | 7-CN | trans | Found 453(MH⁺); C₂₉H₃₂N₄O requires: |
| | | CH═CHC₆H₄(3- | 452. |
| | | CN) | (DMSO-d₆) δ: 0.94-1.38(7H, m), 1.70-1.87 |
| | | | (4H, m), 2.43(2H, t, J=7 Hz), 2.46-2.59 |
| | | | (4H, m, obscurred by DMSO), 2.85-2.97 |
| | | | (4H, m), 3.52-3.65(1H, m), 6.72(1H, |
| | | | d, J=16 Hz), 7.32(1H, d, J=8 Hz), 7.42 |
| | | | (1H, d, J=16 Hz), 7.55-7.62(3H, m), 7.80-7.91 |
| | | | (2H, m), 8.02(1H, s), 8.09(1H, d, |
| | | | J=8 Hz). |
| 46 | 7-CN | —C₆H₄(3-(5-(3- | Found 483(MH⁺); C₃₀H₃₄N₄O₂ requires |
| | | methyl)isoxazolyl) | 482. |
| | | | (DMSO-d₆) δ: 0.96-1.10(2H, m), 1.23-1.50 |
| | | | (5H, m), 1.70-1.89(4H, m), 2.31(3H, |
| | | | s), 2.42-2.55(6H, m, obscurred by |
| | | | DMSO), 2.80-2.95(4H, m), 3.75(1H, m), |
| | | | 6.96(1H, s), 7.33(1H, d, J=8 Hz), 7.50-7.60 |
| | | | (3H, m), 7.90-8.00(2H, m), 8.28(1H, |
| | | | m), 8.41(1H, d, J=8 Hz). |
| 47 | 7-CN | 7-(1,2-dihydro-2- | Found 469(MH⁺); C₂₉H₃₂N₄O₂ requires |
| | | oxo)quinolinyl | 468. |
| | | | (DMSO-TFA) δ: 0.98-1.45(5H, m), 1.56-1.63 |
| | | | (1.56-1.63(2H, m), 1.75-1.89(4H, |
| | | | m), 2.95-3.32(8H, m), 3.65-3.85(3H, m), |
| | | | 5.67(1H, s), 6.60(1H, d, J=10 Hz), 7.41 |
| | | | (1H, d, J=8 Hz), 7.59-7.70(4H, m), 7.75 |
| | | | (1H, s), 7.90(1H, d, J=10 Hz), 8.32(1H, |
| | | | d), 9.69(1H, s). |
| 48 | 7-CN | cis-CH═CHC₆H₅ | Found 428(MH⁺); C₂₈H₃₃N₃O requires |
| | | | 427. |
| | | | δ: 0.80-1.15(5H, m), 1.30-1.40(2H, m), |
| | | | 1.65-1.75(2H, m), 1.80-1.95(2H, m), |
| | | | 2.40-2.50(2H, m), 2.55-2.65(4H, m), |
| | | | 2.85-3.00(4H, m), 3.75(1H, m), 5.25(1H, |
| | | | d, J=8 Hz), 5.98(1H, d, J=12.5 Hz), 6.76 |
| | | | (1H, d, J=12.5 Hz), 7.17(1H, d, J=8 Hz), |
| | | | 7.30-7.45(7H, m). |
| 49 | 7-CN | trans-CH═CH(2- | Found: 429(MH⁺); C₂₇H₃₂N₄O requires |
| | | pyridyl) | 428. |
| | | | δ (DMSO + TFA): 0.90-1.30(5H, m), 1.55-1.70 |
| | | | (2H, m), 1.70-1.80(2H, m), 1.80-1.90 |
| | | | (2H, m), 2.90-3.30(8H, m), 3.50-3.80 |
| | | | (3H, m), 7.08(1H, d, J=16 Hz), 7.40-7.50 |
| | | | (2H, m), 7.55-7.60(1H, m), 7.65-7.80 |
| | | | (3H, m), 8.05(1H, m), 8.25(1H, d, |
| | | | J=8 Hz), 8.70(1H, m), 9.70(1H, br s). |
| 50 | 7-CN | trans-CH═CH(1- | Found: 496(MH⁺); C₃₂H₃₄FN₃O requires |
| | | (4- | 495. |
| | | fluoro)naphthyl) | δ: (DMSOd₆ + TFA); 0.97-1.41(5H, m), |
| | | | 1.63(2H, m), 1.79(2H, m), 1.90(2H, m), |
| | | | 3.06(2H, m), 3.23(6H, m), 3.70(3H, m), |
| | | | 6.64(1H, d, J=16 Hz), 7.47(2H, m), 7.73 |
| | | | (5H, m), 8.12(3H, m), 8.24(1H, m). |
| 51 | 7-CN | trans-CH═CH(6- | Found: 486(MH⁺); C₃₀H₃₅N₃O requires |
| | | benzodioxanyl) | 485. |
| | | | δ: (DMSO d₆ + TFA): 0.93-1.33(5H, m), |
| | | | 1.60(2H, m), 1.81(4H, m), 3.04(2H, m), |
| | | | 3.17(6H, m), 3.67(3H, m), 4.26(4H, s), |
| | | | 6.42(1H, d, J=16 Hz), 6.87(1H, d, |
| | | | J=9 Hz), 7.03(2H, m), 7.27(1H, d, J=16 Hz), |
| | | | 7.46(1H, d, J=8 Hz), 7.73(2H, m), 7.90 |
| | | | (1H, d, J=8 Hz), 9.78(1H, br s). |
| 52 | 7-CN | trans-CH═CH(3- | Found: 485(MNa⁺); C₃₀H₃₃FN₄O |
| | | indolyl[5-F]) | requires 484. |
| | | | (DMSO-d₆) δ: 0.98-1.08(2H, m), 1.11-1.28 |
| | | | (3H, m), 1.35-1.42(2H, m), 1.75-1.80 |
| | | | (2H, m), 1.87-1.91(2H, m), 2.47(2H, |
| | | | t, J=7 Hz), 2.52-2.59(4H, m, obscurred |
| | | | by DMSO), 2.89-2.94(4H, m), 3.55-3.62 |
| | | | (1H, m), 6.56(1H, d, J=16 Hz), 7.03-7.09 |
| | | | (1H, m), 7.34(1H, d, J=8 Hz), 7.44-7.49 |
| | | | (1H, m), 7.56-7.75(5H, m), 7.80(1H, s), |
| | | | 11.63(1H, s). |
| 53 | 7-CN | trans-CH═CH(6- | Found: 482(MH⁺); C₃₀H₃₅N₅O requires |
| | | benzimidazolyl[1- | 481. |
| | | methyl] | (DMSO-d₆) δ: 0.98-1.07(2H, m), 1.16-1.27 |
| | | | (3H, m), 1.30-1.40(2H, m), 1.75-1.79 |
| | | | (2H, m), 1.84-1.89(2H, m), 2.46(2H, |
| | | | t, J=7 Hz), 2.50-2.55(4H, m, obscurred |
| | | | by DMSO), 2.90-2.97(4H, m), 3.60-3.66 |
| | | | (1H, m), 3.87(3H, s), 6.64(1H, d, J=16 HZ), |
| | | | 7.34(1H, d, J=8 Hz), 7.41-7.45(1H, |
| | | | m), 7.53-7.45(1H, m), 7.53-7.61(3H, m), |
| | | | 7.66(1H, d, J=8 Hz), 7.77(1H, s), |
| | | | 7.91-7.94(1H, m), 8.24(1H, s). |
| 54 | 7-CN | trans-CH═CH(7- | Found: 468(MH⁺); C₃₀H₃₃N₃O₂ requires |
| | | benzofuranyl) | 467. |
| | | | (DMSO/TFA) δ: 1.02-1.43(5H, m), 1.65-1.75 |
| | | | (2H, m), 1.75-2.00(4H, m), 3.08-3.35 |
| | | | (8H, m), 3.65-3.80(3H, m), 7.09-7.15 |
| | | | (2H, m), 7.36(1H, t); 7.48-7.56(2H, |
| | | | m), 7.66(1H, d, J=15.8 Hz), 7.73-7.80 |
| | | | (3H, m), 8.15(1H, d, J=2.2 Hz), 8.25(1H, |
| | | | d). |
| 55 | 7-CN | trans-CH═CH(5- | Found: 481(MH⁺) C₃₁H₃₆N₄O requires |
| | | indolyl[3-methyl]) | 480. |
| | | | (DMSO/TFA) δ: 0.95-1.35(5H, m), 1.55-1.70 |
| | | | (2H, m), 1.70-1.95(4H, m), 2.27(3H, |
| | | | s), 2.95-3.30(8H, m), 3.55-3.80(3H, m), |
| | | | 6.52(1H, d, J=15.7 Hz), 7.14(1H, s), 7.33 |
| | | | (2H, m), 7.46(2H, m), 7.71(3H, m), 7.84 |
| | | | (1H, d), 9.82(1H, br s). |
| 56 | 7-CN | trans-CH═CH(6- | Found: 483(MH⁺); C₃₀H₃₄N₄O₂ requires |
| | | (2,3-dihydro-2- | 482. |
| | | oxo)indolyl) | (DMSO) δ: 0.87-1.36(7H, m), 1.74-1.90 |
| | | | (4H, m), 2.44(2H, t, J=7.2 Hz), 2.50-2.65 |
| | | | (4H, m, under DMSO), 2.80-2.95(4H, m), |
| | | | 3.49(2H, s), 3.57-3.70(1H, m), 6.54(1H, |
| | | | d, J=15.8 Hz), 6.95(1H, s), 7.09(1H, d, |
| | | | J=8), 7.22(1H, d, J=7.5), 7.27-7.39(2H, |
| | | | m), 7.56-7.60(2H, m), 7.92(1H, d). |
| 57 | 7-CN | —CH₂(2- | Found: 456(MH⁺); C₂₉H₃₃N₃O₂ requires |
| | | benzofuranyl) | 455. |
| | | | (DMSO) δ: 0.95-1.23(5H, m), 1.31-1.36 |
| | | | (2H, m), 1.72-1.82(4H, m), 2.42(2H, t, |
| | | | J=7.4 Hz), 2.49-2.53(4H, m, under DMSO), |
| | | | 2.88-2.93(4H, m), 3.47-3.52(1H, m), |
| | | | 3.63(2H, s), 6.65(1H, s), 7.19-7.24(2H, |
| | | | m), 7.31(1H, d, J=7.7 Hz), 7.49(1H, d, |
| | | | J=7.9 Hz), 7.55-7.59(3H, m), 8.02(1H, d). |
| 58 | 7-CN | trans-CH═CH(4- | Found: 479(MH⁻); C₃₁H₃₆NO₄ requires |
| | | indolyl[2-methyl]) | 480. |
| | | | (DMSO + TFA) δ: 0.83-1.25(5H, m), 1.48-1.55 |
| | | | (2H, m), 1.63-1.67(2H, m), 1.75-1.80 |
| | | | (2H, m), 2.31(3H, s), 2.85-3.20(8H, |
| | | | m), 3.45-3.65(3H, m), 6.36(1H, s), 6.62 |
| | | | (1H, d, J=16 Hz), 6.89(1H, t, J=8 Hz), |
| | | | 7.00(1H, d, J=7 Hz), 7.18(1H, d, J=8 Hz), |
| | | | 7.34(1H, d, J=8 Hz), 7.49(1H, d, J=16 Hz), |
| | | | 7.55-7.62(2H, m), 7.90(1H, d, J=8 Hz), |
| | | | 9.75(1H, b s), 11.09(1H, s). |
| 59 | 7-CN | trans-CH═CH(5- | Found: 466(MH⁻); C₂₉H₃₃N₅O requires |
| | | benzimidazolyl) | 467. |
| | | | (DMSO + TFA) δ: 1.02-1.35(5H, m), 1.57-1.61 |
| | | | (2H, m), 1.75-1.78(2H, m), 1.90-1.93 |
| | | | (2H, m), 3.00-3.30(8H, m), 3.65-3.70 |
| | | | (3H, m), 6.66 and 6.73(1H, 2 × d, J=16 Hz), |
| | | | 7.43(1H, d, J=8 Hz), 7.60-8.08 |
| | | | (6H, m), 8.00 and 8.06(1H, 2 × s), 9.59(!H, |
| | | | m), 9.88(1H, b s). |
| 60 | 7-CN | trans- | Found: 428(MH⁺); C₂₈H₃₃N₃O requires |
| | | CH═CHC₆H₅ | 427. |
| | | | (DMSO-d₆ + TFA) δ: 0.96-1.36(5H, m), |
| | | | 1.62(2H, m), 1.81(4H, m), 3.05(2H, m), |
| | | | 3.18(6H, m), 3.67(3H, m), 6.60(1H, d, J=16 Hz), |
| | | | 7.27-7.59(7H, m), 7.72(2H, m), |
| | | | 7.99(1H, d, J=8 Hz), 9.72(1H, br s). |
| 61 | 7-CN | trans- | Found: 472(MH⁺); C₂₉H₃₃N₃O₃ requires |
| | | CH═CHC₆H₃(2,3- | 471. |
| | | methylenedioxy) | (DMSO-d₆ + TFA) δ: 0.94-1.32(5H, m), |
| | | | 1.61(2H, m), 1.82(4H, m), 3.03(2H, m), |
| | | | 3.18(6H, m), 3.64(3H, m), 6.13(2H, s), |
| | | | 6.71(1H, d, J=16 Hz), 6.94(3H, m), 7.32 |
| | | | (1H, d, J=16 Hz), 7.46(1H, d, J=8 Hz), |
| | | | 7.71(2H, m), 8.09(1H, d, J=8 Hz), 9.75 |
| | | | (1H, brs). |
| 62 | 7-CN | trans- | Found: 511(MH⁺); C₃₂H₃₈N₄O₂ requires |
| | | CH═CHC₆H₄(3- | 510. |
| | | (1-(2- | (DMSO-d₆) δ: 0.9-1.28(5H, m), 1.35(2H, |
| | | oxo)pyrrolidinyl)) | m), 1.79(4H, m), 2.07(2H, m), 2.48(8H, |
| | | | m), 2.91(4H, m), 3.59(1H, m), 3.86(2H, t, |
| | | | J=7 Hz), 6.60(1H, d, J=16 Hz), 7.34(4H, |
| | | | m), 7.60(3H, m), 7.89(1H, m), 7.99(1H, d, |
| | | | J=8 Hz). |
| 63 | 7-CN | —CH₂(2-indolyl) | Found: 455(MH⁺); C₂₉H₃₄N₄O requires |
| | | | 454. |
| | | | (DMSO-d₆) δ: 0.96(2H, m), 1.15(3H, m), |
| | | | 1.34(2H, m), 1.76(4H, m), 2.42(2H, m), |
| | | | 2.50(4H, m), 2.90(4H, m), 3.45(1H, m), |
| | | | 3.53(2H, s), 6.17(1H, m), 6.94(2H, m), |
| | | | 7.32(2H, m), 7.41(1H, d, J=8 Hz), 7.56 |
| | | | (2H, m), 7.87(1H, d, J=8 Hz), 10.85(1H, |
| | | | br s). |
| 64 | 7-CN | —CH₂(2- | Found: 472(MH⁺); C₂₉H₃₃N₃SO requires |
| | | benzothiophenyl) | 471. |
| | | | (DMSO) δ: 0.95-1.20(5H, m), 1.31-1.35 |
| | | | (2H, m), 1.71-1.81(4H, m), 2.42(2H, t, J=7.4 Hz), |
| | | | 2.50-2.53(4H, m), 2.87-2.93 |
| | | | (4H, m), 3.44-3.48(1H, m), 3.70(2H, s), |
| | | | 7.19(1H, s), 7.27-7.32(3H, m), 7.55-7.58 |
| | | | (2H, m), 7.75(1H, d, J=7.4 Hz), 7.88 |
| | | | (1H, d, J=7.8 Hz), 8.04(1H, m). |
| 65 | | trans-CH═CH(2- | Found: 512 & 514(MH⁺); C₂₆H₃₀N₃SOBr |
| | | thiophenyl[3-Br]) | requires 511 & 513. |
| | | | (DMSO) δ: 0.95-1.40(7H, ), 1.72-1.85 |
| | | | (4H, m), 2.42(2H, m), 2.50-2.58(4H, m, |
| | | | under DMSO), 2.87-2.95(4H, m), 3.54-3.62 |
| | | | (1H, m), 6.48(1H, d), 7.19(1H, d, J=5.4 Hz), |
| | | | 7.32(1H, d), 7.48(1H, d), 7.55-7.60 |
| | | | (2H, m), 7.72(1H, d), 8.05(1H, d). |
| 66 | 7-CN | —C₆H₄(3-(2- | Found: 479(MH⁺); C₃₁H₃₄N₄O requires |
| | | pyridyl)) | 478. |
| | | | (DMSO-d₆) δ: 1.04-1.37(2H, m), 1.28-1.47 |
| | | | (5H, m), 1.81-1.97(4H, m), 2.49(2H, |
| | | | t, J=7 Hz), 2.56-2.61(4H, m, obscurred |
| | | | by DMSO), 2.92-3.00(4H, m), 3.79-3.88 |
| | | | (1H, m), 7.38(1H, d, J=8 Hz), 7.44-7.47 |
| | | | (1H, m), 7.60-7.66(3H, m), 7.90-8.00 |
| | | | (2H, m), 8.06-8.10(1H, m), 8.25-8.27 |
| | | | (1H, m), 8.37-8.40(1H, m), 8.55(1H, s), |
| | | | 8.73-8.76(1H, m). |
| 67 | 7-CN | —C₆H₄(3-(5- | Found: 480(MH⁺); C₃₀H₃₃N₅O requires |
| | | pyrimidinyl) | 479. |
| | | | (DMSO-d₆) & [HCl salt] δ: 1.05-1.12(2H, |
| | | | m), 1.30-1.41(3H, m), 1.65-1.70(2H, m), |
| | | | 1.78-1.82(2H, m), 1.88-1.92(2H, m), |
| | | | 2.96-3.04(2H, m), 3.08-3.20(4H, m), |
| | | | 3.30-3.45(2H, m), 3.65-3.71(2H, m), |
| | | | 3.75-3.80(1H, m), 7.45(1H, d, J=8 Hz), |
| | | | 7.60-7.73(2H, m), 7.92-7.98(3H, m), |
| | | | 8.23(1H, s), 8.32-8.36(1H, m), 9.21-9.23 |
| | | | (3H, m), 10.67(1H, s). |
| 68 | 7-CN | —C₆H₄(3-C₆H₄(4- | Found: 503(MH⁺); C₃₃H₃₄N₄O requires |
| | | CN)) | 502. |
| | | | (DMSO-d₆) δ: 1.00-1.11(2H, m), 1.20-1.43 |
| | | | (5H, m), 1.79-1.83(2H, m), 1.88-1.93 |
| | | | (2H, m), 2.48(2H, t, J=7 Hz), 2.52-2.58 |
| | | | (4H, m, obscurred by DMSO), 2.91-2.96 |
| | | | (4H, m), 3.75-8.83(1H, m), 7.34- |
| | | | (1H, d, J=8 Hz), 7.58-7.61(3H, m), 7.83-7.89 |
| | | | 7.89(3H, m), 7.95-8.05(3H, m), 8.20(1H, |
| | | | s), 8.33-8.35(1H, m). |
| 69 | 7-CN | —C₆H₄(3-(3-(5- | Found: 498(MH⁺); C₃₀H₃₅N₅O₂ requires |
| | | ethyl)-1,2,4- | 497. |
| | | oxadiazolyl) | δ: 1.07-1.33(5H, m), 1.41-1.50(5H, m), |
| | | | 1.82-1.86(2H, m), 2.09-2.13(2H, m), |
| | | | 2.48-2.54(2H, m), 2.61-2.64(4H, m), |
| | | | 2.90-3.10(6H, m), 3.89-4.04(1H, m), |
| | | | 6.05(1H, d, J=8 Hz), 7.18(1H, d, J=8 Hz), |
| | | | 7.38-7.43(2H, m), 7.57(1H, t, J=8 Hz), |
| | | | 7.99(1H, dd, J=8 Hz and 1 Hz), 8.20 |
| | | | (1H, dd, J=8 Hz and 1 Hz), 8.33(1H, d, |
| | | | J=1 Hz). |
| 70 | 7-CN | trans-CH═CH(2- | Found: 434(MH⁺); C₂₆H₃₁N₃OS requires |
| | | thiophenyl) | 433. |
| | | | (DMSOd₆) δ: 0.85-1.30(5H, m), 1.37(2H, |
| | | | m), 1.90(4H, m), 2.45-2.75(6H, m), 3.00 |
| | | | (4H, m), 3.58(1H, m), 6.35(1H, d, J=16 Hz), |
| | | | 7.10(1H, m), 7.35(2H, m), 7.45-7.65 |
| | | | (4H, m), 7.97(1H, d, J=16 Hz). |
| 71 | 7-CN | trans-CH═CH(2- | Found: 418(MH⁺); C₂₆H₃₁N₃O₂ requires |
| | | furanyl) | 417. |
| | | | (DMSOd₆) δ: 0.80-1.30(5H, m), 1.37(2H, |
| | | | m), 1.78(4H, m), 2.30-2.70(6H, m), 2.93 |
| | | | (4H, m), 3.55(1H, m), 6.38(1H, d, J=16 Hz), |
| | | | 6.55(1H, dd, J=3.2 Hz), 6.74(1H, d, |
| | | | J=3 Hz), 7.19(1H, d, J=16 Hz), 7.33(1H, |
| | | | d, J=8 Hz), 7.57(2H, m), 7.75(1H, s), |
| | | | 8.00(1H, d, J=8 Hz). |
| 72 | 7-CN | trans-CH═CH(3- | Found: 434(MH⁺); C₂₆H₃₁N₃OS requires |
| | | thiophenyl) | 433. |
| | | | (DMSOd₆) δ: 0.85-1.30(5H, m), 1.40(2H, |
| | | | m), 1.80(4H, m), 2.35-2.70(6H, m), 2.90 |
| | | | (4H, m), 3.60(1H, m), 6.40(1H, d, J=16 Hz), |
| | | | 7.30(2H, m), 7.38(1H, d, J=16 Hz), |
| | | | 7.60(3H, m), 7.75(1H, m), 7.90(1H, d, |
| | | | J=8 Hz). |
| 73 | 7-CN | trans-CH═CH(3- | Found: 418(MH⁺); C₂₆H₃₁N₃O₂ requires |
| | | furanyl) | 417. |
| | | | (DMSOd₆) δ: 0.85-1.30(5H, m), 1.35(2H, |
| | | | m), 1.80(4H, m), 2.30-2.60(6H, m), 2.85 |
| | | | (4H, m), 3.55(1H, m), 6.28(1H, d, J=16 Hz), |
| | | | 6.66(1H, s), 7.28(1H, d, J=16 Hz), |
| | | | 7.31(1H, d, J=8 Hz), 7.55(2H, m), 7.71 |
| | | | (1H, s), 7.85(1H, d, J=8 Hz), 7.98(1H, s). |
| 74 | 7-CN | trans-CH═CH(4- | Found: 479(MH⁺); C₃₁H₃₄N₄O requires |
| | | quinolinyl) | 478. |
| | | | δ (DMSO + TFA): 1.00-1.30(5H, m), 1.60-1.70 |
| | | | (2H, m), 1.75-1.81(2H, m), 1.90-1.95 |
| | | | (2H, m), 2.90-3.30(8H, m), 3.60-3.80 |
| | | | (2H, m), 7.07(1H, d, J=16 Hz), 7.47 |
| | | | (1H, d, J=8 Hz), 7.71(1H, dd, J=8 Hz), |
| | | | 7.75(1H, s) 7.95(1H, m), 8.05-8.30(4H, |
| | | | m), 8.45(1H, d, J=8 Hz), 8.53(1H, d, J=8 Hz), |
| | | | 9.25(1H, d, J=5 Hz), 9.78(1H, br s). |
| 75 | | trans-CH═CH(5- | Found: 430(MH⁺); C₂₆H₃₁N₅O requires |
| | | pyrimidinyl) | 429. |
| | | | δ (DMSO + TFA): 1.00-1.30(5H, m), 1.55-1.65 |
| | | | (2H, m), 1.75-1.80(2H, m), 1.80-1.90 |
| | | | (2H, m), 3.00-3.25(8H, m), 3.60-3.75 |
| | | | (3H, m), 6.80(1H, d, J=16 Hz), 7.42 |
| | | | (1H, d, J=16 Hz), 7.46(!H, d, J=8 Hz), |
| | | | 7.71(1H, dd, J=8 Hz, 2 Hz), 7.74(1H, s), |
| | | | 8.15(1H, d, J=8 Hz), 9.00(2H, s), 9.15 |
| | | | (1H, s), 9.72(1H, br s). |
| 76 | 7-CN | —CH₂C₆H₃(2,4- | Found: 452(MH⁺); C₂₇H₃₁F₂N₃O |
| | | diF) | requires 451. |
| | | | δ (DMSO + TFA): 0.90-1.10(2H, m), 1.20-1.30 |
| | | | (3H, m), 1.50-1.65(2H, m), 1.70-1.85 |
| | | | (4H, m), 2.90-3.30(8H, m), 3.40(2H, |
| | | | s), 3.50(1H, m), 3.67(2H, m), 7.00(1H, m), |
| | | | 7.15(1H, m), 7.40(1H, m), 7.45(1H, d, J=8 Hz), |
| | | | 7.70(1H, m), 7.73(1H, s), 7.96(1H, |
| | | | d, J=8 Hz), 9.70(1H, br s). |
| 77 | 7-CN | —CH₂(1-naphthyl) | Found: 466(MH⁺); C₃₁H₃₅N₃O requires |
| | | | 465. |
| | | | δ: 0.70-0.80(2H, m), 0.90-1.10(3H, m), |
| | | | 1.30-1.40(2H, m), 1.60-1.70(2H, m), |
| | | | 1.70-1.80(2H, m), 2.40(2H, m), 2.55(4H, |
| | | | m), 2.80-3.00(4H, m), 3.66(1H, m), 4.00 |
| | | | (2H, s), 5.05(1H, d, J=8 Hz), 7.15(1H, d, |
| | | | J=8 Hz), 7.34(1H, s), 7.35-7.40(2H, m), |
| | | | 7.45(1H, m), 7.50(2H, m), 7.83(1H, d, |
| | | | J=8 Hz), 7.86(1H, m), 7.93(1H, m). |
| 78 | 7-CN | —CH₂(6-(2- | Found: 488(MH⁺); C₂₈H₃₃N₅OS requires |
| | | amino)benzo- | 487. |
| | | thiazolyl) | δ: 0.85-1.11(5H, m), 1.37(2H, m), 1.68 |
| | | | (2H, m), 1.89(2H, m), 2.45(2H, m), 2.59 |
| | | | (4H, m), 2.92(4H, m), 3.57(2H, s), 3.68 |
| | | | (1H, m), 5.16(3H, m), 7.17(2H, m), 7.38 |
| | | | (2H, m), 7.52(2H, m). |
| 79 | 7-CN | —CH₂(6-(2- | Found: 487(MH⁺); C₂₉H₃₄N₄OS requires |
| | | methyl)- | 486. |
| | | benzothiazolyl) | δ(DMSO-d₆): 0.93(2H, m), 1.15(3H, m), |
| | | | 1.33(2H, m), 1.75(4H, m), 2.42(2H, m), |
| | | | 2.52(4H, m), 2.77(3H, s), 2.90(4H, m), |
| | | | 3.43(3H, m), 7.33(2H, m), 7.56(2H, m), |
| | | | 7.82(2H, m), 7.95(1H, d, J=8 Hz). |
| 80 | 7-CN | —CH₂(6-(2,3- | Found: 471(MH⁺); C₂₉H₃₄N₄O₂ requires |
| | | dihydro-2-oxo)- | 470. |
| | | indolinyl) | δ (DMSO-d₆ + TFA): 0.88-1.32(5H, m), |
| | | | 1.59(2H, m), 1.74(4H, m), 2.90-3.27(8H, |
| | | | m), 3.29(2H, s), 3.40(2H, s), 3.45(1H, m), |
| | | | 3.65(2H, m), 6.73(1H, s), 6.79(1H, d, J=9 Hz), |
| | | | 7.08(1H, d, J=9 Hz), 7.45(1H, d, |
| | | | J=9 Hz), 7.69(1H, d, J=9 Hz), 7.72(1H, s), |
| | | | 7.90(1H, d, J=9 Hz), 9.86(1H, br s), |
| | | | 10.33(1H, br s). |
| 81 | 7-CN | —CH₂(5-(2,3- | Found: 471(MH⁺); C₂₉H₃₄N₄O₂ requires |
| | | dihydro-2-oxo)- | 470. |
| | | indolinyl) | δ (DMSO-d₆ + TFA): 0.90-1.35(5H, m), |
| | | | 1.59(2H, m), 1.75(4H, m), 2.91-3.29(8H, |
| | | | m), 3.27(2H, s), 3.43(2H, s), 3.47(1H, m), |
| | | | 3.66(2H, m), 6.72(1H, d, J=9 Hz), 7.01 |
| | | | (1H, d, J=9 Hz), 7.06(1H, s), 7.45(1H, d, |
| | | | J=9 Hz), 7.69(1H, d, J=9 Hz), 7.72(1H, |
| | | | s), 7.88(1H, d, J=9 Hz), 9.90(1H, br s), |
| | | | 10.29(1H, br s). |
| 82 | 7-CN | CH═CHC₆H₄(4- | Found: 485(MH⁺); C₃₀H₃₆N₄O₂ requires |
| | | CONHMe) | 484. |
| | | | (DMSO + TFA) δ: 0.97-1.28(5H, m), 1.58-1.64 |
| | | | (2H, m), 1.76-1.90(4H, m), 2.79 |
| | | | (3H, m), 2.99-3.33(8H, m), 3.66-3.77 |
| | | | (3H, m), 6.69(1H, d, J=16 Hz), 7.41-7.45 |
| | | | (2H, m), 7.60-7.73(3H, m), 7.86(2H, d, |
| | | | J=8 Hz), 8.03(1H, d), 8.45(1H, m), 9.75 |
| | | | (1H, b s). |
| 83 | 7-CN | CH₂(5-(2- | Found: 472(MH⁺); C₂₈H₃₃N₅O₂ requires |
| | | amino)benzoxazolyl | 471. |
| | | | (DMSO + TFA) δ: 0.77-1.23(5H, m), 1.54-1.62 |
| | | | (2H, m), 1.70-1.79(4H, m), 2.99-3.24 |
| | | | (8H, m), 3.38-3.45(3H, m), 3.60-3.69 |
| | | | (2H, m), 6.49(1H, d, J=10 Hz), 7.15 |
| | | | (1H, s), 7.32(1H, d, J=8 Hz), 7.45(1H, d, |
| | | | J=8 Hz), 7.67-7.72(2H, m), 7.93(1H, d), |
| | | | 8.31(2H, b s), 9.92(1H, bs). |
| 84 | 7-CN | —CH₂(6-(1,2- | Found: 483(MH⁺); C₃₀H₃₄N₄O₂ requires |
| | | dihydro-2- | 482. |
| | | oxo)quinolinyl) | δ (DMSO + TFA): 0.90-1.20(5H, m), 1.50-1.65 |
| | | | (2H, m), 1.70-1.80(4H, m), 2.90-3.30 |
| | | | (8H, m), 3.40(2H, s), 3.45(1H, m), |
| | | | 3.60-3.70(2H, m), 6.48(1H, d, J=10 Hz), |
| | | | 7.22(1H, d, J=8 Hz), 7.50(1H, dd, J=10 Hz), |
| | | | 2 Hz), 7.60-7.50(2H, m), 7.80(1H, dd, |
| | | | J=10 Hz, 2 Hz), 7.70(1H, s), 7.86(1H, d, |
| | | | J=9 Hz), 7.95(1H, d, J=8 Hz), 9.80(1H, |
| | | | br s), 11.70(1H, br s). |
| 85 | 7-CN | —CH₂(7-(1,2- | Found: 483(MH⁺); C₃₀H₃₄N₄O₂ requires |
| | | dihydro-2- | 482. |
| | | oxo)quinolinyl) | δ (DMSO): 0:90-1.00(2H, ,m), 1.10-1.20 |
| | | | (5H, m), 1.25-1.40(2H, m), 1.70-1.80 |
| | | | (4H, m), 2.40-2.50(4H, m), 2.80-2.90 |
| | | | (4H, m), 3.30-3.45(3H, m), 6.43(1H, d, |
| | | | J=8 Hz), 7.05(1H, d, J=8 Hz), 7.17(1H, s), |
| | | | 7.32(1H, d, J=8 Hz), 7.50-7.60(3H, m), |
| | | | 7.84(1H, d, J=9 Hz), 7.97(1H, d, J=8 Hz), |
| | | | 11.70(1H, s). |
| 86 | 7-CN | —C₆H₄(3-(1- | Found: 468(MH⁺); C₂₉H₃₃N₅O requires |
| | | pyrazolyl)) | 467. |
| | | | δ: 1.06-1.40(5H, m), 1.40-1.50(2H, m), |
| | | | 1.81-1.86(2H, m), 2.08-2.13(2H, m), |
| | | | 2.38-2.54(2H, m), 2.61-2.65(4H, m), |
| | | | 2.90-3.00(4H, m), 3.89-3.96(1H, m), |
| | | | 6.06(1H, d, J=8 Hz), 6.50(1H, t, J=2 Hz), |
| | | | 7.18(1H, d, J=8 Hz), 7.29-7.43(2H, |
| | | | m), 7.51(1H, t, J=8 Hz), 7.68(1H, d, J=8 Hz), |
| | | | 7.75(1H, d, J=1.5 Hz), 7.79-7.83 |
| | | | (1H, m), 8.00(1H, m), 8.08(1H, m). |
| 87 | 7-CN | —CH₂(2- | Found: 422(MH⁺); C₂₅H₃₁N₃OS requires |
| | | thiophenyl) | 421. |
| | | | δ: 0.90-1.20(5H, m), 1.35-1.46(2H, m), |
| | | | 1.69-1.72(2H, m), 1.80-2.00(2H, m), |
| | | | 2.42-2.48(2H, m), 2.57-2.65(4H, m), |
| | | | 2.90-2.96(4H, m), 3.65-3.75(1H, m), |
| | | | 3.74(2H, s), 5.38(1H, d, J=8 Hz), 8.92 |
| | | | (1H, m), 6.98(1H, m), 7.16(1H, d, J=8 Hz), |
| | | | 7.23-7.26(1H, m), 7.36-7.42(2H, |
| | | | m). |
| 88 | 7-CN | —CH₂(3- | Found: 472(MH⁺); C₂₉H₃₃N₃OS requires |
| | | benzothiophenyl) | 471. |
| | | | δ: 0.80-1.20(5H, m), 1.30-1.40(2H, m), |
| | | | 1.60-1.75(2H, m), 1.80-1.90(2H, m), |
| | | | 2.40-2.50(2H, m), 2.50-2.70(4H, m), |
| | | | 2.85-2.95(4H, m), 3.65-3.75(1H, m), |
| | | | 3.80(2H, s), 5.23(1H, d, J=8 Hz), 7.16 |
| | | | (1H, d, J=8 Hz), 7.30-7.45(5H, m), |
| | | | 7.60-7.70(1H, m), 7.85-7.92(1H, m). |
| 89 | 7-CN | —C₆H₄(3-(2-(5- | Found: 484(MH⁺); C₂₉H₃₃N₅O₂ requires |
| | | methyl)-1,3,4- | 483. |
| | | oxadiazolyl) | δ: 1.10-1.40(5H, m), 1.41-1.50(2H, m), |
| | | | 1.82-1.87(2H, m), 2.10-2.14(2H, m), |
| | | | 2.48-2.54(2H, m), 2.62-2.65(7H, m), |
| | | | 2.93-3.00(4H, m), 3.93-3.97(1H, m), |
| | | | 6.04(1H, d, J=8 Hz), 7.18(1H, d, J=8 Hz), |
| | | | 7.35-7.43(2H, m), 7.59(1H, t, J=8 Hz), |
| | | | 7.97(1H, dd, J=6 and 1 Hz), 8.11 |
| | | | (1H, dd, J=8 and 1 Hz), 8.36(1H, d, |
| | | | J=1 Hz). |
| 90 | 7-CN | trans-CH═CH(2- | Found: 478(MH⁺); C₃₂H₃₅N₃O requires |
| | | naphthyl) | 477. |
| | | | δ (DMSO + TFA): 1.00-1.65(5H, m), 1.63 |
| | | | (2H, m), 1.76-1.91(4H, m), 2.97-3.32 |
| | | | (8H, m), 3.66-3.93(3H, m), 6.75(1H, d, |
| | | | J=16 Hz), 7.47(1H, d), 7.54-7.62(3H, m), |
| | | | 7.65-7.80(3H, m), 7.85-7.95(3H, m), |
| | | | 8.00-8.10(2H, m), 9.77(1H, b s). |
| 91 | 7-CN | trans-CH═CH(5- | Found: 509(MH⁺); C₃₂H₃₆N₄O₂ requires |
| | | (3-acetyl)indolyl | 508. |
| | | | δ (DMSOd₆): 0.85-1.28(5H, m), 1.35(2H, |
| | | | m), 1.65-1.95(4H, m), 2.35-2.65(6H, m), |
| | | | 2.46(3H, s), 2.91(4H, m), 3.59(1H, m), |
| | | | 6.60(1H, d, J=16 Hz), 7.30-7.65(6H, m), |
| | | | 7.96(1H, d, J=8 Hz), 8.37(2H, m), 12.05 |
| | | | (1H, br s). |
| 92 | 7-CN | —C₆H₄(5-(3- | Found: 484(MH⁺); C₂₉H₃₃N₅O₂ requires |
| | | methyl)-1,2,4- | 483. |
| | | oxadiazolyl | δ (CDCl₃): 1.13-1.28(5H, m), 1.43-1.48 |
| | | | (2H, m), 1.83-1.86(2H, m), 2.10-2.13 |
| | | | (2H, m), 2.49(3H, s), 2.51(2H, m), 2.62-2.64 |
| | | | (4H, m), 2.88-2.98(4H, m), 3.94-3.98 |
| | | | (1H, m), 6.02(1H, d, J=8 Hz), 7.18 |
| | | | (1H, d, J=7.7 Hz), 7.38(!H, s), 7.39(1H, d, |
| | | | H = 7.7 Hz), 7.64(1H, t, J=7.8 Hz), 8.05 |
| | | | (1H, d), 8.21(1H, d), 8.39(1H, br s). |
| 93 | 7-CN | —CH₂(5-(2- | Found: 470(MH⁺); C₂₉H₃₅N₅O requires |
| | | methyl)- | 469. |
| | | benzimidazolyl) | δ: 0.87-1.09(5H, m), 1.14(1H, br s), 1.37 |
| | | | (2H, m), 1.70(2H, m), 1.88(2H, m), 2.45 |
| | | | (2H, m), 2.56(3H, s), 2.60(4H, m), 2.93 |
| | | | (4H, m), 3.61(2H, s), 3.69(1H, m), 3.61 |
| | | | (2H, s), 3.69(1H, m), 5.44(1H, d, J=7 Hz), |
| | | | 7.04(1H, dd, J=8.2 Hz), 7.15(1H, d, |
| | | | J=8 Hz), 7.30-7.47(4H, m). |
| 94 | 7-CN | —CH₂(6- | Found: 468(MH⁺); C₂₉H₃₃N₅O requires |
| | | quinoxalinyl) | 467. |
| | | | δ: 0.90-1.15(5H, m), 1.40(2H, m), 1.73 |
| | | | (2H, m), 1.93(2H, m), 2.45(2H, m), 2.59 |
| | | | (4H, m), 2.94(4H, m), 3.67(1H, m), 3.76 |
| | | | (2H, s), 5.33(1H, d, J=7 Hz), 7.16(1H, d, |
| | | | J=8 Hz), 7.31-7.44(2H, m), 7.72(1H, dd, |
| | | | J=9.2 Hz), 7.96(1H, d, J=2 Hz), 8.08 |
| | | | (1H, d, J=9 Hz), 8.85(2H, s). |
| 95 | 7-CN | trans-CH═CH(3- | Found: 460(MH⁺); C₂₈H₃₃N₃O₃ requires |
| | | (2-acetyl)furanyl) | 459. |
| | | | δ: 1.05-1.35(5H, m), 1.45(2H, m), 1.80 |
| | | | (2H, m), 2.04(2H, m), 2.48(2H, m), 2.53 |
| | | | (3H, s), 2.61(4H, m), 2.95(4H, m), 3.84 |
| | | | (1H, m), 5.56(1H, d, J=8 Hz), 6.43(1H, d, |
| | | | J=16 Hz), 6.70(1H, d, J=2 Hz), 7.17(1H, |
| | | | d, J=8 Hz), 7.38(1H, s), 7.41(1H, d, J=8 Hz), |
| | | | 7.45(1H, d, J=2 Hz), 7.95(1H, d, |
| | | | J=16 Hz). |
| 96 | 7-CN | —CH₂(6-(2-amino) | Found: 472(MH⁺); C₂₈H₃₃N₅O₂ requires |
| | | benzoxazolyl) | 471. |
| | | | δ: 0.81-1.12(5H, m), 1.40(2H, m), 1.72 |
| | | | (2H, m), 1.89(2H, m), 2.45(2H, m), 2.93 |
| | | | (4H, m), 3.58(2H, s), 3.69(1H, m), 4.92 |
| | | | (2H, br s), 5.13(1H, m), 7.05(1H, m), 7.17 |
| | | | (2H, m), 7.36(3H, m). |
| 97 | 7-CN | —CH₂(6-(3,4- | Found: 487(MH⁺); C₂₉H₃₄N₄O₃ requires |
| | | dihydro-2-oxo)- | 486. |
| | | 2H-benzoxazinyl) | δ (DMSO-d₆): 0.83-1.24(5H, m), 1.32 |
| | | | (2H, m), 1.73(4H, m), 2.47(6H, m), 2.90 |
| | | | (4H, m), 3.25(2H, s), 3.40(1H, m), 4.53 |
| | | | (2H, s), 6.80(3H, m), 7.31(1H, d, J=8 Hz), |
| | | | 7.56(2H, m), 7.84(1H, d, J=8 Hz), |
| | | | 10.62(1H, br s). |
| 98 | 7-CN | trans- | Found: 503(MH⁺); C₃₀H₃₅FN₄O₂ |
| | | CH═CHC₆H₃(2- | requires 502. |
| | | F, 5-NHCOMe) | δ (DMSO-d₆ + TFA): 0.94-1.34(5H, m), |
| | | | 1.61(2H, m), 1.76(2H, m), 1.87(2H, m), |
| | | | 2.05(3H, s), 2.93-3.33(8H, m), 3.54-3.77 |
| | | | (3H, m), 6.65(1H, d, J=15 Hz), 7.20 |
| | | | (1H, t, J=9 Hz), 7.43(3H, m), 7.69(1H, d, |
| | | | J=9 Hz), 7.73(1H, s), 8.03(1H, m), 8.19 |
| | | | (1H, d, J=9 Hz), 9.88(1H, br s), 10.09 |
| | | | (1H, brs). |

TABLE 2

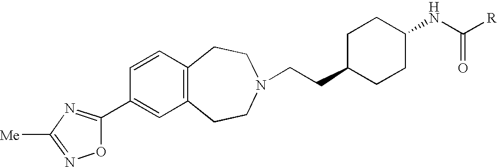

| Example | | |
|---|---|---|
| No. | R | Mass spectrum, ¹H NMR |
| 99 | —CH₂-(2-benzothiophenyl) | Mass spectrum(API⁺): Found 529(MH⁺). |
| | | C₃₁H₃₆N₄O₂S requires 528. |
| | | NMR(CDCl₃) δ: 1.00-1.10(4H, m), 1.19 |
| | | (1H, m), 1.35-1.45(2H, m), 1.75(2H, m), |
| | | 1.95(2H, m), 2.40-2.50(5H, m), 2.61 |
| | | (4H, m), 2.97(4H, m), 3.73(1H, m), 3.82 |
| | | (2H, s), 5.46(1H, d, J=8 Hz), 7.16(1H, |
| | | s), 7.22(1H, d, J=8 Hz), 7.25-7.42(2H, |
| | | m), 7.73(1H, d, J=8 Hz), 7.79(1H, d, |
| | | J=8 Hz), 7.81-7.88(2H, m). |
| 100 | (E)-CH═CH-(3-thienyl) | Mass spectrum(API⁺): Found 491(MH⁺). |
| | | C₂₈H₃₄N₄O₂S requires 490. |
| | | NMR(CDCl₃) δ: 1.04-1.15(4H, m), 1.25 |
| | | (1H, m), 1.44(2H, m), 1.76(2H, m), 2.05 |
| | | (2H, m), 2.46(3H, s), 2.50(2H, m), 2.64 |
| | | (4H, m), 3.00(4H, m), 3.85(1H, m), 5.36 |
| | | (1H, d, J=8 Hz), 6.18(1H, d, J=16 Hz), |
| | | 7.22-7.20(2H, m), 7.30(1H, m), 7.43 |
| | | (1H, m), 7.59(1H, d, J=16 Hz), |
| | | 7.80-7.90(2H, m). |
| 101 | 5-quinolyl | Mass spectrum(API⁺): Found |
| | | 510(MH⁺). C₃₁H₃₅N₅O₂ requires 509. |
| | | NMR(CDCl₃) δ: 1.15-1.27(5H, m), 1.45 |
| | | (2H, m), 1.85(2H, m), 2.20(2H, m), 2.46 |
| | | (3H, s), 2.55(2H, m), 2.70(4H, m), 3.00 |
| | | (4H, m), 4.00(1H, m), 5.85(1H, d, J=8 Hz), |
| | | 7.25(1H, d, J=8 Hz), 7.46(1H, dd, |
| | | J=4.8 Hz), 7.60-7.72(2H, m), 7.84-7.87 |
| | | (2H, m), 8.16(1H, d, J=8 Hz), 8.72(1H, |
| | | d, J=8 Hz), 8.92(1H, m). |
| 102 | 3-pyrrolo[2,3-b]pyridyl | Mass spectrum(API⁺): Found 499(MH⁺). |
| | | C₂₉H₃₆N₆O₂ requires 498. |
| | | NMR(DMSO-d₆) δ: 0.90-1.10(2H, m), |
| | | 1.10-1.40(5H, m), 1.70-1.90(4H, m), |
| | | 2.40-2.70(6H, m), 2.96(3H, s), 3.31(4H, |
| | | m), 3.89(1H, m), 7.15(1H, m), 7.36(1H, |
| | | d, J=8 Hz), 7.71(1H, d, J=8 Hz), 7.75-7.85 |
| | | (2H, m), 8.12(1H, s), 8.20(1H, s), |
| | | 8.35(1H, d, J=8 Hz), 12.02(1H, br s). |
| 103 | 3-(3-(5-methyl)-1,2,4- | Mass spectrum(API⁺): Found 541(MH⁺). |
| | oxadiazolyl)phenyl | C₃₁H₃₆N₆O₃ requires 540. |
| | | NMR(CDCl₃) δ 1.10-1.22(4H, m),1.27 |
| | | (1H, m), 1.55(2H, m), 1.90(2H, m), 2.10 |
| | | (2H, m), 2.47(3H, s), 3.65(2H, m), 2.68 |
| | | (3H, s), 2.76(4H, m), 3.06(4H, m), 3.95 |
| | | (1H, m), 6.00(1H, d, J=8 Hz), 7.25(1H, |
| | | d, J=8 Hz), 7.57(1H, t, J=8 Hz), 7.80-7.90 |
| | | (2H, m), 8.02(1H, d, J=8 Hz), 8.15 |
| | | (1H, d, J=8 Hz), 8.32(1H, s). |
| 104 | 8-(1,4-dihydro-4- | Mass spectrum(API⁺): Found 526(MH⁺). |
| | oxo)quinolyl | C₃₁H₃₅N₅O₃ requires 525. |
| | | NMR(DMSO-d₆) δ: 0.90-1.10(2H, m), |
| | | 1.20-1.40(5H, m), 1.80-2.00(4H, m), |
| | | 2.30-2.75(9H, m), 2.96(4H, m), 3.80 |
| | | (1H, m), 6.09(1H, d, J=8 Hz), 7.30-7.40 |
| | | (2H, m), 7.75-7.88(2H, m), 7.92(1H, m), |
| | | 8.05(1H, d, J=8 Hz), 8.22(1H, d, J=8 Hz), |
| | | 8.65(1H, d, J=8 Hz), 12.04(1H, br |
| | | s). |
| 105 | (E)-CH═CH-(4- | Mass spectrum(API⁺): Found 503(MH⁺). |
| | fluoro)phenyl | C₃₀H₃₅FN₄O₂ requires 502. |
| | | NMR(CDCl₃) δ: 1.10-1.30(5H, m), 1.40-1.47 |
| | | (2H, m), 1.78-1.82(2H, m), 2.00-2.10 |
| | | (2H, m), 2.46(3H, s), 2.47-2.52(2H, |
| | | m), 2.60-2.70(4H, m), 2.95-3.05(4H, |
| | | m), 3.86(1H, m), 5.38(1H, d, J=8 Hz), |
| | | 6.26(1H, d, J=16 Hz), 7.05(2H, t, J=8 Hz), |
| | | 7.24(1H, d, J=8 Hz), 7.47(2H, dd, |
| | | J=5.8 Hz), 7.57(1H, d, J=16 Hz), |
| | | 7.80-7.90(2H, m). |
| 106 | (E)-CH═CH-(3- | Mass spectrum(API⁺): Found 503(MH⁺). |
| | fluoro)phenyl | C₃₀H₃₅FN₄O₂ requires 502. |
| | | NMR(CDCl₃) δ: 1.10-1.30(5H, m), 1.42 |
| | | (2H, m), 1.81(2H, m), 2.06(2H, m), 2.46 |
| | | (3H, s), 2.51(2H, m), 2.65(4H, m), 3.00 |
| | | (4H, m), 3.87(1H, m), 5.41(1H, d, J=8 Hz), |
| | | 6.33(1H, d, J=16 Hz), 7.02(1H, m), |
| | | 7.15(1H, m), 7.25(2H, m), 7.31(1H, m), |
| | | 7.57(1H, d, J=16 Hz), 7.80-7.90(2H, |
| | | m). |
| 107 | (E)-CH═CH-(3-acetamido- | Mass spectrum(API⁺): Found 560(MH⁺). |
| | 2-fluoro)phenyl | C₃₂H₃₈FN₅O₃ requires 559. |
| | | NMR(CDCl₃) δ: 1.10-1.20(4H, m), 1.20-1.30 |
| | | (1H, m), 1.40-1.50(2H, m), 1.77-1.83 |
| | | (2H, m), 2.05-2.12(2H, m), 2.24 |
| | | (3H, s), 2.46(3H, s), 2.55(2H, m), 2.65 |
| | | (4H, m), 3.00(4H, m), 3.85(1H, m), 5.42 |
| | | (1H, d, J=8 Hz), 6.42(1H, d, J=16 Hz), |
| | | 7.12(1H, t, J=8 Hz), 7.18-7.30(2H, m), |
| | | 7.38(1H, s), 7.71(1H, d, J=16 Hz), |
| | | 7.80-7.90(2H, m), 8.30(1H, m). |
| 108 | (E)-CH═CH-(3- | Mass spectrum(API⁺): Found 527(MH⁺). |
| | acetyl)phenyl | C₃₂H₃₈N₄O₃ requires 526. |
| | | NMR(CDCl₃) δ: 1.10-1.20(4H, m), 1.20-1.30 |
| | | (1H, m), 1.50(2H, m), 1.80(2H, m), |
| | | 2.05(2H, m), 2.46(3H, s), 2.56(2H, m), |
| | | 2.60(3H, s), 2.65(4H, m), 3.00(4H, m), |
| | | 3.85(1H, m), 5.48(1H, d, J=8 Hz), 6.20 |
| | | (1H, d, J=16 Hz), 7.24(1H, d, J=8 Hz), |
| | | 7.40(1H, m), 7.45-7.55(2H, m), 7.70 |
| | | (1H, d, J=8 Hz), 7.85-7.88(2H, m), 7.91 |
| | | (1H, d, J=16 Hz). |
| 109 | —CH₂-(3-fluoro)phenyl | Mass spectrum(API⁺): Found 491(MH⁺). |
| | | C₂₉H₃₅FN₄O₂ requires 490. |
| | | NMR(CDCl₃) δ: 1.00-1.12(4H, m), 1.19 |
| | | (1H, m), 1.40(2H, m), 1.75(2H, m), 1.93 |
| | | (2H, m), 2.40-2.50(5H, m), 2.62(4H, m), |
| | | 2.95(4H, m), 3.52(2H, s), 3.70(1H, m), |
| | | 5.14(1H, d, J=8 Hz), 6.90-7.05(3H, m), |
| | | 7.22(1H, d, J=8 Hz), 7.30(1H, m), |
| | | 7.80-7.90(2H, m). |
| 110 | —CH₂-(2,4-difluoro)phenyl | Mass spectrum(API⁺): Found 509(MH⁺). |
| | | C₂₉H₃₄F₂N₄O₂ requires 508. |
| | | NMR(CDCl₃) δ: 1.00-1.10(4H, m), 1.15-1.25 |
| | | (1H, m), 1.35-1.45(2H, m), 1.70-1.80 |
| | | (2H, m), 1.90-2.00(2H, m), 2.46 |
| | | (3H, s), 2.48(2H, m), 2.63(4H, m), 2.97 |
| | | (4H, m), 3.48(2H, s), 3.70(1H, m), 5.24 |
| | | (1H, d, J=8 Hz), 6.85(2H, m), 7.23(1H, |
| | | d, J=8 Hz), 7.24-7.35(1H, m), |
| | | 7.80-7.90(2H, m). |
| 111 | 2-naphthyl | Mass spectrum(API⁺): Found 509(MH⁺). |
| | | C₃₂H₃₆N₄O₂ requires 508. |
| | | NMR(CDCl₃) δ: 1.10-1.35(5H, m), 1.40-1.50 |
| | | (2H, m), 1.80-1.90(2H, m), 2.10-2.20 |
| | | (2H, m), 2.46(3H, s), 2.55(2H, m), |
| | | 2.67(4H, m), 3.01(4H, m), 4.00(1H, m), |
| | | 6.04(1H, d, J=8 Hz), 7.24(1H, d, J=8 Hz), |
| | | 7.55(2H, m), 7.80-7.95(6H, m), |
| | | 8.25(1H, s). |
| 112 | 7-(3,4-dihydro-3-oxo)-2H- | Mass spectrum(API⁺): Found 530(MH⁺). |
| | benzoxazinyl | C₃₀H₃₅N₅O₄ requires 529. |
| | | NMR(CDCl₃) δ: 1.10-1.30(5H, m), 1.40-1.50 |
| | | (2H, m), 1.75-1.85(2H, m), 2.00-2.10 |
| | | (2H, m), 2.46(3H, s), 2.50-2.60(2H, |
| | | m), 2.64-2.75(4H, m), 2.95-3.05(4H, |
| | | m), 3.90(1H, m), 4.64(2H, s), 5.79(1H, d, |
| | | J=8 Hz), 6.81(1H, d, J=8 Hz), 7.20-7.22 |
| | | (1H, m), 7.40(2H, m), 7.72(1H, br s), |
| | | 7.83-7.90(2H, m). |
| 113 | 5-quinolinyl(2-Me) | Mass spectrum(API⁺): Found 524(MH⁺). |
| | | C₃₂H₃₇N₅O₂ requires 523. |
| | | NMR(DMSO-d₆) δ: 1.02-1.10(2H, m), |
| | | 1.20-1.40(5H, m), 1.75-1.83(2H, m), |
| | | 1.90-2.00(2H, m), 2.33(2H, m), 2.40 |
| | | (3H, s), 2.55-2.60(4H, m), 2.66(3H, s), |
| | | 2.90-3.00(4H, m), 3.75-3.85(1H, s), |
| | | 7.35-7.37(1H, m), 7.44-7.47(1H, m), |
| | | 7.57-7.59(1H, m), 7.69-7.72(1H, m), |
| | | 7.81-7.85(2H, m), 7.96-8.00(1H, m), |
| | | 8.41-8.48(2H, m). |
| 114 | —CH₂-(2-fluoro)phenyl | Mass spectrum(API⁺): Found 491(MH⁺). |
| | | C₂₉H₃₅FN₄O₂ requires 490. |
| | | NMR(CDCl₃) δ: 1.00-1.07(4H, m), 1.18-1.23 |
| | | (1H, m), 1.38-1.43(2H, m), 1.72-1.76 |
| | | (2H, m), 1.91-1.94(2H, m), 2.46 |
| | | (3H, s), 2.45-2.49(2H, m), 2.60-2.64 |
| | | (4H, m), 2.95-2.99(4H, m), 3.54(2H, s), |
| | | 3.67-3.72(1H, m), 5.25(1H, d, J=8 Hz), |
| | | 7.04-7.14(2H, m), 7.21-7.32(3H, m), |
| | | 7.84-7.86(2H, m). |
| 115 | —CH₂-(2,5-difluoro)phenyl | Mass spectrum(API⁺): Found 509(MH⁺). |
| | | C₂₉H₃₄F₂N₄O₂ requires 508. |
| | | NMR(CDCl₃) δ: 0.96-1.29(5H, m), 1.37-1.46 |
| | | (2H, m), 1.67-1.79(2H, m), 1.93-1.97 |
| | | (2H, m), 2.46(3H, s), 2.44-2.50 |
| | | (2H, m), 2.60-2.65(4H, m), 2.95-3.05 |
| | | (4H, m), 3.50(2H, s), 3.62-3.76(1H, m), |
| | | 5.30(1H, d, J=8 Hz), 6.89-7.08(2H, m), |
| | | 7.21-7.24(2H, m), 7.84-7.87(2H, m). |
| 116 | 2-indolyl | Mass spectrum(API⁺): Found 498(MH⁺). |
| | | C₃₀H₃₅N₅O₂ requires 497. |
| | | NMR(DMSO-d₆) δ: 0.97-1.11(2H, m), |
| | | 1.26-1.50(5H, m), 1.70-2.00(4H, m), |
| | | 2.39-2.62(5H, m), 2.93-3.02(4H, m), |
| | | 3.31-3.40(4H, m), 3.70-3.90(1H, m), |
| | | 6.98-7.04(1H, m), 7.13-7.18(2H, m), |
| | | 7.35-7.43(2H, m), 7.59(1H, d, J=8 Hz), |
| | | 7.81-7.85(2H, m), 8.20(1H, d, J=8 Hz), |
| | | 11.50-11.54(1H, s). |

TABLE 3

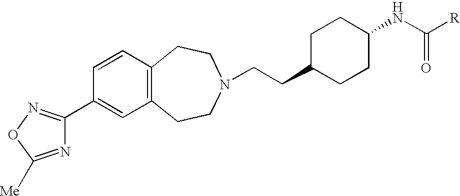

| Example | | |
|---|---|---|
| No. | R | Mass spectrum, ¹H NMR |
| 117 | —CH₂-(2-benzothiophenyl) | Mass spectrum (API⁺): |
| | | Found 529 (MH⁺). |
| | | C₃₁H₃₆N₄O₂S requires 528. |
| | | NMR (CDCl₃) δ: 0.95-1.10 |
| | | (4H, m), 1.18 (1H, m), 1.35- |
| | | 1.45 (2H, m), 1.74 (2H, m), |
| | | 1.95 (2H, m), 2.45 (2H, m), |
| | | 2.55-2.68 (4H, m), 2.64 |
| | | (3H, s), 2.90-3.00 (4H, m), |
| | | 3.73 (1H, m), 3.82 (2H, s), |
| | | 5.46 (1H, d, J=8 Hz), 7.16 |
| | | (1H, s), 7.18 (1H, d, J=8 Hz), |
| | | 7.27-7.40 (2H, m), 7.72 (1H, d, |
| | | J=7 Hz), 7.76-7.85 (3H, m). |
| 118 | (E)-CH═CH-(3-thienyl) | Mass spectrum (API⁺): |
| | | Found 491 (MH⁺). |
| | | C₂₈H₃₄N₄O₂S requires 490. |
| | | NMR (CDCl₃) δ: 1.05-1.20 |
| | | (4H, m), 1.24 (1H, m), 1.44 |
| | | (2H, m), 1.80 (2H, m), 2.05 |
| | | (2H, m), 2.50 (2H, m), 2.55- |
| | | 2.70 (7H, m), 2.90-3.05 (4H, |
| | | m), 3.85 (1H, m), 5.35 (1H, |
| | | d, J=8 Hz), 6.18 (1H, d, |
| | | J=16 Hz), 7.19 (1H, d, J= |
| | | 8 Hz), 7.21-7.27 (1H, m), |
| | | 7.32 (1H, m), 7.43 (1H, m), |
| | | 7.59 (1H, d, J=16 Hz), 7.75- |
| | | 7.85 (2H, m). |
| 119 | 5-quinolinyl | Mass spectrum (API⁺): |
| | | Found 510 (MH⁺). |
| | | C₃₁H₃₅N₅O₂ requires 509. |
| | | NMR (CDCl₃) δ: 1.10-1.35 |
| | | (5H, m), 1.48 (2H, m), 1.80- |
| | | 1.90 (2H, m), 2.10-2.25 (2H, |
| | | m), 2.53 (2H, m), 2.65 (3H, s), |
| | | 2.60-2.70 (4H, m), 2.99 (4H, |
| | | m), 4.03 (1H, m), 5.85 (1H, d, |
| | | J=8 Hz), 7.20 (1H, d, J=8 |
| | | Hz), 7.46 (1H, dd, J=4, 8 Hz), |
| | | 7.66 (2H, m), 7.78-7.85 (2H, |
| | | m), 8.16 (1H, d, J=8 Hz), 8.74 |
| | | (1H, d, J=8 Hz), 8.95 (1H, m). |
| 120 | 3-pyrrolo[2,3-b]pyridyl | Mass spectrum (API⁺): |
| | | Found 499 (MH⁺). |
| | | C₂₉H₃₄N₆O₂ requires 498. |
| | | NMR (DMSO-d₆) δ: 0.90-1.10 |
| | | (2H, m), 1.20-1.50 (5H, m), |
| | | 1.70-1.90 (4H, m), 2.40-2.60 |
| | | (6H, m), 2.65 (3H, s), 2.93 (4H, |
| | | m), 3.75 (1H, m), 7.14 (1H, dd, |
| | | J=4, 8 Hz), 7.29 (1H, d, J=8 |
| | | Hz), 7.60-7.80 (3H, m), 8.14 |
| | | (1H, s), 8.23 (1H, m), 8.43 (1H, |
| | | m), 11.99 (1H, s). |
| 121 | 8-(1,4-dihydro-4- | Mass spectrum (API⁺): |
| | oxo)quinolyl | Found 526 (MH⁺). |
| | | C₃₁H₃₅N₅O₃ requires 525. |
| | | NMR (CDCl₃) δ: 1.10-1.20 |
| | | (2H, m), 1.20-1.34 (3H, m), |
| | | 1.42-1.50 (2H, m), 1.80- |
| | | 1.90 (2H, m), 2.05-2.15 (2H, |
| | | m), 2.50 (2H, m), 2.65 (3H, s), |
| | | 2.65-2.70 (4H, m), 2.98 (4H, |
| | | m), 3.95 (1H, m), 6.30 (1H, d, |
| | | J=8 Hz), 6.33 (1H, dd, J=2, |
| | | 8 Hz), 7.20 (1H, d, J=8 Hz), |
| | | 7.31 (1H, t, J=8 Hz), 7.67 |
| | | (1H, t, J=8 Hz), 7.81 |
| | | (3H, m), 8.55 (1H, d, J=8 Hz), |
| | | 12.20 (1H, br s). |
| 122 | 3-(3-(5-methyl)-1,2,4- | Mass spectrum (API⁺): |
| | oxadiazolyl)phenyl | Found 541 (MH⁺). |
| | | C₃₁H₃₆N₆O₃ requires 540. |
| | | NMR (CDCl₃) δ: 1.10-1.30 |
| | | (5H, m), 1.40 (2H, m), 1.83 |
| | | (2H, m), 2.10 (2H, m), 2.52 |
| | | (2H, m), 2.60-2.70 (10H, m), |
| | | 2.98 (4H, m), 3.96 (1H, m), |
| | | 6.00 (1H, d, J=8 Hz), 7.20 |
| | | (1H, d, J=8 Hz), 7.57 (1H, |
| | | t, J=8 Hz), 7.75-7.82 |
| | | (2H, m), 7.97 (1H, d, J=8 Hz), |
| | | 8.17 (1H, d, J=8 Hz), 8.32 |
| | | (1H, s). |
| 123 | (E)-CH═CH(4- | Mass spectrum (API⁺): |
| | fluoro)phenyl | Found 503 (MH⁺). |
| | | C₃₀H₃₅FN₄O₂ requires 502. |
| | | NMR (CDCl₃) δ: 1.10- |
| | | 1.80 (4H, m), 1.25 (1H, m), |
| | | 1.44 (2H, m), 1.78 (2H, m), |
| | | 2.06 (2H, m), 2.50 (2H, m), |
| | | 2.60-2.70 (7H, m), 2.90-3.00 |
| | | (4H, m), 3.85 (1H, m), 5.39 |
| | | (1H, d, J=8 Hz), 6.26 (1H, d, J= |
| | | 16 Hz), 7.05 (2H, t, J=8 Hz), |
| | | 7.20 (1H, d, J=8 Hz), 7.47 (2H, |
| | | m), 7.57 (1H, d, J=16 Hz), |
| | | 7.80-7.90 (2H, m). |
| 124 | (E)-CH═CH-(3-F)phenyl | Mass spectrum (API⁺): |
| | | Found 503 (MH⁺). |
| | | C₃₀H₃₅FN₄O₂ requires 502. |
| | | NMR (CDCl₃) δ: 1.05-1.20 |
| | | (4H, m), 1.20-1.30 (1H, m), |
| | | 1.40-1.50 (2H, m), 1.75- |
| | | 1.85 (2H, m), 2.00-2.10 (2H, |
| | | m), 2.45-2.55 (2H, m), 2.60- |
| | | 2.70 (7H, m), 2.90-3.05 (4H, |
| | | m), 3.80-3.90 (1H, m), 5.41 |
| | | (1H, d, J=8 Hz), 6.33 (1H, d, J= |
| | | 15 Hz), 6.95-7.05 (1H, m), |
| | | 7.13-7.20 (2H, m), 7.20-7.25 |
| | | (1H, m), 7.27-7.35 (1H, m), |
| | | 7.56 (1H, d, J=15 Hz), 7.75- |
| | | 7.85 (2H, m). |
| 125 | (E)-CH═CH-(2-F)phenyl | Mass spectrum (API⁺): |
| | | Found 503 (MH⁺). |
| | | C₃₀H₃₅FN₄O₂ requires 502. |
| | | NMR (CDCl₃) δ: 1.06-1.30 |
| | | (5H, m), 1.40-1.50 (2H, m), |
| | | 1.75-1.85 (2H, m), 2.00- |
| | | 2.10 (2H, m), 2.45-2.55 (2H, |
| | | m), 2.60-2.70 (7H, m), 2.90- |
| | | 3.00 (4H, m), 3.80-3.90 (1H, |
| | | m), 5.42 (1H, d, J=8 Hz), |
| | | 4.49 (1H, d, J=15 Hz), 7.10- |
| | | 7.22 (3H, m), 7.26-7.31 (1H, |
| | | m), 7.40-7.50 (1H, m). |
| | | 7.66 (1H, d, J=15 Hz), 7.75- |
| | | 7.85 (2H, m). |

TABLE 4

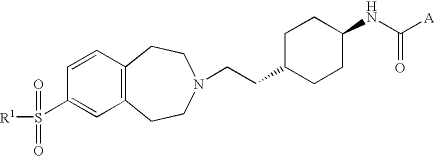

| Ex- | | | |
|---|---|---|---|
| ample | R¹ | A | Mass spectrum |
| 126 | Me | trans-CH═CHC₆H₄(2-F) | Found: 499 (MH⁺) |
| | | | C₂₈H₃₅N₂SO₃F requires 498 |
| 127 | Me | —C₆H₄(3-(2-(4-Methyl)- | Found: 536 (MH⁺) |
| | | oxazolyl)) | C₃₀H₃₇N₃SO₄ requires 535 |
| 142 | Me | —C₆H₄(3-trifluoromethyl) | Found: 523 |
| | | | (MH⁺)C₂₇H₃₃N₂SO₃F₃ |
| | | | requires 522 |
| 129 | Me | 5-quinolinyl(8-Cl, 2-Me) | Found: 554 (MH⁺) |
| | | | C₃₀H₃₆N₃SO₃Cl requires 553 |
| 130 | Me | 5-quinolinyl(8-F, 2-Me) | Found: 538 (MH⁺) |
| | | | C₃₀H₃₆FN₃O₃S requires 537 |

The substituted benzazepines required as intermediates for the compounds of Table 5 were prepared from the compounds of Descriptions 8, 9, or 13, using standard methods for functional group transformation and heterocyclic ring synthesis or by palladium-catalysed cross-coupling reactions.

TABLE 5

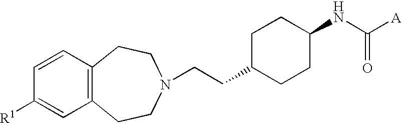

| Example | R¹ | A | Mass Spectrum (API+) |
|---|---|---|---|
| 131 | 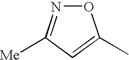 | —CH₂Ph(2-F) | Found 490 (MH⁺). C₃₀H₃₆FN₃O₂requires 489. |
| 132 | 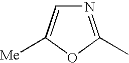 | 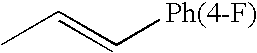 | Found 502 (MH⁺). C₃₁H₃₆FN₃O₂requires 501. |
| 133 | 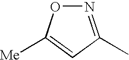 | 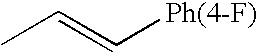 | Found 502 (MH⁺). C₃₁H₃₆FN₃O₂requires 501. |
| 134 | 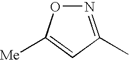 | —CH₂Ph(4-F) | Found 490 (MH⁺). C₃₀H₃₆FN₃O₂requires 489. |
| 135 | 2-pyridyl | 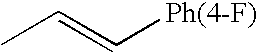 | Found 498 (MH⁺). C₃₂H₃₆FN₃O requires 497. |
| 136 | 2-pyrimidinyl | 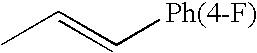 | Found 499 (MH⁺). C₃₁H₃₅FN₄O requires 498. |
| 137 | 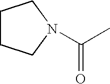 | 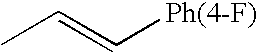 | Found 518 (MH⁺). C₃₂H₄₀FN₃O₂requires 517. |
| 138 | 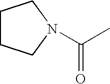 | 3-(7-aza)indolyl | Found 514 (MH⁺). C₃₁H₃₉N₅O₂requires 513. |
| 139 | 5-pyrimidinyl | 3-(3-(5-methyl)-1,2,4- | Found 537 (MH⁺). |
| | | oxadiazolyl)phenyl | C₃₂H₃₆N₆O₂ |
| | | | requires 536. |
| 140 | 5-pyrimidinyl | 5-quinolinyl(2-Me) | Found 520 (MH⁺). |
| | | | C₃₃H₃₇N₅O |
| | | | requires 519. |
| 141 | 5-pyrimidinyl | 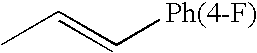 | Found 499 (MH⁺). C₃₁H₃₅FN₄O requires 498. |
| 142 | 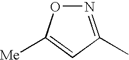 | 5-quinolinyl(2-Me) | Found 523 (MH⁺). C₃₃H₃₈N₄O₂requires 522. |
| 143 | 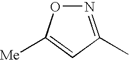 | trans-CH═CHC₆H₄(2- CN) | Found 509 (MH⁺). C₃₃H₃₈N₄O₂requires 522. |
| 144 | 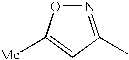 | trans-CH═CHC₆H₄(3- CN) | Found 509 (MH⁺). C₃₂H₃₆N₄O₂requires 508. |
| 145 | 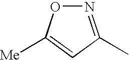 | trans-CH═CHC₆H₄(4- CN) | Found 509 (MH⁺). C₃₂H₃₆N₄O₂requires 508. |
| 146 | MeSO₂O— | 5-quinolinyl(8-F, 2-Me) | Found 554 (MH⁺). |
| | | | C₃₀H₃₆FN₃O₄S |
| | | | requires 553. |
| 147 | MeSO₂O— | —C₆H₄(3-(2-(5-Methyl)- | Found 552 (MH⁺). |
| | | oxazolyl)) | C₃₀H₃₇N₃O₅S requires |
| | | | 551. |

### Example 12

### trans-(E)-7-Cyano-3-(2-(1-(4-(3-(4-fluoro)phenylpropenoyl)amino)cyclohexyl)ethyl)-2,3,4,5-tetrahydro-1H-3-benzazepine

A mixture of trans-3-(2-(1-(4-amino)cyclohexyl)ethyl)-7-cyano-2,3,4,5-tetrahydro-1H-3-benzazepine (103 mg, 0.35 mmol), 4-fluorocinnamic acid (58 mg, 0.35 mmol), 1-(3-dimethylaminopropyl)-3-ethylcarbodiimide hydrochloride (67 mg, 0.35 mmol), and 1-hydroxybenzotriazole (20 mg, 0.15 mmol) in dichloromethane (8 ml) was shaken at room temperature for 16 h. The reaction mixture was washed with saturated sodium bicarbonate (4 ml). The resulting precipitate was collected by filtration, washed with water (2×10 ml), and dried to give the title compound (87 mg, 56%) as a colourless solid.

Mass spectrum (API⁺): Found 446 (MH⁺). C₂₈H₃₂FN₃O requires 445.

¹H NMR (DMSO-d₆) δ: 0.94-1.31 (8H, m), 1.81 (4H, m), 2.40 (5H, m), 3.04 (4H, m), 3.63 (1H, m), 6.54 (1H, d, J=16 Hz), 7.32 (4H, m), 7.59 (4H, m), 7.99 (1H, d, J=8 Hz).

### Example 13

### trans-7-Cyano-3-(2-(1-(4-(3-pyrrolo[2,3-b]pyridyl)carboxamido)cyclohexyl)ethyl)-2,3,4,5-tetrahydro-1H-3-benzazepine

A mixture of trans-3-(2-(1-(4-amino)cyclohexyl)ethyl)-7-cyano-2,3,4,5-tetrahydro-1H-3-benzazepine (103 mg, 0.35 mmol), 3-pyrrolo[2,3-b]pyridyl carboxylic acid (56 mg, 0.35 mmol), 1-(3-dimethylaminopropyl)-3-ethylcarbodiimide hydrochloride (67 mg, 0.35 mmol) and 1-hydroxybenzotriazole (20 mg, 0.15 mmol) in dichloromethane (8 ml) was shaken at room temperature for 16 h. The reaction mixture was washed with saturated aqueous sodium bicarbonate (4 ml). The resulting precipitate was collected by filtration, washed with water (2×10 ml) and dried to give the title compound (81 mg, 0.18 mmol, 53%) as a colourless solid.

Mass spectrum (API⁺): Found 442 (MH⁺). C₂₇H₃₁N₅O requires 441.

¹H NMR (DMSO-d₆) δ: 1.02 (2H, m), 1.15-1.45 (6H, m), 1.81 (4H, m), 2.50 (5H, m), 2.91 (4H, m), 3.73 (1H, m), 7.14 (1H, m), 7.32 (1H, d, J=8 Hz), 7.57 (2H, m), 7.73 (1H, d, J=8 Hz), 8.16 (1H, m), 8.25 (1H, m), 8.42 (1H, m), 12.03 (1H, br s).

### Example 14

### trans-7-Cyano-3-(2-(1-(4-(3-(3-(5-methyl)-1,2,4-oxadiazolyl)benzoyl)amino)-cyclohexyl)ethyl)-2,3,4,5-tetrahydro-1H-3-benzazepine

A mixture of trans-3-(2-(1-(4-amino)cyclohexyl)ethyl-7-cyano-2,3,4,5-tetrahydro-1H-3-benzazepine (103 mg, 0.35 mmol), 3-(3-(5-methyl)-1,2,4-oxodiazolyl)benzoic acid (71 mg, 0.35 mmol), 1-(3-dimethylaminopropyl)-3-ethylcarbodiimide hydrochloride (67 mg, 0.35 mmol) and 1-hydroxybenzotriazole (20 mg, 0.15 mmol) in dichloromethane (8 ml) was shaken at room temperature for 16 h. The reaction mixture was washed with saturated aqueous sodium bicarbonate (4 ml). The organic layer was pipetted onto a 10 g pre-packed silica column and eluted with 30-100% ethyl acetate in hexane. The fractions containing the title compound were combined and evaporated in vacuo to give the title compound (119 mg, 71%) as a colourless solid.

Mass spectrum (API⁺): Found 484. C₂₉H₃₃N₅O₂ requires 483.

¹H NMR (CDCl₃) δ: 1.08-1.35 (5H, m), 1.45 (2H, m), 1.84 (2H, m), 2.12 (2H, m), 2.50 (2H, m), 2.62 (4H, m), 2.68 (3H, s), 2.96 (4H, m), 3.95 (1H, m), 6.02 (1H, d, J=8 Hz), 7.17 (1H, d, J=8 Hz), 7.41 (2H, m), 7.57 (1H, t, J=8 Hz), 7.98 (1H, m), 8.17 (1H, m), 8.32 (1H, m).

### Example 15

### trans-(E)-7-Cyano-3-(2-(1-(4-(5-quinolinyl)carboxamido)cyclohexyl)ethyl)-2,3,4,5-tetrahydro-1H-benzazepine

A mixture of trans-3-(2-(1-(4-amino)cyclohexyl)ethyl)-7-cyano-2,3,4,5-tetrahydro-1H-benzazepine (0.10 g, 0.34 mmol), quinoline-5-carboxylic acid (0.057 g, 0.37 mmol), 1-ethyl-3-(3-dimethylaminopropyl)carbodiimide hydrochloride (0.065 g, 0.34 mmol), 1-hydroxybenzotriazole (catalytic amount) and dichloromethane (8 ml) was shaken for 16 h. Saturated sodium bicarbonate (4 ml) was then added and the mixture shaken for 0.25 h. Chromatography on the organic layer on silica eluting with a gradient of 30-100% ethyl acetate in hexane and then 0-10% methanol in ethyl acetate gave the title compound (0.130 g, 86%).

Mass spectrum (API⁺) Found 453 (MH⁺). C₂₉H₃₂N₄O requires 452.

¹H NMR (CDCl₃) δ: 1.12-1.35 (5H, m), 1.41-1.51 (2H, m), 1.83-1.89 (2H, m), 2.15-2.24 (2H, m), 2.48-2.55 (2H, m), 2.60-2.66 (4H, m), 2.91-2.99 (4H, m), 3.97-4.13 (1H, m), 5.86 (1H, d, J=8 Hz), 7.18 (1H, d, J=8 Hz), 7.37-7.49 (3H, m), 7.63-7.70 (2H, m), 8.15-8.20 (1H, m), 8.71-8.76 (1H, m), 8.94-8.96 (1H, m).

### Example 16

### trans-(E)-7-Cyano-3-(2-(1-(4-(3-(3-acetylamino)phenylpropenoyl)amino)cyclohexyl)ethyl)-2,3,4,5-tetrahydro-1H-3-benzazepine

A mixture of trans-3-(2-(1-(4-amino)cyclohexyl)ethyl)-7-cyano-2,3,4,5-tetrahydro-1H-3-benzazepine (0.10 g, 0.34 mmol), 3-acetamido cinnamic acid (0.076 g, 0.42 mmol), 1-ethyl-3-(3-dimethylaminopropyl)carbodiimide hydrochloride (0.071 g, 0.42 mmol), 1-hydroxybenzotriazole (catalytic amount) and dichloromethane (8 ml) was shaken for 16 h. Saturated sodium bicarbonate (4 ml) was then added and the mixture shaken for 0.25 h. The precipitated solid was filtered off and washed with water then diethyl ether, and dried to give the title compound (0.12 g, 74%) as a colourless solid.

Mass spectrum (API⁺): Found 485 (MH⁺). C₃₀H₃₆N₄O₂ requires 484.

¹H NMR (CDCl₃+CD₃OD) δ: 1.02-1.35 (5H, m), 1.35-1.50 (2H, m), 1.77-1.82 (2H, m), 2.00-2.04 (2H, m), 2.17 (3H, s), 2.47-2.55 (6H, m), 2.93-2.99 (4H, m), 3.70-3.85 (1H, m), 6.41 (1H, d, J=15 Hz), 7.17-7.30 (4H, m), 7.38-7.43 (3H, m), 7.50 (1H, d, J=16 Hz), 7.80 (1H, s).

### Example 17

### trans-7-Cyano-3-(2-(1-(4-(6-(3,4-dihydro-3-oxo)-2H-benzoxazinyl)carboxamido)-cyclohexyl)ethyl)-2,3,4,5-tetrahydro-1H-benzazepine

A mixture of trans-3-(2-(1-(4-amino)cyclohexyl)ethyl)-7-cyano-2,3,4,5-tetrahydro-1H-3-benzazepine (0.10 g, 0.34 mmol), 2,3-dihydro-3-oxo-4H-benzoxazine-6-carboxylic acid (0.072 g, 0.42 mmol), 1-ethyl-3-(3-dimethylaminopropyl)carbodiimide hydrochloride (0.071 g, 0.42 mmol), 1-hydroxybenzotriazole (catalytic amount) and dichloromethane (8 ml) was shaken for 16 h. Saturated sodium bicarbonate (4 ml) was then added and the mixture shaken for 0.25 h. The precipitated solid was filtered off and washed with water then diethyl ether, and dried to give the title compound (0.16 g, 100%) as a colourless solid.

Mass spectrum (API⁺): Found 473 (MH⁺). C₂₈H₃₂N₄O₃ requires 472.

¹H NMR (DMSO-d₆) δ: 0.95-1.50 (7H, m), 1.75-1.95 (4H, m), 2.40-2.65 (6H, m), 2.93-3.05 (4H, m), 3.69-3.82 (1H, m), 4.67 (2H, s), 7.02 (1H, d, J=8 Hz), 7.39 (1H, d, J=8 Hz), 7.46-7.50 (2H, m), 7.65 (2H, m), 8.13 (1H, d, J=8 Hz).

### Example 18

### trans-(E)-7-Cyano-3-(2-(1-(4-(3-(6-(1,2-dihydro-2-oxo)quinolinyl)propenoyl)amino)-cyclohexyl)ethyl)-2,3,4,5-tetrahydro-1H-3-benzazepine

A mixture of trans-3-(2-(1-(4-amino)cyclohexyl)ethyl)-7-cyano-2,3,4,5-tetrahydro-1H-3-benzazepine (288 mg, 0.97 mmol), trans-3-(6-(1,2-dihydro-2-oxo)quinolinyl)-propenoic acid (250 mg, 1.16 mmol), 1-ethyl-3-(3-dimethylaminopropyl)carbodiimide hydrochloride (204 mg, 1.07 mmol), 1-hydroxybenzotriazole (catalytic amount) and DMF (20 ml) was shaken for 18 h. Saturated sodium bicarbonate (8 ml) was then added and the mixture shaken for 0.25 h. The resulting precipitate was filtered and dried in vacuo to give the title compound (370 mg, 77%) as a colourless solid.

Found: 495 (MH⁺). C₃₁H₃₄N₄O₂ requires 494.

¹H NMR (DMSO-d₆) δ: 0.94-1.05 (2H, m), 1.10-1.30 (3H, m), 1.30-1.40 (2H, m), 1.74-1.80 (2H, m), 1.80-1.88 (2H, m), 2.44 (2H, t, J=7.5 Hz), 2.45-2.55 (4H, m), 2.85-2.95 (4H, m), 3.55-3.65 (1H, m), 6.50-6.60 (2H, m), 7.28-7.35 (2H, m), 7.40 (1H, d, J=16 Hz), 7.55-7.60 (2H, m), 7.68-7.72 (1H, m), 7.81 (1H, s), 7.93 (1H, d, J=16 Hz), 7.94-8.00 (2H, m).

### Example 19

### trans-(E)-7-Cyano-3-(2-(1-(4-(3-(2-fluoro-4-acetylamino)phenylpropenoyl)amino)-cyclohexyl)ethyl)-2,3,4,5-tetrahydro-1H-3-benzazepine

A mixture of trans-3-(2-(1-(4-amino)cyclohexyl)ethyl)-7-cyano-2,3,4,5-tetrahydro-1H-3-benzazepine (150 mg, 0.51 mmol), (E)-(2-fluoro-4-acetylamino)phenylpropenoic acid (113 mg, 0.51 mmol), EDC. hydrochloride (97 mg, 0.51 mmol) and 1-hydroxybenzotriazole in dichloromethane (10 ml) was shaken at room temperature for 16 h. The reaction mixture was washed with saturated aqueous sodium bicarbonate (4 ml) and the precipitate collected by filtration and then re-suspended in water and filtered before drying in vacuo to give the title compound as an off white solid (200 mg, 79%).

Mass spectrum (API⁺): Found 503. C₃₀H₃₅FN₄O₂ requires 502.

¹H NMR δ (DMSO-d₆+TFA): 0.95-1.34 (5H, m), 1.61 (2H, m), 1.82 (4H, m), 2.07 (3H, s), 3.06 (2H, m), 3.18 (6H, m), 3.68 (3H, m), 6.59 (1H, d, J=16 Hz), 7.34 (2H, m), 7.39-7.63 (3H, m), 7.72 (2H, m), 8.03 (1H, d, J=8 Hz), 9.74 (1H, br s), 10.29 (1H, s).

### Example 20

### trans-(E)-7-Cyano-3-(2-(1-(4-(3-(8-(1,2-dihydro-2-oxo)quinolinyl)propenoyl)amino)cyclohexyl)ethyl)-2,3,4,5-tetrahydro-1H-3-benzazepine

A mixture of trans-3-(2-(1-(4-amino)cyclohexyl)ethyl-7-cyano-2,3,4,5-tetrahydro-1H-3-benzazepine (0.25 g, 0.84 mmol), (E)-3-(8-(1,2-dihydro-2-oxo)quinolinyl)propenoic acid (0.27 g, 1.2 mmol), EDC. hydrochloride (0.3 g, 1.5 mmol) and 1-hydroxybenzotriazole (50 mg) in DMF (10 ml) was allowed to stir at 80° C. for 4 h, then poured into water (500 ml). The precipitate was collected by filtration and then re-suspended in aqueous sodium bicarbonate solution. Resulting solid was collected by filtration, then washed with water and diethyl ether, then was dried in vacuo to give the title compound (0.42 g, 95%) as a yellow solid.

Found: 495 (MH⁺). C₃₁H₃₄N₄O₂ requires 494.

δ (DMSO-d₆+TFA): 1.00-1.15 (2H, m), 1.15-1.30 (3H, m), 1.50-1.70 (2H, m), 1.70-1.85 (2H, m), 1.85-1.95 (2H, m), 2.95-3.30 (8H, m), 3.60-3.80 (3H, m), 6.45-6.60 (2H, m), 7.23 (1H, t, J=8 Hz), 7.46 (1H, d, J=8 Hz), 7.60-7.80 (4H, m), 7.94 (1H, d, J=10 Hz), 7.95-8.10 (3H, m), 9.70 (1H, br s).

### Example 21

### trans-7-Cyano-3-(2-(1-(4-(5-(8-fluoro)quinolinyl)carboxamido)cyclohexyl)ethyl)-2,3,4,5-tetrahydro-1H-3-benzazepine

A mixture of trans-3-(2-(1-(4-amino)cyclohexyl)ethyl)-7-cyano-2,3,4,5-tetrahydro-1H-3-benzazepine (0.162 g, 0.545 mmol), 8-fluoroquinoline-5-carboxylic acid (0.115 g, 0.6 mmol), 1-(3-dimethylaminopropyl)-3-ethylcarbodiimide hydrochloride (0.115 g, 0.6 mmol) and 1-hydroxybenzotriazole hydrate (0.01 g, 0.065 mmol) in dichloromethane (7 ml) was shaken for 18 h. Saturated aqueous sodium hydrogen carbonate (6 ml) was added and shaking continued for 0.5 h. The organic layer was separated and pipetted onto a column of silica (10 g). Elution with 30-100% ethyl acetate-hexane gradient then 1-10% methanol-ethyl acetate gradient yielded the title compound as a colourless solid (0.22 g, 85%).

Mass spectrum (API⁺): Found 471 (MH⁺). C₂₉H₃₁FN₄O requires 470.

¹H NMR (CDCl₃) δ: 1.05-1.40 (5H, m), 1.45 (2H, m), 1.85 (2H, m), 2.20 (2H, m), 2.55 (2H, m), 2.63 (4H, m), 2.96 (4H, m), 4.00 (1H, m), 5.86 (1H, d, J=8 Hz), 7.17 (1H, d, J=8 Hz), 7.30-7.45 (3H, m), 7.54 (1H, m), 7.62 (1H, m), 8.80 (1H, d, J=8 Hz), 9.01 (1H, m).

### Example 24

### trans-3-(2-(1-(4-(5-(2-Methyl)quinolinyl)carboxamido)cyclohexyl)ethyl)-7-methylsulphonyl-2,3,4,5-tetrahydro-1H-3-benzazepine

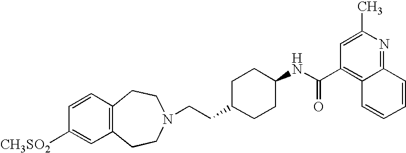

A mixture of trans-3-(2-(1-(4-amino)cyclohexyl)ethyl)-7-methylsulphonyl-2,3,4,5-tetrahydro-1H-3-benzazepine (100 mg, 0.29 mmol), 2-methyl-quinoline-5-carboxylic acid (64 mg, 0.34 mmol), 1-ethyl-3-(3-dimethylaminopropyl)carbodiimide hydrochloride (59 mg, 0.31 mmol) and 1-hydroxybenzotriazole (cat. amt.) in dichloromethane (10 ml) was shaken at room temperature for 18 h. A saturated solution of sodium bicarbonate (4 ml) was then added and the mixture shaken for 0.25 h. The organic layer was then applied directly to a silica column eluted with a gradient of 30-100% ethyl acetate in hexane and then 0-10% methanol in ethyl acetate to give the title compound (95 mg, 66%) as a colourless solid.

¹H NMR δ (CDCl₃) 1.15-1.30 (5H, m), 1.44-1.50 (2H, m), 1.82-1.88 (2H, m), 2.15-2.20 (2H, m), 2.53 (2H, t, J=7.6 Hz), 2.62-2.68 (4H, m), 2.75 (3H, s), 2.98-3.02 (4H, m), 3.04 (3H, s), 3.95-4.05 (1H, m), 5.84 (1H, d, J=8.2 Hz), 7.28 (1H, d, J=7.9 Hz), 7.35 (1H, d, J=8.8 Hz), 7.56-7.70 (4H, m), 8.08 (1H, d), 8.62 (1H, d).

Mass spectrum: API⁺ 520 (MH⁺): C₃₀H₃₇N₃SO₃ requires 519.

### Example 25

### trans-3-(2-(1-(4-(3-(3-(5-Methyl)-1,2,4-oxadiazolyl)benzoyl)amino)cyclohexyl)ethyl)-7-methylsulphonyl-2,3,4,5-tetrahydro-1H-3-benzazepine

A mixture of trans-3-(2-(1-(4-amino)cyclohexyl)-7-methylsulphonyl-2,3,4,5-tetrahydro-1H-3-benzazepine (100 mg, 0.29 mmol), 3-(3-(5-methyl)-1,2,4-oxadiazolyl)-benzoic acid (69 mg, 0.34 mmol), 1-ethyl-3-(3-dimethylaminopropyl)carbodiimide hydrochloride (59 mg, 0.31 mmol) and 1-hydroxybenzotriazole (cat. amt.) in dichloromethane (10 ml) was shaken at room temperature for 18 h. A saturated solution of sodium bicarbonate (4 ml) was then added and the mixture shaken for 0.25 h. The organic layer was then applied directly to a silica column eluted with a gradient of 30-100% ethyl acetate in hexane and then 0-10% methanol in ethyl acetate to give the title compound (103 mg, 69%) as a colourless solid.

¹H NMR δ (CDCl₃): 1.08-1.30 (5H, m), 1.40-1.46 (2H, m), 1.80-1.85 (2H, m), 2.08-2.15 (2H, m), 2.52 (2H, t, J=7.8), 2.60-2.65 (4H, m), 2.68 (3H, s), 2.98-3.02 (4H, m), 3.05 (3H, s), 3.90-4.00 (1H, m), 6.01 (1H, d, J=8.0 Hz), 7.28 (1H, d, J=7.28 Hz), 7.57 (1H, t, J=7.8 Hz), 7.65-7.70 (2H, m), 8.0 (1H, d), 8.19 (1H, d, J=7.7 Hz), 8.32 (1H, s).

Mass spectrum: API⁺ 537 (MH⁺): C₂₉H₃₆N₄SO₄ requires 536.

### Example 26

### trans-7-(5-(3-Methyl)-1,2,4-oxadiazolyl)-3-(2-(1-(4-(3-pyrrolo[2,3-b]pyridyl)carboxamido)cyclohexyl)ethyl)-2,3,4,5-tetrahydro-1H-3-benzazepine, hydrochloride

A mixture of trans-3-(2-(1-(4-amino)cyclohexyl)-7-(5-(3-methyl)-1,2,4-oxadiazolyl)-2,3,4,5-tetrahydro-1H-3-benzazepine (21.0 g, 59.3 mmol), pyrrolo[2,3-b]pyridyl-3-carboxylic acid (10.57 g, 65.2 mmol), EDC hydrochloride (12.46 g, 64.4 mmol) and HOBT (0.5 g) in CH₂Cl₂ (630 ml) and DMF (84 ml) was stirred at ambient temperature for 16 h. Saturated aqueous sodium bicarbonate (350 ml) was added and the mixture stirred for 0.25 h. The precipitate was collected by filtration, washed in turn with water and diethyl ether and dried in vacuo to give the free base of the title compound (18.0 g, 61%).

Mass spectrum (API⁺): Found 499 (MH⁺). C₂₉H₃₄N₆O₂ requires 498.

NMR (DMSO-d₆) δ: 0.90-1.10 (2H, m), 1.10-1.40 (5H, m), 1.70-1.90 (4H, m), 2.40-2.70 (6H, m), 2.96 (3H, s), 3.31 (4H, m), 3.89 (1H, m), 7.15 (1H, m), 7.36 (1H, d, J=8 Hz), 7.71 (1H, d, J=8 Hz), 7.75-7.85 (2H, m), 8.12 (1H, s), 8.20 (1H, s), 8.35 (1H, d, J=8 Hz), 12.02 (1H, br s).

To a suspension of the above free base (18.0 g, 36 mmol) in 10% methanol-dichloromethane (500 ml) was added a 1M solution of HCl in diethyl ether (37.08 ml). The resulting solution was evaporated in vacuo and the residue crystallised from methanol to give the title compound as a colourless solid (12.5 g, m.p. 275-276° C.).

### Example 27

### trans-(E)-7-(3-(5-Methyl)-1,2,4-oxadiazolyl)-3-(2-(1-(4-(3-(4-fluoro)phenylpropenoyl)amino)cyclohexyl)ethyl)-2,3,4,5-tetrahydro-1H-3-benzazepine

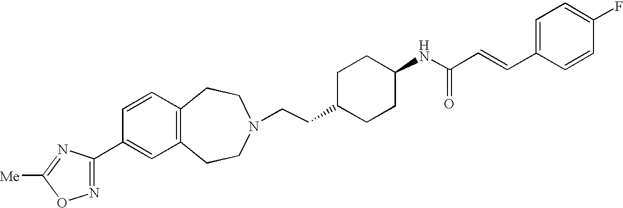

Alternative Name: (2E)-3-(4-fluorophenyl)-N-[trans-4-[2-[2,3,4,5-tetrahydro-7-(5-methyl-1,2,4-oxadiazol-3-yl)-1H-3-benzazepin-3-yl]ethyl]cyclohexyl]-2-propenamide

A mixture of trans-3-(2-(1-(4-amino)cyclohexyl)ethyl)-7-(3-(5-methyl)-1,2,4-oxadiazolyl)-2,3,4,5-tetrahydro-1H-3-benzazepine (0.1 g, 0.28 mmol), (E)-4-fluorocinnamic acid (0.046 g, 0.28 mmol), EDC hydrochloride (0.06 g, 0.31 mmol) and HOBT (0.015 g) in dichloromethane (8 ml) was stirred at ambient temperature for 64 h, then was washed with saturated aqueous sodium bicarbonate (4 ml). The organic phase was purified by silica gel chromatography eluting with 0-10% methanol in ethyl acetate, to give the title compound (0.12 g, 85%) as a colourless solid.

Mass spectrum (API⁺): Found 503 (MH⁺). C₃₀H₃₅FN₄O₂ requires 502.

NMR (CDCl₃) δ: 1.10-1.80 (4H, m), 1.25 (1H, m), 1.44 (2H, m), 1.78 (2H, m), 2.06 (2H, m), 2.50 (2H, m), 2.60-2.70 (7H, m), 2.90-3.00 (4H, m), 3.85 (1H, m), 5.39 (1H, d, J=8 Hz), 6.26 (1H, d, J=16 Hz), 7.05 (2H, t, J=8 Hz), 7.20 (1H, d, J=8 Hz), 7.47 (2H, m), 7.57 (1H, d, J=16 Hz), 7.80-7.90 (2H, m).

A mixture of trans-3-(2-(1-(4-amino)cyclohexyl)ethyl)-7-(3-(5-methyl)-1,2,4-oxadiazolyl)-2,3,4,5-tetrahydro-1H-3-benzazepine (16.0 g, 0.045 mol), (E)-4-fluorocinnamic acid (7.5 g, 0.045 mol), EDC hydrochloride (9.53 g, 0.050 mol), and HOBT (0.78 g, 0.006 mol) in dichloromethane (0.78 L) was stirred under argon at ambient temperature for 111 h. Saturated aqueous sodium bicarbonate (1 L) was added and after stirring for 0.25 h, the mixture was filtered and the solid washed with saturated aqueous sodium bicarbonate (2×0.25 L), water (3×0.25 L), diethyl ether (3×0.25 L) and dried in vacuo to afford the title compound (18.4 g, 81%) as a colourless solid.

The filtrate was separated and the aqueous layer extracted with dichloromethane (2×0.3 L). The combined extracts were dried and evaporated in vacuo to afford a pale yellow solid (4.5 g). Sequential trituration with dichloromethane (0.08 L), saturated aqueous sodium bicarbonate (1×0.5 L; 2×0.2 L), water (3×0.2 L), and diethyl ether (3×0.2 L) followed by drying in vacuo afforded the title compound (2.8 g, 12%) as a colourless solid.

Both batches had spectroscopic data identical to that described above. To a solution of the free base obtained above (21.2 g, 0.042 mol) in dichloromethane (0.55 L) and methanol (0.1 L) was added 1M hydrogen chloride in diethyl ether (0.051 L, 0.05 mol). The resulting solution was evaporated in vacuo and the residue crystallised from methanol to afford (2E)-3-(4-fluorophenyl)-N-[trans-4-[2-[2,3,4,5-tetrahydro-7-(5-methyl-1,2,4-oxadiazol-3-yl)-1H-3-benzazepin-3-yl]ethyl]cyclohexyl]-2-propenamide monohydrochloride (19.8 g, 91%) as a colourless solid m.p. 259-261° C.

NMR (D₆-DMSO) δ: 1.00-1.09 (2H, m), 1.15-1.28 (3H, m), 1.60-1.70 (2H, m), 1.70-1.80 (2H, m), 1.80-1.90 (2H, m), 2.66 (3H, s), 2.95-3.25 (6H, m), 3.35-3.50 (2H, m), 3.55-3.75 (3H, m), 6.55 (1H, d, J=16 Hz), 7.22-7.27 (2H, m), 7.39 (1H, d, J=16 Hz), 7.40-7.45 (1H, m), 7.55-7.64 (2H, m), 7.80-7.85 (1H, m), 7.87 (1H, s), 7.95-8.05 (1H, m), 10.60 (1H, br s).

### Example 28

### trans-(E)-7-(5-(3-Methyl)-1,2,4-oxadiazolyl)-3-(2-(1-(4-(3-(4-fluoro)phenylpropenoyl)amino)cyclohexyl)ethyl)-2,3,4,5-tetrahydro-1H-3-benzazepine

A mixture of trans-3-(2-(1-(4-amino)cyclohexyl)ethyl)-7-(5-(3-methyl)-1,2,4-oxadiazolyl)-2,3,4,5-tetrahydro-1H-3-benzazepine (0.1 g, 0.28 mmol), (E)-4-fluorocinnamic acid (0.046 g, 0.28 mmol) EDC hydrochloride (0.06 g, 0.31 mmol) and HOBT (0.015 g) in dichloromethane (8 ml) was stirred at ambient temperature for 64 h, then was washed with saturated aqueous sodium bicarbonate (4 ml). The organic phase was purified by silica gel chromatography eluting with 0-10% methanol in ethyl acetate to give the title compound (0.12 g, 85%) as a colourless solid.

Mass spectrum (API⁺): Found 503 (MH⁺). C₃₀H₃₅FN₄O₂ requires 502.

NMR (CDCl₃) δ: 1.10-1.30 (5H, m), 1.40-1.47 (2H, m), 1.78-1.82 (2H, m), 2.00-2.10 (2H, m), 2.46 (3H, s), 2.47-2.52 (2H, m), 2.60-2.70 (4H, m), 2.95-3.05 (4H, m), 3.86 (1H, m), 5.38 (1H, d, J=8 Hz), 6.26 (1H, d, J=16 Hz), 7.05 (2H, t, J=8 Hz), 7.24 (1H, d, J=8 Hz), 7.47 (2H, dd, J=5, 8 Hz), 7.57 (1H, d, J=16 Hz) 7.80-7.90 (2H, m).

### Example 29

### trans-(E)-7-(5-(3-Methyl)isoxazolyl)-3-(2-(1-(4-(3-(4-fluoro)phenylpropenoyl)amino)cyclohexyl)ethyl)-2,3,4,5-tetrahydro-1H-3-benzazepine

A mixture of trans-3-(2-(1-(4-amino)cyclohexyl)ethyl-7-(5-(3-methyl)isoxazolyl)-2,3,4,5-tetrahydro-1H-3-benzazepine (0.1 g, 0.28 mmol), 4-fluorophenylacetic acid (0.044 g, 0.28 mmol), EDC hydrochloride (0.065 g, 0.31 mmol) and HOBT (0.02 g) in CH₂Cl₂ (8 ml) was stirred at ambient temperature for 16 h, then was washed with saturated sodium bicarbonate (4 ml). The organic phase was purified by silica gel chromatography eluting with 0-10% methanol in ethyl acetate to give the title compound (0.1 g, 73%).

Mass spectrum (API⁺): Found 490 (MH⁺). C₃₀H₃₆FN₃O₂ requires 489.

¹H NMR δ (CDCl₃): 0.90-1.10 (4H, m), 1.10-1.20 (1H, m), 1.30-1.40 (2H, m), 1.70-1.80 (2H, m), 1.85-1.95 (2H, m), 2.34 (3H, s), 2.40-2.50 (2H, m), 2.55-2.70 (4H, m), 2.90-3.00 (4H, m), 3.50 (2H, s), 3.65-3.80 (1H, m), 5.12 (1H, d, J=8 Hz), 6.30 (1H, s), 7.03 (2H, t, J=8 Hz), 7.15 (1H, d, J=8 Hz), 7.19-7.25 (2H, m), 7.45-7.52 (2H, m).

### Example 148

### trans-7-(5-(3-Methyl)-1,2,4-oxadiazolyl)-3-(2-(1-(4-(4-fluoro)phenylacetamido)cyclohexyl)ethyl)-2,3,4,5-tetrahydro-1H-3-benzazepine

A mixture of trans-3-(2-(1-(4-amino)cyclohexyl)ethyl)-7-(5-(3-methyl)-1,2,4-oxadiazolyl)-2,3,4,5-tetrahydro-1H-3-benzazepine (0.1 g, 0.28 mmol), (4-fluoro)phenylacetic acid (0.044 g, 0.28 mmol), EDC hydrochloride (0.054 g, 0.28 mmol) and HOBT (0.015 g) in dichloromethane (5 ml) was shaken at ambient temperature for 16 h, and saturated aqueous sodium bicarbonate (4 ml) added. The organic phase was purified by silica gel chromatography eluting with 30-100% ethyl acetate in hexane, then 0-10% methanol in ethyl acetate gradient elution to give the title compound (0.095 g, 70%) as a colourless solid.

Mass spectrum (API⁺): Found 491 (MH⁺). C₂₉H₃₅FN₄O₂ requires 490.

¹H NMR δ (CDCl₃): 0.90-1.30 (5H, m), 1.35-1.50 (2H, m), 1.70-1.80 (2H, m), 1.85-1.95 (2H, m), 2.46 (3H, s), 2.40-2.50 (2H, m), 2.55-2.65 (4H, m), 2.95-3.00 (4H, m), 3.50 (2H, s), 3.60-3.80 (1H, m), 5.13 (1H, d, J=8 Hz), 6.95-7.08 (2H, m), 7.15-7.30 (3H, m), 7.80-7.90 (2H, m).

### Example 149

### trans-3-(2-(1-(4-(5-(2-Methyl)quinolinyl)carboxamide)cyclohexyl)ethyl)-7-methanesulphonyloxy-2,3,4,5-tetrahydro-1H-3-benzazepine

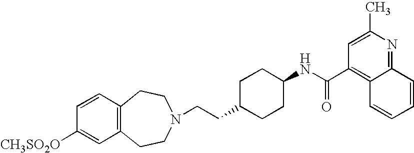

A mixture of trans-3-(2-(1-(4-amino)cyclohexyl)ethyl)-7-methanesulphonyloxy-2,3,4,5-tetrahydro-1H-3-benzazepine (150 mg, 0.41 mmol), 2-methyl-quinoline-5-carboxylic acid (92 mg, 0.49 mmol), 1-ethyl-3-(3-dimethylaminopropyl)carbodiimide hydrochloride (86 mg, 0.45 mmol) and 1-hydroxybenzotriazole (cat. amt.) in dichloromethane (10 ml) was shaken at room temperature for 18 h. A saturated solution of sodium bicarbonate (4 ml) was added and the mixture shaken for 0.25 h. The organic layer was then applied directly to a silica column eluted with a gradient of 30-100% ethyl acetate in hexane and then 0-10% methanol in ethyl acetate to give the title compound (161 mg, 74%) as a colourless solid.

¹H NMR (CDCl₃) δ: 1.15-1.30 (5H, m), 1.45-1.50 (2H, m), 1.82-1.90 (2H, m), 2.15-2.20 (2H, m), 2.50-2.55 (2H, m), 2.60-2.68 (4H, m), 2.75 (3H, s), 2.90-2.95 (4H, m), 3.13 (3H, s), 3.95-4.05 (1H, m), 5.82 (1H, d, J=8.2 Hz), 7.00-7.03 (2H, m), 7.12 (1H, d, J=7.8 Hz), 7.35 (1H, d, J=8.8 Hz), 7.55-7.70 (2H, m), 8.08 (1H, d, J=8.3 Hz), 8.61 (1H, d).

Mass Spectrum (AP⁺): Found 536 (MH⁺). C₃₀H₃₇N₃SO₄ requires 535.

### BIOLOGICAL EXAMPLES

The inhibitory effects of compounds at the M₃ mAChR of the present invention are determined by the following in vitro and in vivo functional assays:

Analysis of Inhibition of Receptor Activation by Calcium Mobilization:

Stimulation of mAChRs expressed on CHO cells were analyzed by monitoring receptor-activated calcium mobilization as previously described (Sarau, H. M., R. S. Ames, J. Chambers, C. Ellis, N. Elshourbagy, J. J. Foley, D. B. Schmidt, R. M. Muccitelli, O. Jenkins, P. R. Murdock, N. C. Herrity, W. Halsey, G. Sathe, A. I. Muir, P. Nuthulaganti, G. M. Dytko, P. T. Buckley, S. Wilson, D. J. Bergsma, and D. W. Hay. 1999. Identification, molecular cloning, expression, and characterization of a cysteinyl leukotriene receptor. Mol Pharmacol 56:657-663). CHO cells stably expressing M₃ mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and incubated 1 hr at 37° C. The dye-containing media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂ PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵ M final in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels (Sullivan, E., E. M. Tucker, and I. L. Dale. 1999. Measurement of [Ca2+] using the Fluorometric Imaging Plate Reader (FLIPR). Methods Mol Biol 114:125-133). The emitted fluorescence from all 96 wells is measured simultaneously using a cooled CCD camera. Data points are collected every second. This data was then plotting and analyzed using GraphPad PRISM software.

Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine (Hamelmann, E., J. SCHWARZE, K. TAKEDA, A. OSHIBA, G. á. LARSEN, C. á. IRVIN, and E. á. GELFAND. 1997. Noninvasive Measurement of Airway Responsiveness in Allergic Mice Using Barometric Plethysmography. Am. J. Respir. Crit. Care Med. 156:766-775). Mice were pretreated with 50 μl of compound (0.003-10 μg/mouse) in 50 μl of vehicle (10% DMSO) intranasally, i.v., i.p. or p.o, and were then placed in the plethysmography chamber. Once in the chamber, the mice were allowed to equilibrate for 10 min before taking a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis; gastrointestinal-tract disorders such as irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakisuria, neurogenic bladder, nocturnal enuresis, psychosomatic bladder, incontinence associated with bladder spasms or chronic cystitis, urinary urgency or pollakiuria, and motion sickness.

Methods of administering the present compounds will be readily apparent to the skilled artisan. Dry powder compositions for topical delivery to the lung by inhalation may, for example, be presented in capsules and cartridges of for example gelatine, or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred. Each capsule or cartridge may generally contain between 20 μg-10 mg of the compound of formula (I) optionally in combination with another therapeutically active ingredient. Alternatively, the compound of the invention may be presented without excipients.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective. To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognised as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

A preferable means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomization of the spray is ensured as it will not release the formulation until the threshold pressure for effectively atomizing the spray has been achieved. Typically, the VP7 model may be used with a bottle capable of holding 10-50 ml of a formulation. Each spray will typically deliver 50-100 μl of such a formulation, therefore, the VP7 model is capable of providing at least 100 metered doses.

### EXAMPLES OF NASAL FORMULATIONS

### Example 1

### Nasal Formulation Containing Active

A formulation for intranasal delivery was prepared with ingredients as follows:

| | |
|---|---|
| | to 100% |
| Active | 0.1% w/w |
| Polysorbate 80 | 0.025% w/w |
| Avicel RC591 | 1.5% w/w |
| Dextrose | 5.0% w/w |
| BKC | 0.015% w/w |
| EDTA | 0.015% w/w |
| water | to 100% |

in a total amount suitable for 120 actuations and the formulation was filled into a bottle fitted with a metering valve adapted to dispense 50 or 100 μl per actuation. The device was fitted into a nasal actuator (Valois).

### Example 2

### Nasal Formulation Containing Active

A formulation for intranasal delivery was prepared with ingredients as follows:

| | |
|---|---|
| Active | 0.005% w/w |
| Tyloxapol | 2% w/w |
| dextrose | 5% w/w |
| BKC | 0.015% w/w |
| EDTA | 0.015% w/w |
| water | to 100% |

in a total amount suitable for 120 actuations and the formulation was filled into a bottle (plastic or glass) fitted with a metering valve adapted to dispense 50 or 100 μl per actuation

The device was fitted into a nasal actuator (Valois, e.g. VP3, VP7 or VP7D)

### Example 3

### Nasal Formulation Containing Active

A formulation for intranasal delivery was prepared with ingredients as follows:

| | |
|---|---|
| active | 0.05% w/w |
| Triton X-100 | 5% w/w |
| Dextrose | 4% w/w |
| BKC | 0.015% w/w |
| EDTA | 0.015% w/w |

water to 100% in a total amount suitable for 120 actuations and the formulation was filled into a bottle fitted with a metering valve adapted to dispense 50 or 100 μl per actuation.

### Example 4

### Nasal Formulation Containing Active

A formulation for intranasal delivery was prepared with ingredients as follows:

| | |
|---|---|
| active | 0.05% w/w |
| Tyloxapol | 5% w/w |
| dextrose | 5% w/w |
| BKC | 0.015% w/w |
| EDTA | 0.015% w/w |
| water | to 100% |

in a total amount suitable for 120 actuations and the formulation was filled into a bottle fitted with a metering valve adapted to dispense 50 or 100 μl per actuation The device was fitted into a nasal actuator (Valois).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore the Examples herein are to be construed as merely illustrative and not a limitation of the scope of the present invention in any way. The embodiments of the invention in which an exclusive property or privilege is claimed are defined as follows.

The patents and patent applications described in this application are herein incorporated by reference.

What is claimed is:

1. A compound according to Formula I herein below: 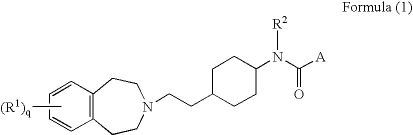
wherein:
R¹ is selected from the group consisting of a hydrogen or halogen atom; a hydroxy, cyano, nitro, trifluoromethyl, trifluoromethoxy, trifluoromethanesulfonyloxy, pentafluoroethyl, C₁₋₄alkyl, C₁₋₄alkoxy, arylC₁₋₄alkoxy, C₁₋₄alkylthio, C₁₋₄alkoxyC₁₋₄alkyl, C₃₋₆cycloalkylC₁₋₄alkoxy, C₁₋₄alkoxycarbonyl, C₁₋₄alkylsulfonyl, C₁₋₄alkylsulfonyloxy, C₁₋₄alkylsulfonylC₁₋₄alkyl, arylsulfonyl, arylsulfonyloxy, arylsulfonylC₁₋₄alkyl, C₁₋₄alkylsulfonamido, C₁₋₄alkylamido, C₁₋₄alkylsulfonamidoC₁₋₄alkyl, C₁₋₄alkylamidoC₁₋₄alkyl, arylsulfonamido, arylcarboxamido, arylsulfonamidoC₁₋₄alkyl or a arylcarboxamidoC₁₋₄alkyl group; a group R³OCO(CH₂)ₚ, R³CON(R⁴)(CH₂)ₚ, R³R⁴NCO(CH₂)ₚ and R³R⁴NSO₂(CH₂)ₚ, where each of R³ and R⁴ is, independently, selected from a group consisting of a hydrogen atom and a C₁₋₄alkyl group or R³R⁴ forms part of a C₃₋₆azacyloalkane or C₃₋₆(2-oxo)azacycloalkane ring and p represents zero or an integer from 1 to 4; or a group Ar³-Z, wherein Ar³ represents an optionally substituted phenyl ring or an optionally substituted 5- or 6-membered aromatic heterocyclic ring and Z represents a bond, O, S, or CH₂;
R² is selected from a group consisting of a hydrogen atom or a C₁₋₄alkyl group;
q is 1 or 2;
A is selected from the group consisting of a group of the formula (a), (b) (c) and (d): 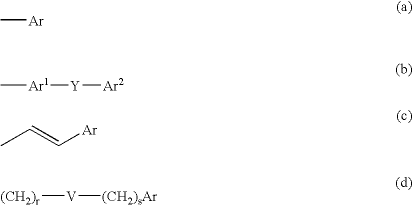
wherein
Ar is selected from a group consisting of an optionally substituted phenyl ring or an optionally substituted 5- or 6-membered aromatic heterocyclic ring; or an optionally substituted bicyclic ring system;
Ar¹ and Ar² each independently represent an optionally substituted phenyl ring or an optionally substituted 5- or 6-membered aromatic heterocyclic ring; and
Y is selected from a group consisting of a bond, —NHCO—, —CONH—, —CH₂—, or —(CH₂)ₘY¹(CH₂)ₙ—, wherein Y¹ represents O, S, SO₂, or CO and m and n each represent zero or 1 such that the sum of m+n is zero or 1; providing that when A represents a group of formula (a), any substituent present in Ar ortho to the carboxamide moiety is necessarily a hydrogen or a methoxy group;
r and s are, independently, selected from a group consisting of an integer from zero to 3 such that the sum of r and s is equal to an integer from 1 to 4; and
V is selected from a group consisting of a bond, O or S; and salts thereof.

2. A compound according to claim 1 consisting of the group selected from:
trans-3-(2-(1-(4-(4-Quinolinyl)carboxamido)cyclohexyl)ethyl)-2,3,4,5-tetrahydro-1H-3-benzazepine;
trans-(E)-3-(2-(1-(4-(3-(3-Methylsulfonyl)phenylpropenoyl)amino)cyclohexyl)ethyl)-2,3,4,5-tetrahydro-1H-3-benzazepine;
trans-(E)-3-(2-(1-(4-(3-(4-Fluoro)phenylpropenoyl)amino)cyclohexyl)ethyl)-2,3,4,5-tetrahydro-1H-3-benzazepine;
trans-3-(2-(1-(4-(2-Indolyl)carboxamido)cyclohexyl)ethyl)-2,3,4,5-tetrahydro-1H-3-benzazepine;
trans-3-(2-(1-(4-(3-(3-Pyridyl)phenyl)carboxamido)cyclohexyl)ethyl)-2,3,4,5-tetrahydro-1H-3-benzazepine;
trans-3-(2-(1-(4-Phenylacetamido)cyclohexyl)ethyl)-2,3,4,5-tetrahydro-1H-3-benzazepine;
trans-3-(2-(1-(4-(3-Indolyl)acetamido)cyclohexyl)ethyl)-2,3,4,5-tetrahydro-1H-3-benzazepine;
trans-3-(2-(1-(4-(4-Quinolinyl)acetamido)cyclohexyl)ethyl)-2,3,4,5-tetrahydro-1H-3-benzazepine;
trans-(E)-3-(2-(1-(4-(3-(4-Fluoro)phenylpropenoyl)amino)cyclohexyl)ethyl)-6-methoxy-2,3,4,5-tetrahydro-1H-3-benzazepine;
trans-6-Methoxy-3-(2-(1-(4-(4-quinolinyl)carboxamido)cyclohexyl)ethyl)-2,3,4,5-tetrahydro-1H-3-benzazepine;
trans-6-Methoxy-3-(2-(1-(4-(3-pyrrolo[2,3-b]pyridyl)carboxamido)cyclohexyl)ethyl)-2,3,4,5-tetrahydro-1H-3-benzazepine;
trans-(E)-7-Cyano-3-(2-(1-(4-(3-(4-fluoro)phenylpropenoyl)amino)cyclohexyl)ethyl)-2,3,4,5-tetrahydro-1H-3-benzazepine;
trans-7-Cyano-3-(2-(1-(4-(3-(3-(5-methyl)-1,2,4-oxadiazolyl)benzoyl)amino)cyclohexyl)ethyl)-2,3,4,5-tetrahydro-1H-3-benzazepine;
trans-(E)-7-Cyano-3-(2-(1-(4-(3-(3-acetylamino)phenylpropenoyl)amino)cyclohexyl)ethyl)-2,3,4,5-tetrahydro-1H-3-benzazepine;
trans-7-Cyano-3-(2-(1-(4-(6-(3,4-dihydro-3-oxo)-2H-benzoxazinyl)carboxamido)cyclohexyl)ethyl)-2,3,4,5-tetrahydro-1H-benzazepine;
trans-(E)-7-Cyano-3-(2-(1-(4-(3-(6-(1,2-dihydro-2-oxo)quinolinyl)propenoyl)amino)cyclohexyl)ethyl)-2,3,4,5-tetrahydro-1H-3-benzazepine;
trans-(E)-7-Cyano-3-(2-(1-(4-(3-(2-fluoro-4-acetylamino)phenylpropenoyl)amino)cyclohexyl)ethyl)-2,3,4,5-tetrahydro-1H-3-benzazepine;
trans-(E)-7-Cyano-3-(2-(1-(4-(3-(8-(1,2-dihydro-2-oxo)quinolinyl)propenoyl)amino)cyclohexyl)ethyl)-2,3,4,5-tetrahydro-1H-3-benzazepine;
trans-3-(2-(1-(4-(3-(3-(5-Methyl)-1,2,4-oxadiazolyl)benzoyl)amino)cyclohexyl)ethyl)-7-methylsulphonyl-2,3,4,5-tetrahydro-1H-3-benzazepine;
trans-7-(5-(3-Methyl)-1,2,4-oxadiazolyl)-3-(2-(1-(4-(3-pyrrolo[2,3-b]pyridyl)carboxamido)cyclohexyl)ethyl)-2,3,4,5-tetrahydro-1H-3-benzazepine;
trans-(E)-7-(3-(5-Methyl)-1,2,4-oxadiazolyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(5-(3-Methyl)isoxazolyl)-3-(2-(1-(4-(3-(4-fluoro)phenylpropenoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(4-fluoro)phenylacetamido)cyclohexyl)ethyl)-2,3,4,5-tetrahydro-1H-3-benzazepine;
trans-7-Cyano-3-(2-(1-(4-(2,5-difluoro)phenylacetamido)cyclohexyl)ethyl)-2,3,4,5-tetrahydro-1H-3-benzazepine;
trans-(E)-7-Cyano-3-(2-(1-(4-(3-(2,4-difluoro)phenylpropenoyl)amino)cyclohexyl)ethyl)-2,3,4,5-tetrahydro-1H-3-benzazepine;
trans-(E)-7-Cyano-3-(2-(1-(4-(3-(2,5-difluoro)phenylpropenoyl)amino)cyclohexyl)ethyl)-2,3,4,5-tetrahydro-1H-3-benzazepine;
trans-7-Cyano-3-(2-(1-(4-(3-phenylpropanoyl)amino)cyclohexyl)ethyl)-2,3,4,5-tetrahydro-1H-3-benzazepine;
trans-(E)-7-Cyano-3-(2-(1-(4-(3-(4-methoxy)phenylpropenoyl)amino)cyclohexyl)ethyl)-2,3,4,5-tetrahydro-1H-3-benzazepine;
trans-(E)-7-Cyano-3-(2-(1-(4-(3-(2-acetyl)phenylpropenoyl)amino)cyclohexyl)ethyl)-2,3,4,5-tetrahydro-1H-3-benzazepine;
trans-(E)-7-Cyano-3-(2-(1-(4-(3-(4-acetyl)phenylpropenoyl)amino)cyclohexyl)ethyl)-2,3,4,5-tetrahydro-1H-3-benzazepine;
trans-(E)-7-Cyano-3-(2-(1-(4-(3-(2-cyano)phenylpropenoyl)amino)cyclohexyl)ethyl)-2,3,4,5-tetrahydro-1H-3-benzazepine;
trans-(E)-7-Cyano-3-(2-(1-(4-(3-(3-cyano)phenylpropenoyl)amino)cyclohexyl)ethyl)-2,3,4,5-tetrahydro-1H-3-benzazepine;
trans-7-Cyano-3-(2-(1-(4-(3-(5-(3-methyl)isoxazolyl)benzoyl)amino)cyclohexyl)ethyl)-2,3,4,5-tetrahydro-1H-3-benzazepine;
trans-(2)-7-Cyano-3-(2-(1-(4-(3-phenylpropenoyl)amino)cyclohexyl)ethyl)-2,3,4,5-tetrahydro-1H-3-benzazepine;
trans-(E)-7-Cyano-3-(2-(1-(4-(3-(2-pyridyl)propenoyl)amino)cyclohexyl)ethyl)-2,3,4,5-tetrahydro-1H-3-benzazepine;
trans-(E)-7-Cyano-3-(2-(1-(4-(3-(1-(4-fluoro)naphthyl)propenoyl)amino)cyclohexyl)ethyl)-2,3,4,5-tetrahydro-1H-3-benzazepine;
trans-(E)-7-Cyano-3-(2-(1-(4-(3-(6-benzodioxanyl)propenoyl)amino)cyclohexyl)ethyl)-2,3,4,5-tetrahydro-1H-3-benzazepine;
trans-(E)-7-Cyano-3-(2-(1-(4-(3-(3-(5-fluoro)indolyl)propenoyl)amino)cyclohexyl)ethyl)-2,3,4,5-tetrahydro-1H-3-benzazepine;
trans-(E)-7-Cyano-3-(2-(1-(4-(3-(3-(1-methyl)benzimidazolyl)propenoyl)amino)cyclohexyl)ethyl)-2,3,4,5-tetrahydro-1H-3-benzazepine;
trans-(E)-7-Cyano-3-(2-(1-(4-(3-(7-benzofuranyl)propenoyl)amino)cyclohexyl)ethyl)-2,3,4,5-tetrahydro-1H-3-benzazepine;
trans-(E)-7-Cyano-3-(2-(1-(4-(3-(5-(3-methyl)indolyl)propenoyl)amino)cyclohexyl)ethyl)-2,3,4,5-tetrahydro-1H-3-benzazepine;
trans-(E)-7-Cyano-3-(2-(1-(4-(3-(6-(2,3-dihydro-2-oxo)indolyl)propenoyl)amino)cyclohexyl)ethyl)-2,3,4,5-tetrahydro-1H-3-benzazepine;
trans-7-Cyano-3-(2-(1-(2-benzofuranylacetamido)cyclohexyl)ethyl)-2,3,4,5-tetrahydro-1H-3-benzazepine;
trans-(E)-7-Cyano-3-(2-(1-(4-(3-(4-(2-methyl)indolyl)propenoyl)amino)cyclohexyl)ethyl)-2,3,4,5-tetrahydro-1H-3-benzazepine;
trans-(E)-7-Cyano-3-(2-(1-(4-(3-(5-benzimidazolyl)propenoyl)amino)cyclohexyl)ethyl)-2,3,4,5-tetrahydro-1H-3-benzazepine;
trans-(E)-7-Cyano-3-(2-(1-(4-(3-phenylpropenoyl)amino)cyclohexyl)ethyl)-2,3,4,5-tetrahydro-1H-3-benzazepine;
trans-(E)-7-Cyano-3-(2-(1-(4-(3-(2,3-methylenedioxy)phenylpropenoyl)amino)cyclohexyl)ethyl)-2,3,4,5-tetrahydro-1H-3-benzazepine;
trans-(E)-7-Cyano-3-(2-( 1-(4-(3-(3-(1-(2-oxo)pyrrolidinyl))phenylpropenoyl)amino)cyclohexyl)ethyl)-2,3,4,5-tetrahydro-1H-3-benzazepine;
trans-7-Cyano-3-(2-(1-(4-(2-indolylacetamido)cyclohexyl)ethyl)-2,3,4,5-tetrahydro-1H-3-benzazepine;
trans-7-Cyano-3-(2-(1-(4-(2-benzothiophenylacetamido)cyclohexyl)ethyl)-2,3,4,5-tetrahydro-1H-3-benzazepine;
trans-(E)-7-Cyano-3-(2-(1-(4-(3-(2-(3-bromo)thiophenyl)propenoyl)amino)cyclohexyl)ethyl)-2,3,4,5-tetrahydro-1H-3-benzazepine;
trans-7-Cyano-3-(2-(1-(4-(3-(2-pyridyl)benzoyl)amino)cyclohexyl)ethyl)-2,3,4,5-tetrahydro-1H-3-benzazepine;
trans-7-Cyano-3-(2-(1-(4-(3-(5-pyrimidinyl)benzoyl)amino)cyclohexyl)ethyl)-2,3,4,5-tetrahydro-1H-3-benzazepine;
trans-7-Cyano-3-(2-(1-(4-(3-(4-cyanophenyl)benzoyl)amino)cyclohexyl)ethyl)-2,3,4,5-tetrahydro-1H-3-benzazepine;
trans-(E)-7-Cyano-3-(2-(1-(4-(3-(2-thiophenyl)propenoyl)amino)cyclohexyl)ethyl)-2,3,4,5-tetrahydro-1H-3-benzazepine;
trans-(E)-7-Cyano-3-(2-(1-(4-(3-(2-furanyl)propenoyl)amino)cyclohexyl)ethyl)-2,3,4,5-tetrahydro-1H-3-benzazepine;
trans-(E)-7-Cyano-3-(2-(1-(4-(3-(3-thiophenyl)propenoyl)amino)cyclohexyl)ethyl)-2,3,4,5-tetrahydro-1H-3-benzazepine;
trans-(E)-7-Cyano-3-(2-(1-(4-(3-(3-furanyl)propenoyl)amino)cyclohexyl)ethyl)-2,3,4,5-tetrahydro-1H-3-benzazepine;
trans-(E)-7-Cyano-3-(2-(1-(4-(3-(4-quinolinyl)propenoyl)amino)cyclohexyl)ethyl)-2,3,4,5-tetrahydro-1H-3-benzazepine;
trans-(E)-7-Cyano-3-(2-(1-(4-(3-(5-pyrimidinyl)propenoyl)amino)cyclohexyl)ethyl)-2,3,4,5-tetrahydro-1H-3-benzazepine;
trans-7-Cyano-3-(2-(1-(4-(2,4-difluoro)phenylacetamido)cyclohexyl)ethyl)-2,3,4,5-tetrahydro-1H-3-benzazepine;
trans-7-Cyano-3-(2-(1-(4-(6-(2-amino)benzothiazolyl)acetamido)cyclohexyl)ethyl)-2,3,4,5-tetrahydro-1H-3-benzazepine;
trans-7-Cyano-3-(2-(1-(4-(6-(2-methyl)benzothiazolyl)acetamido)cyclohexyl)ethyl)-2,3,4,5-tetrahydro-1H-3-benzazepine;
trans-(E)-7-Cyano-3-(2-(1-(4-(3-(4-methylaminocarbonyl)phenylpropenoyl)amino)cyclohexyl)ethyl)-2,3,4,5-tetrahydro-1H-3-benzazepine;
trans-7-Cyano-3-(2-(1-(4-(5-(2-amino)benzoxazolyl)acetamido)cyclohexyl)ethyl)-2,3,4,5-tetrahydro-1H-3-benzazepine;
trans-7-Cyano-3-(2-(1-(4-(3-(1-pyrazolyl)benzoyl)amino)cyclohexyl)ethyl)-2,3,4,5-tetrahydro-1H-3-benzazepine;
trans-7-Cyano-3-(2-(1-(4-(2-thiophenyl)acetamido)cyclohexyl)ethyl)-2,3,4,5-tetrahydro-1H-3-benzazepine;
trans-7-Cyano-3-(2-(1-(4-(3-benzothiophenyl)acetamido)cyclohexyl)ethyl)-2,3,4,5-tetrahydro-1H-3-benzazepine;
trans-7-Cyano-3-(2-(1-(4-(3-(2-(5-methyl)-1,3,4-oxadiazolyl)benzoyl)amino)cyclohexyl)ethyl)-2,3,4,5-tetrahydro-1H-3-benzazepine;
trans-(E)-7-Cyano-3-(2-(1-(4-(3-(2-naphthyl)propenoyl)amino)cyclohexyl)ethyl)-2,3,4,5-tetrahydro-1H-3-benzazepine;
trans-(E)-7-Cyano-3-(2-(1-(4-(3-(5-(3-acetyl)indolyl)propenoyl)amino)cyclohexyl)ethyl)-2,3,4,5-tetrahydro-1H-3-benzazepine;
trans-7-Cyano-3-(2-(1-(4-(3-(5-(3-methyl)-1,2,4-oxadiazolyl)benzoyl)amino)cyclohexyl)ethyl)-2,3,4,5-tetrahydro-1H-3-benzazepine;
trans-7-Cyano-3-(2-(1-(4-(5-(2-methyl)benzimidazolyl)acetamido)cyclohexyl)ethyl)-2,3,4,5-tetrahydro-1H-3-benzazepine;
trans-(E)-7-Cyano-3-(2-(1-(4-(3-(3-(2-acetyl)furanyl)propenoyl)amino)cyclohexyl)ethyl)-2,3,4,5-tetrahydro-1H-3-benzazepine;
trans-7-Cyano-3-(2-(1-(4-(6-(2-amino)benzoxazolyl)acetamido)cyclohexyl)ethyl)-2,3,4,5-tetrahydro-1H-3-benzazepine;
trans-7-(5-(3-Methyl)-1,2,4-oxadiazolyl)-3-(2-(1-(4-(2-benzothiophenyl)acetamido)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(3-thienyl)propenoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(5-quinolinyl)carboxamido)cyclohexyl)ethyl)-2,3,4,5-tetrahydro-1H-3-benzazepine;
trans-7-(5-(3-Methyl)-1,2,4-oxadiazolyl)-3-(2-(1-(4-(3-(3-(5-Methyl)-1,2,4-oxadiazolyl)benzoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(8-(1,4-dihydro-4-oxo)quinolinyl)carboxamido)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(3-fluoro)phenylpropenoyl)amino)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(3-acetamido-2-fluoro)phenylpropenoyl)amino)cyclohexyl)ethyl)-2,3,4,5-tetrahydro-1H-3-benzazepine;
trans-(E)-7-(5-(3-Methyl)-1,2,4-oxadiazolyl)-3-(2-(1-(4-(3-(3-acetyl)phenylpropenoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(3-fluoro)phenylacetamido)cyclohexyl)ethyl)-2,3,4,5-tetrahydro-1H-3-benzazepine;
trans-7-(5-(3-Methyl)-1,2,4-oxadiazolyl)-3-(2-(1-(4-(2,4-difluoro)phenylacetamido)cyclohexyl)ethyl)-2,3,4,5-tetrahydro-1H-3-benzazepine;
trans-7-(5-(3-Methyl)-1,2,4-oxadiazolyl)-3-(2-(1-(4-(2-naphthyl)acetamido)cyclohexyl)ethyl)-2,3,4,5-tetrahydro-1H-3-benzazepine;
trans-7-(5-(3-Methyl)-1,2,4-oxadiazolyl)-3-(2-(1-(4-(7-(3,4-dihydro-3-oxo)-2H-benzoxazinyl)carboxamido)cyclohexyl)ethyl)-2,3,4,5-tetrahydro-1H-3-benzazepine;
trans-7-(5-(3-Methyl)-1,2,4-oxadiazolyl)-3-(2-(1-(4-(2-fluoro)phenylacetamido)cyclohexyl)ethyl)-2,3,4,5-tetrahydro-1H-3-benzazepine;
trans-3-(2-(1-(4-(5-(2-Methyl)quinolinyl)carboxamide)cyclohexyl)ethyl)-7-methanesulphonyloxy-2,3,4,5-tetrahydro-1H-3-benzazepine;
trans-7-(5-(3-Methyl)-1,2,4-oxadiazolyl)-3-(2-(1-(4-(2-indolyl)carboxamido)cyclohexyl)ethyl)-2,3,4,5-tetrahydro-1H-3-benzazepine;
trans-7-(3-(5-Methyl)-1,2,4-oxadiazolyl)-3-(2-(1-(4-(2-benzothiophenyl)acetamido)cyclohexyl)ethyl)-2,3,4,5-tetrahydro-1H-3-benzazepine;
trans-(E)-7-(3-(5-Methyl)-1,2,4-oxadiazolyl)-3-(2-(1-(4-(3-(3-thienyl)propenoyl)amino)cyclohexyl)ethyl)-2,3,4,5-tetrahydro-1H-3-benzazepine;
trans-7-(3-(5-Methyl)-1,2,4-oxadiazolyl)-3-(2-(1-(4-(5-quinolinyl)carboxamido)cyclohexyl)ethyl)-2,3,4,5-tetrahydro-1H-3-benzazepine;
trans-7-(3-(5-Methyl)-1,2,4-oxadiazolyl)-3-(2-(1-(4-(3-pyrrolo[2,3-b]pyridyl)carboxamido)cyclohexyl)ethyl)-2,3,4,5-tetrahydro-1H-3-benzazepine;
trans-7-(3-(5-Methyl)-1,2,4-oxadiazolyl)-3-(2-(1-(4-(8-(1,4-dihydro-4-carboxamido)cyclohexyl)ethyl)-2,3,4,5-tetrahydro-1H-3-benzazepine;
trans-7-(3-(5-Methyl)-1,2,4-oxadiazolyl)-3-(2-(1-(4-(3-(3-(5-Methyl)-1,2,4-oxadiazolyl)benzoyl)amino)cyclohexyl)ethyl)-2,3,4,5-tetrahydro-1H-3-benzazepine;
trans-(E)-7-(3-(5-Methyl)-1,2,4-oxadiazolyl)-3-(2-( 1-(4-(3-(3-fluoro)phenylpropenoyl)amino)cyclohexyl)ethyl)-2,3,4,5-tetrahydro-1H-3-benzazepine;
trans-(E)-7-(3-(5-Methyl)-1,2,4-oxadiazolyl)-3-(2-(1-(4-(3-(2-fluoro)phenylpropenoyl)amino)cyclohexyl)ethyl)-2,3,4,5-tetrahydro-1H-3-benzazepine;
trans-7-(5-(3-Methyl)isoxazolyl)-3-(2-(1-(4-(2-fluoro)phenylacetamido)cyclohexyl)ethyl)-2,3,4,5-tetrahydro-1H-3-benzazepine;
trans-7-(3-(5-Methyl)isoxazolyl)-3-(2-(1-(4-(4-fluoro)phenylacetamido)cyclohexyl)ethyl)-2,3,4,5-tetrahydro-1H-3-benzazepine;
trans-(E)-7-(2-(5-Methyl)oxazolyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(3-(5-Methyl)isoxazolyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(5-Pyrimidyl)-3-(2-(1-(4-(3-(4-fluoro)phenylpropenoyl)amino)cyclohexyl)ethyl)-2,3,4,5-tetrahydro-1H-3-benzazepine;
trans-7-(5-Pyrimidinyl)-3-(2-(1-(4-(3-(3-(5-Methyl)-1,2,4-oxadiazolyl)benzoyl)amino)cyclohexyl)ethyl)-2,3,4,5-tetrahydro-1H-3-benzazepine;
trans-7-(3-(5-Methyl)isoxazolyl)-3-(2-(1-(4-(5-(2-methyl)quinolinyl)carboxamido)cyclohexyl)ethyl)-2,3,4,5-tetrahydro-1H-3-benzazepine; and
trans-3-(2-(1-(4-(3-(2-(5-Methyl)oxazolyl)benzoyl)amino)cyclohexyl)ethyl)-7-methylsulphonyloxy-2,3,4,5-tetrahydro-1H-3-benzazepine.

3. A compound according to claim 1 consisting of the group selected from:
trans-7-Cyano-3-(2-(1-(4-(3-pyrrolo[2,3-b]pyridyl)carboxamido)cyclohexyl)ethyl)-2,3,4,5-tetrahydro-1H-3-benzazepine;
trans-7-Cyano-3-(2-(1-(2-naphthylacetamido)cyclohexyl)ethyl)-2,3,4,5-tetrahydro-1H-3-benzazepine;
trans-(E)-7-Cyano-3-(2-(1-(4-(3-(2-fluoro)phenylpropenoyl)amino)cyclohexyl)ethyl)-2,3,4,5-tetrahydro-1H-3-benzazepine;
trans-(E)-7-Cyano-3-(2-(1-(4-(3-(3-fluoro)phenylpropenoyl)amino)cyclohexyl)ethyl)-2,3,4,5-tetrahydro-1H-3-benzazepine;
trans-7-Cyano-3-(2-(1-(4-(8-(1,4-dihydro-4-oxo)quinolinyl)carboxamido)cyclohexyl)ethyl)-2,3,4,5-tetrahydro-1H-3-benzazepine;
trans-7-Cyano-3-(2-(1-(4-(2-naphthyl)carboxamido)cyclohexyl)ethyl)-2,3,4,5-tetrahydro-1H-3-benzazepine;
trans-7-Cyano-3-(2-(1-(4-(2-naphthyl)carboxamido)cyclohexyl)ethyl)-2,3,4,5-tetrahydro-1H-3-benzazepine;
trans-(E)-7-Cyano-3-(2-(1-(4-(3-(2-methoxy)phenylpropenoyl)amino)cyclohexyl)ethyl)-2,3,4,5-tetrahydro-1H-3-benzazepine;
trans-(E)-7-Cyano-3-(2-(1-(4-(3-(3-methoxy)phenylpropenoyl)amino)cyclohexyl)ethyl)-2,3,4,5-tetrahydro-1H-3-benzazepine;
trans-7-Cyano-3-(2-(1-(4-(7-(1,2-dihydro-2-oxo)quinolinyl)carboxamido)cyclohexyl)ethyl)-2,3,4,5-tetrahydro-1H-3-benzazepine;
trans-7-Cyano-3-(2-(1-(4-(3-(3-(5-ethyl)-1,2,4-oxadiazolyl)benzoyl)amino)cyclohexyl)ethyl)-2,3,4,5-tetrahydro-1H-3-benzazepine;
trans-7-Cyano-3-(2-(1-(4-(1-naphthyl)acetamido)cyclohexyl)ethyl)-2,3,4,5-tetrahydro-1H-3-benzazepine;
trans-7-Cyano-3-(2-(1-(4-(6-(2,3-dihydro-2-oxo)indolyl)acetamido)cyclohexyl)ethyl)-2,3,4,5-tetrahydro-1H-3-benzazepine;
trans-7-Cyano-3-(2-(1-(4-(5-(2,3-dihydro-2-oxo)indolyl)acetamido)cyclohexyl)ethyl)-2,3,4,5-tetrahydro-1H-3-benzazepine;
trans-7-Cyano-3-(2-(1-(4-(6-(1,2-dihydro-2-oxo)quinolinyl)acetamido)cyclohexyl)ethyl)-2,3,4,5-tetrahydro-1H-3-benzazepine;
trans-7-Cyano-3-(2-(1-(4-(7-(1,2-dihydro-2-oxo)quinolinyl)acetamido)cyclohexyl)ethyl)-2,3,4,5-tetrahydro-1H-3-benzazepine;
trans-7-Cyano-3-(2-(1-(4-(6-quinoxalinyl)acetamido)cyclohexyl)ethyl)-2,3,4,5-tetrahydro-1H-3-benzazepine;
trans-7-Cyano-3-(2-(1-(4-(6-(3,4-dihydro-2-oxo)-2H-benzoxazinyl)acetamido)cyclohexyl)ethyl)-2,3,4,5-tetrahydro-1H-3-benzazepine;
trans-(E)-7-Cyano-3-(2-(1-(4-(3-(2-fluoro-5-acetamido)phenylpropenoyl)amino)cyclohexyl)ethyl)-2,3,4,5-tetrahydro-1H-3-benzazepine;
trans-7-(5-(3-Methyl)-1,2,4-oxadiazolyl)-3-(2-(1-(4-(5-(2-methyl)quinolinyl)carboxamido)cyclohexyl)ethyl)-2,3,4,5-tetrahydro-1H-3-benzazepine;
trans-3-(2-(1-(4-(3-(2-(4-Methyl)oxazolyl)benzoyl)amino)cyclohexyl)ethyl)-7-methylsulphonyl-2,3,4,5-tetrahydro-1H-3-benzazepine;
trans-3-(2-(1-(4-(3-trifluoromethylbenzoyl)amino)cyclohexyl)ethyl)-7-methylsulphonyl-2,3,4,5-tetrahydro-1H-3-benzazepine;
trans-7-(5-Pyrimidinyl)-3-(2-(1-(4-(5-(2-methyl)quinolinyl)carboxamido)cyclohexyl)ethyl)-2,3,4,5-tetrahydro-1H-3-benzazepine;
trans-(E)-7-(2-Pyridyl)-3-(2-(1-(4-(3-(2-cyano)phenylpropenoyl)amino)cyclohexyl)ethyl)-2,3,4,5-tetrahydro-1H-3-benzazepine;
trans-(E)-7-(2-Pyridyl)-3-(2-(1-(4-(3-(3-cyano)phenylpropenoyl)amino)cyclohexyl)ethyl)-2,3,4,5-tetrahydro-1H-3-benzazepine; and
trans-(E)-7-(2-Pyridyl)-3-(2-(1-(4-(3-(4-cyano)phenylpropenoyl)amino)cyclohexyl)ethyl)-2,3,4,5-tetrahydro-1H-3-benzazepine.

4. A compound according to claim 1 selected from the group consisting of:
trans-(E)-7-Cyano-3-(2-(1-(4-(5-quinolinyl)carboxamido)cyclohexyl)ethyl)-2,3,4,5-tetrahydro-1H-benzazepine;
trans-7-Cyano-3-(2-(1-(4-(5-(8-fluoro)quinolinyl)carboxamido)cyclohexyl)ethyl)-2,3,4,5-tetrahydro-1H-3-benzazepine;
trans-3-(2-(1-(4-(5-(2-Methyl)quinolinyl)carboxamido)cyclohexyl)ethyl)-7-methylsulphonyl-2,3,4,5-tetrahydro-1H-3-benzazepine;
trans-3-(2-(1-(4-(5-(2-Methyl)quinolinyl)carboxamide)cyclohexyl)ethyl)-7-methanesulphonyloxy-2,3,4,5-tetrahydro-1H-3-benzazepine;
trans-(E)-3-(2-(1-(4-(3-(4-fluoro)phenylpropenoyl)amino)cyclohexyl)ethyl)-7-methylsulphonyl-2,3,4,5-tetrahydro-1H-3-benzazepine;
trans-3-(2-(1-(4-(5-(8-Chloro-2-methyl)quinolinyl)carboxamide)cyclohexyl)ethyl)-7-methanesulphonyl-2,3,4,5-tetrahydro-1H-3-benzazepine;
trans-(E)-7-(2-Pyridyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(2-Pyrimidyl)-3-(2-(1-(4-(3-(4-fluoro)phenylpropenoyl)amino)cyclohexyl)ethyl)-2,3,4,5-tetrahydro-1H-3-benzazepine;
trans-(E)-7-(1-Pyrrolidinylcarbonyl)-3-(2-(1-(4-(3-(4-fluoro)phenylpropenoyl)amino)cyclohexyl)ethyl)-2,3,4,5-tetrahydro-1H-3-benzazepine;
trans-7-(1-Pyrrolidinylcarbonyl)-3-(2-(1-(4-(3-pyrrolo[2,3-b]pyridyl)carboxamido)cyclohexyl)ethyl)-2,3,4,5-tetrahydro-1H-3-benzazepine;
trans-3-(2-(1-(4-(5-(8-Fluoro-2-methyl)quinolinyl)carboxamido)cyclohexyl)ethyl)-7-methylsulphonyl-2,3,4,5-tetrahydro-1H-3-benzazepine;
trans-3-(2-(1-(4-(5-(8-Fluoro-2-methyl)quinolinyl)carboxamido)cyclohexyl)ethyl)-7-methylsulphonyloxy-2,3,4,5-tetrahydro-1H-3-benzazepine.

5. A pharmaceutical composition for the treatment of muscarinic acetylcholine receptor mediated diseases comprising a compound according to claim 1 and a pharmaceutically acceptable carrier thereof.

6. A method of inhibiting the binding of acetylcholine to its receptors in a mammal in need thereof comprising administering a safe and effective amount of a compound according to claim 1.

7. A method of treating a muscarinic acetylcholine receptor mediated disease, wherein acetylcholine binds to said receptor, comprising administering a safe and effective amount of a compound according to claim 1.

8. A method according to claim 7 wherein the disease is selected from the group consisting of chronic obstructive lung disease, chronic bronchitis, asthma, chronic respiratory obstruction, pulmonary fibrosis, pulmonary emphysema and allergic rhinitis.

9. A method according to claim 8 wherein administration is via inhalation via the mouth or nose.

10. A method according to claim 9 wherein administration is via a medicament dispenser selected from a reservoir dry powder inhaler, a multi-dose dry powder inhaler or a metered dose inhaler.

11. A method according to claim 10 wherein the compound is administered to a human and has a duration of action of 12 hours or more for a 1 mg dose.

12. A method according to claim 11 wherein the compound has a duration of action of 24 hours or more.

13. A method according to claim 12 wherein the compound has a duration of action of 36 hours or more.
